(19) United States
(12) Patent Application Publication
(10) Pub. No.: US 2026/0107929 A1
(43) Pub. Date: Apr. 23, 2026

(54) NON-HUMAN ANIMALS COMPRISING A MODIFIED TRANSFERRIN RECEPTOR LOCUS

(71) Applicant: Regeneron Pharmaceuticals, Inc., Tarrytown, NY (US)

(72) Inventors: Andrew J. Murphy, Croton-on-Hudson, NY (US); Yajun Tang, White Plains, NY (US); Qing Fang, Chappaqua, NY (US); Maria Praggastis, Cortlandt Manor, NY (US); Jun Zhong, Fair Lawn, NJ (US); Katherine Cygnar, New York, NY (US); Alexander O. Mujica, Elmsford, NY (US)

(21) Appl. No.: 19/099,378

(22) PCT Filed: Jul. 28, 2023
(86) PCT No.: PCT/US2023/071261

§ 371 Date: Jan. 28, 2025

Related U.S. Application Data
(60) Provisional application No. 63/369,842, filed on Jul. 29, 2022.

(51) Int. Cl. A01K 67/0276; A01K 67/0271; C07K 14/47; C12N 5/071; C12N 15/10; C12N 15/11

(52) U.S. Cl. CPC A01K 67/0276; A01K 67/0271; C07K 14/47; C12N 5/0602; C12N 15/10; C12N 15/11; A01K 2207/12; A01K 2207/15; A01K 2217/075; A01K 2227/105; A01K 2267/0306; C12N 2510/00

(57) ABSTRACT

Non-human animal cells and non-human animals comprising a humanized Tfrc gene, e.g., at an endogenous Tfrc locus, and methods of using such non-human animal cells and non-human animals are provided. Non-human animal cells or non-human animals comprising a humanized Tfrc gene express a human TfR protein or fragments thereof. Non-human animal cells and non-human animals comprising a humanized Tfrc gene and a knockout mutation in a Gaa gene, e.g., at an endogenous Gaa locus, and methods of using such non-human animal cells and non-human animals are also provided. Such animals are useful to screen anti-human-TfR binding protein based therapies of, e.g., Pompe disease.

### FIELD OF THE INVENTION

A genetically modified non-human animal (e.g., a rodent, e.g., a mouse, or a rat) comprising in its genome a nucleic acid encoding a human (h) Transferrin Receptor (TfR) protein, or a portion thereof, is described. Thus, genetically modified non-human animals that express hTfR protein, or a portion thereof, e.g., on the surface of a cell, e.g., an endothelial blood brain barrier (BBB) cell, are also described. Such genetically modified non-human animals that express human TfR protein, or a portion thereof, on the surface of a cell e.g., a BBB endothelial cell, may be used as models for preclinical testing of therapeutics, e.g., TfR-based binding proteins, which may be useful for mediating the intracellularization and/or transport of therapeutic molecules across the blood brain barrier via TfR.

### SEQUENCE LISTING

A Sequence Listing in xml format entitled “11297WO01_xml.xml,” which was created Jul. 21, 2023, and is 140 Kb, is incorporated herein by reference in its entirety.

### BACKGROUND

Iron delivery to the brain is accomplished via binding and intracellular trafficking of the iron binding protein transferrin (Tf). The Tf receptor (TfR) is a target of some studies to deliver therapeutics intracellular and/or to the brain; however, many of these drug delivery approaches have shortcomings. Targeting efficiencies have also been compromised depending on the trafficking mechanisms at the BBB and whether a CNS disease state has altered the integrity of the barrier.

A myriad of medical conditions could be benefit from the successful intracellularization of therapeutics and/or transport of therapeutics across the blood brain barrier. Thus, there remains a need for animal models that may be useful in testing the efficacy of certain biologics to transport therapeutics across the blood brain barrier.

### SUMMARY

Provided herein are genetically modified non-human animals having recombinant genetic loci encoding a human Transferrin Receptor (TfR) protein. Also provided herein are compositions and methods for generating and using such modified non-human animals, including such modified non-human animals having a recombinant genetic locus encoding a human Transferrin Receptor (TfR) protein and a knockout mutation in the α-glucosidase (GAA) locus, which animals may be useful for testing delivery of a therapeutic GAA across the blood brain barrier using TfR.

Described herein are genetically engineered non-human animal (e.g., mammalian, rodent, rat, mouse) genomes, engineered non-human animal (e.g., mammalian, rodent, rat, mouse) cells, and non-human animals (e.g., mammalian, rodent, rat, mouse) comprising a heterologous (e.g., human) TFRC gene, or a portion thereof. In some embodiments, genetically engineered animals described herein express a heterologous (e.g., human) TfR protein from a desired locus (e.g., from an endogenous Tfrc segment). The non-human animal may be a mammal, such as a rodent (e.g., a mouse or a rat). The non-human animal cell can be a mammalian cell, such as a rodent cell (e.g., a mouse cell or a rat cell). The non-human animal genome can be a mammalian nucleic acid, such as a rodent nucleic acid (e.g., a mouse nucleic acid or a rat nucleic acid).

In some embodiments, a non-human animal, a non-human animal cell, or non-human animal genome comprises a nucleic acid sequence encoding a heterologous (e.g., human) TfR protein or portion thereof.

Also described herein is a method of making a non-human animal cell and/or a non-human animal comprising a heterologous TfR protein or portion thereof. In some embodiments, the method comprises inserting the nucleic acid sequence encoding the heterologous TfR protein or the portion thereof into the genome of the non-human animal cell. In some embodiment, the method comprises inserting a nucleic acid sequence encoding a heterologous TfR protein or the portion thereof as described herein into the genome of the non-human animal cell, or a non-human animal. In some embodiments, the method comprises inserting the nucleic acid sequence encoding the heterologous TfR protein or the portion thereof into the genome of the non-human animal embryonic stem (ES) cell, wherein the inserting comprises inserting the nucleic acid sequence encoding the heterologous TfR protein or the portion thereof into the genome of the non-human animal ES cell to form a modified non-human animal ES cell comprising the nucleic acid sequence encoding the heterologous TfR protein or the portion thereof in its genome.

In some embodiments, the nucleic acid sequence encoding the heterologous TfR protein or the portion thereof comprises: (i) a nucleic acid sequence comprising exon 2 of a human TFRC gene or a coding portion thereof; (ii) a nucleic acid sequence comprising exon 3 of a human TFRC gene or a portion thereof; (iii) a nucleic acid sequence comprising exon 4 of a human TFRC gene or a portion thereof; (iv) a nucleic acid sequence comprising exon 5 of a human TFRC gene or a portion thereof; (v) a nucleic acid sequence comprising exon 6 of a human TFRC gene or a portion thereof; (vi) a nucleic acid sequence comprising exon 7 of a human TFRC gene or a portion thereof; (vii) a nucleic acid sequence comprising exon 8 of a human TFRC gene or a portion thereof; (viii) a nucleic acid sequence comprising exon 9 of a human TFRC gene or a portion thereof; (ix) a nucleic acid sequence comprising exon 10 of a human TFRC gene or a portion thereof; (x) a nucleic acid sequence comprising exon 11 of a human TFRC gene or a portion thereof; (xi) a nucleic acid sequence comprising exon 12 of a human TFRC gene or a portion thereof; (xii) a nucleic acid sequence comprising exon 13 of a human TFRC gene or a portion thereof; (xiii) a nucleic acid sequence comprising exon 14 of a human TFRC gene or a portion thereof; (xiv) a nucleic acid sequence comprising exon 15 of a human TFRC gene or a portion thereof; (xv) a nucleic acid sequence comprising exon 16 of a human TFRC gene or a portion thereof; (xvi) a nucleic acid sequence comprising exon 17 of a human TFRC gene or a portion thereof; (xvii) a nucleic acid sequence comprising exon 18 of a human TFRC gene or a portion thereof; (xviii) a nucleic acid sequence comprising exon 19 of a human TFRC gene or a coding portion thereof; or (xix) any combination of (i)-(xviii). In some embodiments, the nucleic acid sequence encoding the heterologous TfR protein or the portion thereof comprises: (i) a nucleic acid sequence comprising exon 2 of a human TFRC gene or a coding portion thereof and intron 2 of a human TFRC gene or a portion thereof; (ii) a nucleic acid sequence comprising exon 3 of a human TFRC gene or a portion thereof and intron 3 of a human TFRC gene or a portion thereof; (iii) a nucleic acid sequence comprising exon 4 of a human TFRC gene or a portion thereof and intron 4 of a human TFRC gene or a portion thereof; (iv) a nucleic acid sequence comprising exon 5 of a human TFRC gene or a portion thereof and intron 5 of a human TFRC gene or a portion thereof; (v) a nucleic acid sequence comprising exon 6 of a human TFRC gene or a portion thereof and intron 6 of a human TFRC gene or a portion thereof; (vi) a nucleic acid sequence comprising exon 7 of a human TFRC gene or a portion thereof and intron 7 of a human TFRC gene or a portion thereof; (vii) a nucleic acid sequence comprising exon 8 of a human TFRC gene or a portion thereof and intron 8 of a human TFRC gene or a portion thereof; (viii) a nucleic acid sequence comprising exon 9 of a human TFRC gene or a portion thereof and intron 9 of a human TFRC gene or a portion thereof; (ix) a nucleic acid sequence comprising exon 10 of a human TFRC gene or a portion thereof and intron 10 of a human TFRC gene or a portion thereof; (x) a nucleic acid sequence comprising exon 11 of a human TFRC gene or a portion thereof and intron 11 of a human TFRC gene or a portion thereof; (xi) a nucleic acid sequence comprising exon 12 of a human TFRC gene or a portion thereof and intron 12 of a human TFRC gene or a portion thereof; (xii) a nucleic acid sequence comprising exon 13 of a human TFRC gene or a portion thereof and intron 13 of a human TFRC gene or a portion thereof; (xiii) a nucleic acid sequence comprising exon 14 of a human TFRC gene or a portion thereof and intron 14 of a human TFRC gene or a portion thereof; (xiv) a nucleic acid sequence comprising exon 15 of a human TFRC gene or a portion thereof and intron 15 of a human TFRC gene or a portion thereof; (xv) a nucleic acid sequence comprising exon 16 of a human TFRC gene or a portion thereof and intron 16 of a human TFRC gene or a portion thereof; (xvi) a nucleic acid sequence comprising exon 17 of a human TFRC gene or a portion thereof and intron 17 of a human TFRC gene or a portion thereof; (xvii) a nucleic acid sequence comprising exon 18 of a human TFRC gene or a portion thereof and intron 18 of a human TFRC gene or a portion thereof; (xviii) a nucleic acid sequence comprising exon 19 of a human TFRC gene or a coding portion thereof; or (xix) any combination of (i)-(xviii). In some embodiments, the nucleic acid sequence encoding the heterologous TfR protein or the portion thereof comprises, consists essentially of, or consists of a nucleic acid sequence selected from the group consisting of a nucleic acid sequence set forth as SEQ ID NO:5, a nucleic acid sequence set forth as SEQ ID NO:6, a nucleic acid sequence set forth as SEQ ID NO:9, and a nucleic acid sequence set forth as SEQ ID NO:10. In some embodiments, the nucleic acid sequence encoding the heterologous TfR protein or the portion thereof comprises, consists essentially of, or consists of a nucleic acid sequence set forth as SEQ ID NO:9. In some embodiments, the nucleic acid sequence encoding the heterologous TfR protein or the portion thereof comprises, consists essentially of, or consists of a nucleic acid sequence set forth as SEQ ID NO:10.

In some embodiments, the nucleic acid sequence encoding the heterologous TfR protein or portion thereof is at an endogenous Tfrc locus, and may in some embodiments, replace an orthologous endogenous nucleic acid sequence encoding an endogenous TfR protein or a portion thereof.

In some embodiments, a non-human animal cell, a non-human animal, or a non-human animal genome as described herein comprises an endogenous Tfrc locus, wherein the endogenous Tfrc locus comprises a heterozygous or homozygous replacement of an endogenous nucleic acid sequence encoding an endogenous TfR protein or a portion thereof with the nucleic acid sequence encoding the heterologous TfR protein or the portion thereof, and wherein the endogenous nucleic acid sequence encoding the endogenous TfR protein or the portion thereof and the nucleic acid sequence encoding the heterologous TfR protein or the portion thereof are orthologous.

In some embodiments, a heterologous TfR protein or the portion thereof (where heterologous is in relation to a non-human animal) comprises an amino acid sequence of a human TfR protein or a portion thereof. In some embodiments, the heterologous TfR protein or the portion thereof comprises: (i) an amino acid sequence set forth as SEQ ID NO:4; (ii) an amino acid sequence set forth as SEQ ID NO:25; (iii) an amino acid sequence set forth as SEQ ID NO:26; (iv) an amino acid sequence set forth as SEQ ID NO:27; (v) an amino acid sequence set forth as SEQ ID NO:28; or (vi) any combination of (i)-(v). In some embodiments, the heterologous TfR protein or the portion thereof comprises an amino acid sequence set forth as SEQ ID NO: 25. In some embodiments, the heterologous TfR protein is a full-length human TfR protein. In some embodiments, the heterologous TfR protein (e.g., the full-length human TfR protein) is expressed on the cell surface of a non-human animal cell as described herein, e.g., a non-human animal cell isolated from a non-human animal as described herein and/or a non-human animal cell identified in Table 1. In some embodiments, the heterologous TfR protein (e.g., the full-length human TfR protein) is expressed on the cell surface of a non-human animal blood brain barrier (BBB) cell e.g., in a non-human animal as described herein. In some embodiments, the heterologous TfR protein (e.g., the full-length human TfR protein) is not expressed on the cell surface of a non-human animal cell as described herein, e.g., a non-human animal cell isolated from a non-human animal as described herein, e.g., where the non-human animal cell is a pluripotent cell, e.g., a germ cell. In some embodiments, the non-human animal cell does not express the heterologous TfR protein and may be, e.g., an embryonic stem cell, which may be an embryonic stem cell line maintained in culture.

Also described herein are chimeric nucleic acid molecules which may be useful in making a non-human animal cell, a non-human animal genome, and/or a non-human animal genome as described herein. In some embodiments, a chimeric nucleic acid molecule comprises a nucleic acid sequence of a non-human animal Tfrc gene that (a) encodes a TfR protein and (b) is modified to comprise a replacement of a sequence encoding the TfR protein or portion thereof with a homologous sequence encoding a heterologous TfR protein or a portion thereof, wherein the chimeric nucleic acid molecule encodes a functional TfR protein. In some embodiments, the chimeric nucleic acid sequence further comprises promoter and/or regulatory sequences of the non-human animal Tfrc gene. In some chimeric nucleic acid molecule embodiments, the homologous nucleic acid sequence comprises: (i) a nucleic acid sequence comprising exon 2 of a human TFRC gene or a coding portion thereof; (ii) a nucleic acid sequence comprising exon 3 of a human TFRC gene or a portion thereof; (iii) a nucleic acid sequence comprising exon 4 of a human TFRC gene or a portion thereof; (iv) a nucleic acid sequence comprising exon 5 of a human TFRC gene or a portion thereof; (v) a nucleic acid sequence comprising exon 6 of a human TFRC gene or a portion thereof; (vi) a nucleic acid sequence comprising exon 7 of a human TFRC gene or a portion thereof; (vii) a nucleic acid sequence comprising exon 8 of a human TFRC gene or a portion thereof; (viii) a nucleic acid sequence comprising exon 9 of a human TFRC gene or a portion thereof; (ix) a nucleic acid sequence comprising exon 10 of a human TFRC gene or a portion thereof; (x) a nucleic acid sequence comprising exon 11 of a human TFRC gene or a portion thereof; (xi) a nucleic acid sequence comprising exon 12 of a human TFRC gene or a portion thereof; (xii) a nucleic acid sequence comprising exon 13 of a human TFRC gene or a portion thereof; (xiii) a nucleic acid sequence comprising exon 14 of a human TFRC gene or a portion thereof; (xiv) a nucleic acid sequence comprising exon 15 of a human TFRC gene or a portion thereof; (xv) a nucleic acid sequence comprising exon 16 of a human TFRC gene or a portion thereof; (xvi) a nucleic acid sequence comprising exon 17 of a human TFRC gene or a portion thereof; (xvii) a nucleic acid sequence comprising exon 18 of a human TFRC gene or a portion thereof; (xviii) a nucleic acid sequence comprising exon 19 of a human TFRC gene or a coding portion thereof; or (xix) any combination of (i)-(xviii). In some chimeric nucleic acid embodiments, a modified non-human animal Tfrc gene further comprises a drug selection cassette. In some chimeric nucleic acid embodiments, the chimeric nucleic acid further comprises: (i) a 5′ homology arm upstream of the modified non-human animal Tfrc gene; and (ii) a 3′ homology arm downstream of the modified non-human animal Tfrc gene. In some embodiments, the 5′ homology arm and 3′ homology arm undergo homologous recombination with a non-human animal Tfrc locus of interest, and following homologous recombination with the non-human animal Tfrc locus of interest, the modified non-human animal Tfc gene replaces the non-human animal Tfrc gene at the non-human animal Tfrc locus of interest and is operably linked to an endogenous promoter that drives expression of the non-human animal Tfrc gene at the non-human animal Tfrc locus of interest. In some embodiments, a 5′ homology arm comprises a nucleic acid sequence set forth as SEQ ID NO: 7; and/or a 3′ homology arm comprises a nucleic acid sequence set forth as SEQ ID NO:8. In some embodiments, a nucleic acid sequence of a chimeric nucleic acid as described herein comprises a nucleic acid sequence set forth as SEQ ID NO:5.

Also described herein is an animal model of Pompe disease. Accordingly, described herein is a non-human animal, non-human animal cell, or non-human animal genome comprising a knockout mutation of an endogenous α-glucosidase (Gaa) gene. In some embodiments a non-human animal or non-human animal cell comprising a knockout mutation of an endogenous Gaa gene comprises an accumulation of glucose, e.g., in a lysosome, compared to a wildtype control non-human animal or non-human animal cell comprising a wildtype Gaa gene. In some embodiments, the knockout mutation comprises a deletion of the Gaa gene or a portion thereof. In some embodiments, the knockout mutation comprises a deletion of the entire coding sequence of the Gaa gene. In some embodiments, a non-human animal, a non-human animal cell, or a non-human animal genome in the Pompe disease model does not express GAA protein. In some Pompe disease model embodiments, a non-human animal, non-human animal cell, or non-human animal genome comprises an endogenous Gaa locus that comprises the sequence set forth as SEQ ID NO:50 or the sequence set forth as SEQ ID NO:51. In some Pompe disease model embodiments, a non-human animal, a non-human animal cell, or a non-human animal genome comprising a knockout mutation of an endogenous Gaa gene further comprises a nucleic acid encoding a heterologous TfR protein or a portion thereof as described herein.

Methods of knocking out an endogenous Gaa gene are also provided. In some embodiments, the methods comprise modifying an endogenous Gaa locus of the non-human animal to comprise a knockout mutation of the Gaa gene.

A non-human animal as described herein comprising a nucleic acid encoding a heterologous TfR protein or a portion thereof as described herein and/or a knockout mutation of an endogenous Gaa gene may be useful for testing an anti-human-TfR binding protein, and thus, may comprise an anti-human-TfR binding protein that binds human TfR. In some embodiments, the anti-human-TfR binding protein is fused to a therapeutic agent, e.g., α-glucosidase.

In some embodiments, the inserting of the nucleic acid comprises contacting the genome of the non-human animal, the genome of the non-human animal cell, or the non-human animal genome with any chimeric nucleic acid molecule (e.g., targeting vector) of the disclosure.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A provides identifying information for the mouse and human transferrin receptor C (TFRC) genes and transferrin receptor (TfR) proteins so encoded. FIG. 1B provides schematics (not-to scale) of the mouse and human transferrin receptor C genes and the targeting vector for the humanization of the Tfrc^{hum} mice. The asterisks indicate the locations of the (a) upstream (7228mTU) and downstream (7228mTD) primers for the loss-of-allele assay (upper panel) and (b) upstream (7228hTU) and downstream (7228hTD) primers for the gain-of-allele assay (middle panel). The floxed self-deleting hygromycin cassette (SDC hUB Hygro) is shown downstream of the human sequence, with the remainder of the mouse 3′ UTR to follow (bottom panel).

FIG. 2 provides schematics (not to scale) of the 7228 (top panel) and 7229 (bottom panel) modified alleles. Sequences of the 7228 and 7229 alleles are set forth as SEQ ID NO:9 and SEQ ID NO:10, respectively. Exon 1, intron 1, exon 2 (including the ATG start codon), part of intron 2, and the non-coding UTR of exon 19 remain mouse. The asterisks indicate the locations of the upstream (7228hTU) and downstream (7228hTD) primers for the gain-of-allele assay. The floxed self-deleting hygromycin cassette is shown downstream of the human sequence, within remainder of the mouse 3′ UTR of exon 19, in the 7228 allele. The floxed self-deleting hygromycin cassette is shown deleted within the mouse 3′ UTR of exon 19 of the 7229 allele.

An annotation of 7228 allele (SEQ ID NO:9) is as follows:

| | |
|---|---|
| Mouse Sequence | 1-5320 |
| Human Sequence | 5321-31560 |
| Start Codon | 4272-4274 |
| Exon 1 (noncoding; mouse) | 1-117 |
| Intron 1 (mouse) | 118-4248 |
| Exon 2 (coding; mouse) | 4249-4307 |
| 5′ intron 2 (mouse) | 4308-5238 |
| 3′ intron 2 (human) | 5239-5515 |
| Exon 3-stop (human) | 5516-28934 |
| Stop codon | 28932-28934 |
| Human 3′ UTR | 28935-31560 |
| Hygro Self-Deleting Cassette | 31561-36778 |
| SalI/XhoI hybrid site | 31561-31566 |
| LoxP1 | 31567-31600 |
| LoxP2 | 36708-36741 |
| I_Ceu | 36747-36772 |
| NheI | 36773-36778 |
| Mouse sequence (Tfrc 3′ UTR) | 36779-38799 |

An annotation of the 7229 allele (SEQ ID NO:10), lacking the Hygromycin cassette, is as follows:

| | |
|---|---|
| Mouse Sequence | 1-5320 |
| Human Sequence | 5321-31560 |
| Start Codon | 4272-4274 |
| Exon 1 (noncoding; mouse) | 1-117 |
| Intron 1 (mouse) | 118-4248 |
| Exon 2 (coding; mouse) | 4249-4307 |
| 5′ intron 2 (mouse) | 4308-5238 |
| 3′ intron 2 (human) | 5239-5515 |
| Exon 3-stop (human) | 5516-28934 |
| Stop codon | 28932-28934 |
| Human 3′ UTR | 28935-31560 |
| SalI/XhoI hybrid site | 31561-31566 |
| LoxP1 | 31567-31600 |
| I_Ceu | 31606-31631 |
| NheI | 31632-31637 |
| Mouse sequence (Tfrc 3′ UTR) | 31638-33658 |

FIG. 3 shows an alignment of the mouse TfR protein (mTfR; SEQ ID NO:2) with the human hTfR protein (hTfR; SEQ ID NO:4), and the TfR protein (SEQ ID NO:25) encoded by the 7228 allele (SEQ ID NO:9) or 7229 allele (SEQ ID NO:10). The cytoplasmic, transmembrane, and the extracellular domains are labeled and shown.

FIG. 4 provides identifying information for a mouse α-glucosidase (Gaa) gene and the GAA proteins so encoded, and four SpCas9 guide RNAs (gRNAs) used to collapse the Gaa allele in a mouse embryonic stem cell.

FIG. 5 provides a schematic (not to scale) of a Gaa knockout allele comprising a deletion of the Gaa gene sequence using guide RNAs that directed SpCas9 cleavage close to the Gaa start ATG (guide 9251mGU, cut site 38 bp upstream from the ATG; guide 9251mGU3, cut site 18 bp downstream of the ATG) and after the stop codon (guide 9251mGD3, cut site 677 bp downstream of the stop; guide 9251mGD4, cut site 705 bp downstream of the stop codon).

FIGS. 6A-6C shows western blots showing that anti-human TfR antibody clones deliver GAA to the cerebrum of Tfrc^{hum} mice. Each lane=1 mouse. Anti-mouse mTfR:GAA in Wt mice was used as a positive control. Anti-mouse mTfR:GAA in Tfrc^{hum} mice was used as a negative control.

FIG. 7 shows western blots showing that a subset of anti-hTfR antibody clones deliver mature GAA to the brain parenchyma in scfv:GAA format (delivery by HDD). Anti-mouse mTfR:GAA in Wt mice was used as a positive control. Anti-mouse mTfR:GAA in Tfrc^{hum} mice was used as a negative control. P=parenchyma (supernatant) fraction; E=endothelial (pellet) fraction.

FIG. 8 shows western blots showing that four selected anti-hTfR antibody clones deliver mature GAA to the brain parenchyma in scfv:GAA format (AAV8 episomal liver depot gene therapy). Anti-mouse mTfR:GAA in Wt mice was used as a positive control. Anti-mouse mTfR:GAA in Tfrc^{hum} mice was used as a negative control.

FIG. 9 shows western blots showing that three selected episomal AAV8 liver depot anti-hTfR antibody clones deliver mature GAA to the CNS, heart, and muscle in Gaa^{-/-}/Tfrc^{hum} mice.

FIG. 10 shows that three selected episomal AAV8 liver depot anti-hTfR antibody clones rescue glycogen storage in CNS, heart, and muscle in Gaa^{-/-}Tfrc^{hum} mice. Wt untreated mice were a positive control, and Gaa^{-/-} untreated mice were a negative control.

FIG. 11A-11D show that three selected episomal AAV8 liver depot anti-hTfR antibody clones rescue glycogen storage in brain thalamus (FIG. 11A), brain cerebral cortex (FIG. 11B), brain hippocampus CA1 (FIG. 11C), and quadricep (FIG. 11D) in Gaa/Tfrc^{hum} mice. Wt untreated mice were a positive control, and Gaa^{-/-} untreated mice were a negative control.

FIGS. 12A-12B show GAA expression levels in the serum, liver, cerebrum and quadricep (FIG. 12A) and glycogen levels in the cerebellum and quadricep (FIG. 12B) in Pompe disease model mice (Tfrc^{hum}/GAA^{-/-}) at 3 weeks after intravenous injection of a recombinant AAV8 anti-TfR:GAA insertion template together with LNP-gRNA. Untreated Pompe disease model mice and wild type mice were used as controls. Mice injected with a recombinant AAV8 anti-TfR:GAA episomal template were used as a positive control. Mice injected with a recombinant AAV8 anti-TfR:GAA insertion template without LNP-gRNA were used as a negative control.

### DETAILED DESCRIPTION

### I. Overview

Transferrin (Tf) and its receptors (TfRs) are central in the regulation of iron metabolism. There are two types of transferrin receptors: TfRT, also referred to as cluster of differentiation 71 (CD71), which is widely expressed and binds Tf with high affinity, and the less common TfR2, which is predominantly expressed in hepatocytes. “TfR” as used herein refers to TfR1 (CD71), unless specified otherwise.

The uptake of Tf-bound iron through TfR1 is the main source of cellular iron import. TfR1 is a 90 kDa type II transmembrane protein having 760 amino acids. TfR1 comprises a cytoplasmic N-terminal domain (amino acids 1-67), a transmembrane domain (amino acids 68-88), and a large extracellular C-terminal domain (amino acids 89-763), which C-terminal doman comprises the Tf binding site. TfR1 may be generally found as a homodimer, with the monomers linked by disulfide bonds on the cell surface. with a molecular weight of about 180 kDa.

TfR is present both in human and non-human species, such as non-human primates and rodents. An example amino acid sequence of human (h) TfR1 is set forth as SEQ ID NO:4, which is identical to the amino acid sequence of the hTfR1 protein represented as Uniprot P02786. The gene encoding for TfR, referred to as TFRC, is found on chromosome 3 in humans. TFRC comprises 19 exons. An example gene sequence for TFRC, with annotated exons and introns, can be found from the NCBI database (Gene ID: 7037). An example coding sequence for hTfR is set forth in SEQ ID NO:3.

An example amino acid sequence of mouse (m) TfR1 is set forth as SEQ ID NO:2, which is identical to the amino acid sequence of the mTfR1 protein represented as Uniprot Q62351 and which has about 77% amino acid sequence identity with hTfR1. The mouse Tfrc gene is found on chromosome 16 in mice. The complete gene sequence for mouse Tfrc, with annotated exons and introns, can be found from the NCBI database (Gene ID: 22042). An example coding sequence for mTfR is set forth in SEQ ID NO:1. Disclosed herein are non-human animal cells, non-human animals, and non-human genomes (e.g., found in non-human animal cell nuclei) comprising an exogenous TFRC sequence. In some embodiments, the exogenous sequence is incorporated in the endogenous locus of a gene.

In some embodiments, provided herein are non-human animal cells and non-human animals having a heterologous TFRC sequence in the genomes (cellular nuclei) of the non-human animal cells or non-human animals provided herein. The heterologous TFRC sequence can be inserted into an endogenous Tfrc locus, thus providing non-human animal cells and non-human animals having a genetically modified endogenous Tfrc locus.

In some embodiments, provided herein are nucleic acids encoding heterologous sequences encoding at least a portion of a TFRC sequence, and methods for making non-human animal cells and non-human animals with such nucleic acids. In some embodiments, such nucleic acids have sequences to facilitate the editing of the non-human animal (e.g., loxP sites) flanking the sequences encoding the TFRC gene.

In some embodiments, the disclosure provides methods that can be used for making such non-human animals (e.g., a rodent, e.g., a rat or a mouse), cells and/tissues derived from such non-human animals, and nucleotides (e.g., targeting vectors, genomes, etc.).

In some embodiments, the disclosure also provides a non-human animal genome comprising a genetically modified endogenous Tfrc locus having a heterologous TFRC sequence. In some embodiments, the heterologous TfR sequence encodes a TfR human protein sequence. In some embodiments, the present disclosure provides a non-human animal, a non-human animal cell, or non-human animal genome (e.g., a non-human animal cell nucleus) comprising a nucleic acid sequence encoding a heterologous TfR protein or portion thereof. Such nucleic acid sequences encoding a heterologous TfR protein or portion thereof may comprise: (i) a nucleic acid sequence comprising exon 1 of a human TFRC gene or a portion thereof; (ii) a nucleic acid sequence comprising exon 2 of a human TFRC gene or a portion thereof; (iii) a nucleic acid sequence comprising exon 3 of a human TFRC gene or a portion thereof; (iv) a nucleic acid sequence comprising exon 4 of a human TFRC gene or a portion thereof, or (v) a nucleic acid sequence comprising exon 5 of a human TFRC gene or a portion thereof; (vi) a nucleic acid sequence comprising exon 6 of a human TFRC gene or a portion thereof; (vii) a nucleic acid sequence comprising exon 7 of a human TFRC gene or a portion thereof; (viii) a nucleic acid sequence comprising exon 8 of a human TFRC gene, (ix) a nucleic acid sequence comprising exon 9 of a human TFRC gene or a portion thereof; (x) a nucleic acid sequence comprising exon 10 of a human TFRC gene or a portion thereof; (xi) a nucleic acid sequence comprising exon 11 of a human TFRC gene or a portion thereof; (xii) a nucleic acid sequence comprising exon 12 of a human TFRC gene or a portion thereof or a portion thereof, (xiii) a nucleic acid sequence comprising exon 13 of a human TFRC gene or a portion thereof; (xiv) a nucleic acid sequence comprising exon 14 of a human TFRC gene or a portion thereof; (xv) a nucleic acid sequence comprising exon 15 of a human TFRC gene or a portion thereof; (xvi) a nucleic acid sequence comprising exon 16 of a human TFRC gene or a portion thereof, (xvii) a nucleic acid sequence comprising exon 17 of a human TFRC gene or a portion thereof; (xviii) a nucleic acid sequence comprising exon 18 of a human TFRC gene or a portion thereof; (ixx) a nucleic acid sequence comprising exon 19 of a human TFRC gene or a portion thereof; (xx) or any combination of (i)-(ixx), and optionally any introns between such exons. In some embodiments, nucleic acid sequences encoding a heterologous TfR protein or portion thereof may comprise a nucleic acid sequence set forth in exon 3, 4, 5, 6, 7, 8, 9, 10, 11, 12, 13, 14, 15, 16, 17, 18, and 19 of a human TFRC gene, and optionally any introns between such exons.

In some embodiments, all or part of a TfR domain is encoded by a segment of an endogenous Tfrc locus that has been deleted and replaced with a heterologous TFRC sequence. In some embodiments, non-human animals comprising a humanized Tfrc locus and expressing a human or chimeric human/non-human TfR protein from the humanized Tfrc locus are provided, as well as methods of using such non-human animals (e.g., a rodent, e.g., a rat or a mouse), cells and/tissues derived from such non-human animals, and nucleotides (e.g., targeting vectors, genomes, etc.) useful for making such animals.

In some embodiments, described herein are non-human animals comprising a genetically modified Tfrc locus encoding a modified TfR protein, wherein the modified TfR protein comprises a domain of a human TfR sequence, and all or part of the domain is encoded by a segment of the endogenous Tfrc locus that has been deleted and replaced with an orthologous human TFRC sequence, and wherein the non-human animal expresses the modified TfR protein.

In some embodiments, a domain of the human TfR sequence is encoded by the segment of the endogenous Tfrc locus that has been deleted and replaced with a heterologous sequence. Such domains can be a human TfR extracellular domain. Suitable sequences encoding extracellular domains contemplated by the disclosure include the human extracellular domain (e.g., as set forth as SEQ ID NO:28) of the TfR protein upon translation within a cell.

In some embodiments, at least two domains of the human TfR sequence are encoded by a segment of the endogenous Tfrc locus in a humanized mouse model. Illustrative examples of non-limiting domains of the human TfR sequence include, but are not limited to, a cytoplasmic domain (see, e.g., SEQ ID NO:26), a transmembrane domain (see, e.g., SEQ ID NO:27), and an extracellular domain (see, e.g., SEQ ID NO:28). In some embodiments, all or part of each domain can be encoded by the segment of the endogenous Tfrc locus that has been deleted and replaced with an orthologous human TFRC sequence. In some embodiments, some or all of the cytoplasmic domain, the transmembrane domain, or the extracellular domain may be encoded by endogenous genome. For example, some of the cytoplasmic domain may be encoded by an endogenous Tfrc gene. In some embodiments, a part of the cytoplasmic domain may be encoded by an endogenous Tfrc gene, and the resulting cytoplasmic domain will have an amino acid sequence identical to that of the cytoplasmic domain of a human TfR protein due to degeneracy of the genetic code. In some embodiments, all or part of the cytoplasmic domain, all of the transmembrane domain, and all of the extracellular domain are encoded by the segment of the endogenous Tfrc gene that has been deleted and replaced with an orthologous human TFRC sequence. In some embodiments, all of the cytoplasmic domain, the transmembrane domain, and the extracellular domain are encoded by the segment of the endogenous Tfrc gene that has been deleted and replaced with an orthologous human TFRC sequence. Suitable sequences encoding the cytoplasmic domain(s) of the disclosure produce the human cytoplasmic domains corresponding to amino acids 1-67 (SEQ ID NO:26) of the human TfR protein upon translation within a cell. Suitable sequences encoding the transmembrane domain of the disclosure produce the human transmembrane domain corresponding to amino acids 68-88 (SEQ ID NO:27) of the human TfR protein upon translation within a cell. Consequently, in some alternative embodiments all or part of a cytoplasmic domain or the transmembrane domain is encoded by an endogenous non-human animal Tfrc gene sequence.

In some embodiments, the non-human animal or non-human animal genome (e.g., a non-human animal cell nucleus) described herein encodes an orthologous human TFRC sequence in place of an endogenous mouse Tfrc sequence. In some embodiments, the non-human animal or non-human animal genome comprises the sequence selected from the group consisting of a nucleic acid sequence set forth as SEQ ID NO:5, a nucleic acid sequence set forth as SEQ ID NO:6, a nucleic acid sequence set forth as SEQ ID NO:9, and a nucleic acid sequence set forth as SEQ ID NO:10.

In some embodiments, the human TfR amino acid sequence that is encoded by the endogenous Tfrc locus that comprises a replacement of all or part of the endogenous Tfrc sequence with a corresponding human TFRC sequence comprises a full-length amino acid sequence of human TfR, e.g., as set forth in SEQ ID NO:4 or SEQ ID NO:25.

In some embodiments the non-human animal, the non-human animal cell, or the non-human animal genome described herein is heterozygous for the genetically modified endogenous Tfrc locus. In some embodiments, the non-human animal or non-human animal genome is homozygous for the genetically modified endogenous Tfrc locus.

In some embodiments, segments of an endogenous Tfrc locus are deleted and replaced with an exogenous TFRC sequence. In some of embodiments, the endogenous Tfrc locus that has been deleted and replaced with an orthologous human TFRC sequence comprises a segment or all of exon 1, a segment or all of intron 1, a segment or all of exon 2, a segment or all of intron 2, a segment or all of exon 3, a segment or all of intron 3, a segment or all of exon 4, a segment or all of intron 4, a segment or all of exon 5, a segment or all of intron 5, a segment or all of exon 6, a segment or all of intron 6, a segment or all of exon 7, a segment or all of intron 7, a segment or all of exon 8, a segment or all of intron 8, a segment or all of exon 9, a segment or all of intron 9, a segment or all of exon 10, a segment or all of intron 10, a segment or all of exon 11, a segment or all of intron 11, a segment or all of exon 12, a segment or all of intron 12, a segment or all of exon 13, a segment or all of intron 13, a segment or all of exon 14, a segment or all of intron 14, a segment or all of exon 15, a segment or all of intron 15, a segment or all of exon 16, a segment or all of intron 16, a segment or all of exon 17, a segment or all of intron 17, a segment or all of exon 18, a segment or all of intron 18, a segment or all of exon 19, a segment of the 3′ untranslated region, or a combination of the aforementioned segments of the endogenous Tfrc locus. In some embodiments, the endogenous Tfrc locus that has been deleted and replaced with an orthologous TFRC sequence comprises some or all of intron 2, all of exon 3, all of intron 3, all of exon 4, all of intron 4, a all of exon 5, all of intron 5, all of exon 6, all of intron 6, all of exon 7, all of intron 7, all of exon 8, all of intron 8, all of exon 9, all of intron 9, all of exon 10, all of intron 10, all of exon 11, a all of intron 11, all of exon 12, all of intron 12, all of exon 13, all of intron 13, all of exon 14, all of intron 14, all of exon 15, all of intron 15, all of exon 16, all of intron 16, all of exon 17, all of intron 17, all of exon 18, all of intron 18, and some or all of exon 19 of the endogenous Tfrc locus. In some embodiments, all or part of the coding sequence of an endogenous Tfrc gene, including any intervening introns, is replaced with all or part of a coding sequence only (e.g., no introns) of a human TFRC gene such that the locus encodes a human TfR protein amino acid sequence, e.g., as set forth in SEQ ID NO:4 or SEQ ID NO:25.

In some embodiments, a human TFRC sequence may be used to replace a locus within a non-human animal or non-human cell. In some embodiments the orthologous human TFRC sequence that replaces the segment of the endogenous locus may comprise a segment or all of exon 1, a segment or all of intron 1, a segment or all of exon 2, a segment or all of intron 2, a segment or all of exon 3, a segment or all of intron 3, a segment or all of exon 4, a segment or all of intron 4, a segment or all of exon 5, a segment or all of intron 5, a segment or all of exon 6, a segment or all of intron 6, a segment or all of exon 7, a segment or all of intron 7, a segment or all of exon 8, a segment or all of intron 8, a segment or all of exon 9, a segment or all of intron 9, a segment or all of exon 10, a segment or all of intron 10, a segment or all of exon 11, a segment or all of intron 11, a segment or all of exon 12, a segment or all of intron 12, a segment or all of exon 13, a segment or all of intron 13, a segment or all of exon 14, a segment or all of intron 14, a segment or all of exon 15, a segment or all of intron 15, a segment or all of exon 16, a segment or all of intron 16, a segment or all of exon 17, a segment or all of intron 17, a segment or all of exon 18, a segment or all of intron 18, a segment or all of exon 19, a segment of the 3′ untranslated region, or a combination of the aforementioned segments of the human TFRC gene. In some embodiments the orthologous human TFRC sequence that replaces the segment of the endogenous locus may comprise a segment of or all of intron 2, all of exon 3, all of intron 3, all of exon 4, all of intron 4, a all of exon 5, all of intron 5, all of exon 6, all of intron 6, all of exon 7, all of intron 7, all of exon 8, all of intron 8, all of exon 9, all of intron 9, all of exon 10, all of intron 10, all of exon 11, a all of intron 11, all of exon 12, all of intron 12, all of exon 13, all of intron 13, all of exon 14, all of intron 14, all of exon 15, all of intron 15, all of exon 16, all of intron 16, all of exon 17, all of intron 17, all of exon 18, all of intron 18, and some or all of exon 19 of the human TFRC gene. In some embodiments the orthologous human TFRC sequence that replaces the segment of the endogenous locus may comprise the all or part of the coding sequence in exons 3-19 of the human TFRC gene.

In some embodiments, a non-human animal, a non-human animal cell, or a non-human animal genome described herein encodes a humanized coding region for the TfR protein (i.e., some mouse regulatory regions and select human non-coding/coding regions). In some embodiments, the nucleic acid sequence encoding a heterologous TfR protein or portion thereof can comprise, consists essentially of, or consist of a nucleic acid sequence encoding a human or chimeric mouse/human TfR protein, such as the nucleic acid sequence selected from the group consisting of a nucleic acid sequence set forth as SEQ ID NO:5, a nucleic acid sequence set forth as SEQ ID NO:6, a nucleic acid sequence set forth as SEQ ID NO:9, and a nucleic acid sequence set forth as SEQ ID NO:10. Any such nucleic acid can be incorporated at an endogenous Tfrc locus. In some embodiments, a nucleic acid sequence encoding the heterologous TfR protein or portion thereof can replace an orthologous endogenous nucleic acid sequence encoding an endogenous TfR protein or a portion thereof.

In some embodiments, the non-human animal is a mammal, or the non-human animal genome is a mammalian genome. In some embodiments, the non-human animal can be a rodent, or the non-human animal genome can be a rodent genome. In some embodiments, the non-human animal can be a rat or mouse, or the non-human animal genome can be a rat genome or a mouse genome.

The terms “protein,” “polypeptide,” and “peptide,” are used interchangeably herein, and include polymeric forms of amino acids of any length, including coded and non-coded amino acids and chemically or biochemically modified or derivatized amino acids. The terms also include polymers that have been modified, such as polypeptides having modified peptide backbones. The term domain can refer to any part of a protein or polypeptide having a particular function or structure.

Proteins are said to have an “N-terminus” and a “C-terminus.” The term “N-terminus” relates to the start of a protein or polypeptide, terminated by an amino acid with a free amine group (—NH₂). The term “C-terminus” relates to the end of an amino acid chain (protein or polypeptide), terminated by a free carboxyl group (—COOH).

The terms “nucleic acid” and “polynucleotide,” used interchangeably herein, include polymeric forms of nucleotides of any length, including ribonucleotides, deoxyribonucleotides, or analogs or modified versions thereof. Nucleic acids and polynucleotides can include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “genomically integrated” refers to a nucleic acid that has been introduced into a cell such that the nucleotide sequence integrates into the genome of the cell and is capable of being inherited by progeny thereof. Any protocol may be used for the stable incorporation of a nucleic acid into the genome of a cell.

The term “targeting vector” refers to a recombinant nucleic acid that can be introduced by homologous recombination, non-homologous-end-joining-mediated ligation, or any other means of recombination to a target position in the genome of a cell.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells either ex vivo or in vivo. Numerous forms of viral vectors are known.

The term “wild type” includes entities having a structure and/or activity as found in a normal (as contrasted with mutant, diseased, altered, or so forth) state or context. Wild type genes and polypeptides often exist in multiple different forms (e.g., alleles).

The expression “gross mutant phenotype” refers to a significant difference or variation in phenotype between an engineered non-human mouse of the disclosure and a “wild type.”

The term “endogenous” refers to a nucleic acid sequence that occurs naturally within a cell or non-human animal. For example, an endogenous Tfrc sequence of a non-human animal refers to a native Tfrc sequence that naturally occurs at the Tfrc locus in the non-human animal.

“Exogenous” molecules or sequences include molecules or sequences that are not normally present in a cell in that form. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two portions that do not naturally occur together in the same molecule. For example, the term “heterologous,” when used with reference to portions of a nucleic acid or portions of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by sequences not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a Cas9 protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge). A skilled artisan will understand that a nucleic acid sequence as disclosed herein encompasses variants thereof, including those variants that differ due to degeneracy of the genetic code and/or codon optimization, and that encode the same or substantially similar amino acid sequence of a biologically active polypeptide.

The term “locus” refers to a specific location of a gene (or significant sequence), DNA sequence, polypeptide-encoding sequence, or position on a chromosome of the genome of an organism. For example, an “Tfrc locus” may refer to the specific location of an Tfrc gene, Tfrc DNA sequence, Tfrc-encoding sequence, or Tfrc position on a chromosome of the genome of an organism that has been identified as to where such a sequence resides. An “Tfrc locus” may comprise a regulatory element of an Tfrc gene, including, for example, an enhancer, a promoter, 5′ and/or 3′ untranslated region (UTR), or a combination thereof.

The term “gene” refers to a DNA sequence in a chromosome that codes for a product (e.g., an RNA product and/or a polypeptide product) and includes the coding region interrupted with non-coding introns and sequence located adjacent to the coding region on both the 5′ and 3′ ends such that the gene corresponds to the full-length mRNA (including the 5′ and 3′ untranslated sequences). The term “gene” also includes other non-coding sequences including regulatory sequences (e.g., promoters, enhancers, and transcription factor binding sites), polyadenylation signals, internal ribosome entry sites, silencers, insulating sequence, and matrix attachment regions. These sequences may be close to the coding region of the gene (e.g., within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The term “variant” refers to a nucleotide sequence differing from the sequence most prevalent in a population (e.g., by one nucleotide) or a protein sequence different from the sequence most prevalent in a population (e.g., by one amino acid).

The term “fragment” when referring to a protein means a protein that is shorter or has fewer amino acids than the full-length protein. The term “fragment” when referring to a nucleic acid means a nucleic acid that is shorter or has fewer nucleotides than the full-length nucleic acid. A fragment can be, for example, an N-terminal fragment (i.e., removal of a portion of the C-terminal end of the protein), a C-terminal fragment (i.e., removal of a portion of the N-terminal end of the protein), or an internal fragment.

“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “in vitro” includes artificial environments and to processes or reactions that occur within an artificial environment (e.g., a test tube). The term “in vivo” includes natural environments (e.g., a cell or organism or body) and to processes or reactions that occur within a natural environment. The term “ex vivo” includes cells that have been removed from the body of an individual and to processes or reactions that occur within such cells.

The term “reporter gene” refers to a nucleic acid having a sequence encoding a gene product (typically an enzyme) that is easily and quantifiably assayed when a construct comprising the reporter gene sequence operably linked to a heterologous promoter and/or enhancer element is introduced into cells containing (or which can be made to contain) the factors necessary for the activation of the promoter and/or enhancer elements. Examples of reporter genes include, but are not limited, to genes encoding beta-galactosidase (lacZ), the bacterial chloramphenicol acetyltransferase (cat) genes, firefly luciferase genes, genes encoding beta-glucuronidase (GUS), and genes encoding fluorescent proteins. A “reporter protein” refers to a protein encoded by a reporter gene.

The term “fluorescent reporter protein” as used herein means a reporter protein that is detectable based on fluorescence wherein the fluorescence may be either from the reporter protein directly, activity of the reporter protein on a fluorogenic substrate, or a protein with affinity for binding to a fluorescent tagged compound. Examples of fluorescent proteins include green fluorescent proteins (e.g., GFP, GFP-2, tagGFP, turboGFP, eGFP, Emerald, Azami Green, Monomeric Azami Green, CopGFP, AceGFP, and ZsGreen1), yellow fluorescent proteins (e.g., YFP, eYFP, Citrine, Venus, YPet, PhiYFP, and ZsYellow1), blue fluorescent proteins (e.g., BFP, eBFP, eBFP2, Azurite, mKalamal, GFPuv, Sapphire, and T-sapphire), cyan fluorescent proteins (e.g., CFP, eCFP, Cerulean, CyPet, AmCyanl, and Midoriishi-Cyan), red fluorescent proteins (e.g., RFP, mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, and Jred), orange fluorescent proteins (e.g., mOrange, mKO, Kusabira-Orange, Monomeric Kusabira-Orange, mTangerine, and tdTomato), and any other suitable fluorescent protein whose presence in cells can be detected by flow cytometry methods.

The term “recombination” includes any process of exchange of genetic information between two polynucleotides and can occur by any mechanism. Recombination in response to double-strand breaks (DSBs) occurs principally through two conserved DNA repair pathways: non-homologous end joining (NHEJ) and homologous recombination (HR). See Kasparek & Humphrey (2011) Seminars in Cell & Dev. Biol. 22:886-897, herein incorporated by reference in its entirety for all purposes. Likewise, repair of a target nucleic acid mediated by an exogenous donor nucleic acid can include any process of exchange of genetic information between the two polynucleotides.

NHEJ includes the repair of double-strand breaks in a nucleic acid by direct ligation of the break ends to one another or to an exogenous sequence without the need for a homologous template. Ligation of non-contiguous sequences by NHEJ can often result in deletions, insertions, or translocations near the site of the double-strand break. For example, NHEJ can also result in the targeted integration of an exogenous donor nucleic acid through direct ligation of the break ends with the ends of the exogenous donor nucleic acid (i.e., NHEJ-based capture). Such NHEJ-mediated targeted integration can be preferred for insertion of an exogenous donor nucleic acid when homology directed repair (HDR) pathways are not readily usable (e.g., in non-dividing cells, primary cells, and cells which perform homology-based DNA repair poorly). In addition, in contrast to homology-directed repair, knowledge concerning large regions of sequence identity flanking the cleavage site is not needed, which can be beneficial when attempting targeted insertion into organisms that have genomes for which there is limited knowledge of the genomic sequence. The integration can proceed via ligation of blunt ends between the exogenous donor nucleic acid and the cleaved genomic sequence, or via ligation of sticky ends (i.e., having 5′ or 3′ overhangs) using an exogenous donor nucleic acid that is flanked by overhangs that are compatible with those generated by a nuclease agent in the cleaved genomic sequence. See, e.g., US 2011/020722, WO 2014/033644, WO 2014/089290, and Maresca et al. (2013) Genome Res. 23(3):539-546, each of which is herein incorporated by reference in its entirety for all purposes. If blunt ends are ligated, target and/or donor resection may be needed to generation regions of microhomology needed for fragment joining, which may create unwanted alterations in the target sequence.

Recombination can also occur via homology directed repair (HDR) or homologous recombination (HR). HDR or HR includes a form of nucleic acid repair that can require nucleotide sequence homology, uses a “donor” molecule as a template for repair of a “target” molecule (i.e., the one that experienced the double-strand break), and leads to transfer of genetic information from the donor to target. Without wishing to be bound by any particular theory, such transfer can involve mismatch correction of heteroduplex DNA that forms between the broken target and the donor, and/or synthesis-dependent strand annealing, in which the donor is used to resynthesize genetic information that will become part of the target, and/or related processes. In some cases, the donor polynucleotide, a portion of the donor polynucleotide, a copy of the donor polynucleotide, or a portion of a copy of the donor polynucleotide integrates into the target DNA. See Wang et al. (2013) Cell 153:910-918; Mandalos et al. (2012) PLOS ONE 7:e45768:1-9; and Wang et al. (2013) Nat Biotechnol. 31:530-532, each of which is herein incorporated by reference in its entirety for all purposes.

The term “binding protein” includes any protein that binds to a cognate partner. The cognate partner of a binding protein is often referred to in the name of a binding protein, e.g., an “anti-X binding protein” refers to a protein that binds to “X”, where X refers to the name of an antigen. Examples of binding proteins include an antibody, a fragment of an antibody that binds the cognate partner, a multispecific antibody (e.g., a bi-specific antibody), an scFV, a bis-scFV, a diabody, a triabody, a tetrabody, a V-NAR, a VHH, a VL, a F(ab), a F(ab)₂, a DVD (dual variable domain binding protein), an SVD (single variable domain binding protein), a bispecific T-cell engager (BiTE), or a Davisbody (U.S. Pat. No. 8,586,713, herein incorporated by reference herein in its entirety for all purposes). Example binding proteins that bind TfR, e.g., anti-TfR antibodies (including anti-hTfR antibodies) include, but are not limited to, anti-transferrin receptor antibodies; see, e.g., US20170174778; US20150196663; U.S. Pat. No. 9,629,801; US20180002433; WO2016081643; US20180134797; WO2014189973; US20150110791; U.S. Pat. No. 9,708,406; US20170260292; WO2016081640; US20180057604; U.S. Pat. No. 9,611,323; WO2012075037; WO2018210898, US20180344869, US20180282408, US20170051071, WO2016207240, WO2015101588, US20160324984; US20180222993; WO2017055542; US20180222992; WO2017055540; Cabezon, I., et al. Mol Pharm. 2015 Nov. 2; 12(11):4137-45; Yu Y J, et al. Sci Transl Med (2014) 6:261ra154; Couch, et al. Sci Transl Med. 2013 May 1; 5(183):183ra57, 1-12; CH3 domains that are mutated to specifically bind TfR, see, e.g., WO2023114499, WO2019032955, WO2018152326, WO2019140050, WO2019140050; inter alia.

The term “multi-specific” or “bi-specific” with reference to a binding protein means that the protein recognizes different epitopes, either on the same antigen or on different antigens. A multi-specific binding protein can be a single multifunctional polypeptide, or it can be a multimeric complex of two or more polypeptides that are covalently or non-covalently associated with one another. For example, an antibody or fragment thereof can be functionally linked (e.g., by chemical coupling, genetic fusion, non-covalent association or otherwise) to one or more other molecular entities, such as a protein or fragment thereof to produce a bispecific or a multi-specific binding molecule with a second binding specificity.

The term “antigen” refers to a substance, whether an entire molecule or a domain within a molecule, which is capable of eliciting production of antibodies or other cognate binding proteins with binding specificity to that substance. The term antigen also includes substances, which in wild type host organisms would not elicit antibody or other cognate binding proteins production by virtue of self-recognition, but can elicit such a response in a host animal with appropriate genetic engineering to break immunological tolerance.

The term “epitope” refers to a site on an antigen to which a binding protein (e.g., antibody) binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996), herein incorporated by reference in its entirety for all purposes.

An “antibody paratope” as described herein generally comprises at a minimum a complementarity determining region (CDR) that specifically recognizes the heterologous epitope (e.g., a CDR3 region of a heavy and/or light chain variable domain).

The term “antibody” includes immunoglobulin molecules comprising four polypeptide chains, two heavy (H) chains and two light (L) chains inter-connected by disulfide bonds. Each heavy chain comprises a heavy chain variable domain and a heavy chain constant region (C_{H}). The heavy chain constant region comprises three domains: C_{H}1, C_{H}2 and C_{H}3. Each light chain comprises a light chain variable domain and a light chain constant region (CL). The heavy chain and light chain variable domains can be further subdivided into regions of hypervariability, termed complementarity determining regions (CDR), interspersed with regions that are more conserved, termed framework regions (FR). Each heavy and light chain variable domain comprises three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3). The term “high affinity” antibody refers to an antibody that has a K_{D} with respect to its target epitope about of 10⁻⁹ M or lower (e.g., about 1×10⁻⁹ M, 1×10⁻¹⁰ M, 1×10⁻¹¹ M, or about 1×10⁻¹ M). In one embodiment, K_{D} is measured by surface plasmon resonance, e.g., BIACORE™; in another embodiment, K_{D} is measured by ELISA.

The term “bispecific antibody” includes an antibody capable of selectively binding two or more epitopes. Bispecific antibodies generally comprise two different heavy chains, with each heavy chain specifically binding a different epitope-either on two different molecules (e.g., on two different antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer binding specificity but that can associate with each heavy chain, or that can associate with each heavy chain and that can bind one or more of the epitopes bound by the heavy chain binding regions, or that can associate with each heavy chain and enable binding or one or both of the heavy chains to one or both epitopes.

The term “heavy chain,” or “immunoglobulin heavy chain” includes an immunoglobulin heavy chain sequence, including immunoglobulin heavy chain constant region sequence, from any organism. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a C_{H}1 domain, a hinge, a C_{H}2 domain, and a C_{H}3 domain. A functional fragment of a heavy chain includes a fragment that is capable of specifically recognizing an epitope (e.g., recognizing the epitope with a K_{D} in the micromolar, nanomolar, or picomolar range), that is capable of expressing and secreting from a cell, and that comprises at least one CDR. Heavy chain variable domains are encoded by variable region nucleotide sequence, which generally comprises V_{H}, D_{H}, and J_{H} segments derived from a repertoire of V_{H}, D_{H}, and J_{H} segments present in the germline. Sequences, locations and nomenclature for V, D, and J heavy chain segments for various organisms can be found in IMGT database, which is accessible via the internet on the World Wide Web (www) at the URL “imgt.org.”

The term “light chain” includes an immunoglobulin light chain sequence from any organism, and unless otherwise specified includes human kappa (x) and lambda (k) light chains and a VpreB, as well as surrogate light chains. Light chain variable domains typically include three light chain CDRs and four framework (FR) regions, unless otherwise specified. Generally, a full-length light chain includes, from amino terminus to carboxyl terminus, a variable domain that includes FR1-CDR1-FR2-CDR2-FR3-CDR3-FR4, and a light chain constant region amino acid sequence. Light chain variable domains are encoded by the light chain variable region nucleotide sequence, which generally comprises light chain VL and light chain J_{L} gene segments, derived from a repertoire of light chain V and J gene segments present in the germline. Sequences, locations and nomenclature for light chain V and J gene segments for various organisms can be found in IMGT database, which is accessible via the internet on the World Wide Web (www) at the URL “imgt.org.” Light chains include those, e.g., that do not selectively bind either a first or a second epitope selectively bound by the epitope-binding protein in which they appear. Light chains also include those that bind and recognize, or assist the heavy chain with binding and recognizing, one or more epitopes selectively bound by the epitope-binding protein in which they appear.

The term “complementary determining region” or “CDR,” as used herein, includes an amino acid sequence encoded by a nucleic acid sequence of an organism's immunoglobulin genes that normally (i.e., in a wild type animal) appears between two framework regions in a variable region of a light or a heavy chain of an immunoglobulin molecule (e.g., an antibody or a T cell receptor). A CDR can be encoded by, for example, a germline sequence or a rearranged sequence, and, for example, by a naïve or a mature B cell or a T cell. A CDR can be somatically mutated (e.g., vary from a sequence encoded in an animal's germline), humanized, and/or modified with amino acid substitutions, additions, or deletions. In some circumstances (e.g., for a CDR3), CDRs can be encoded by two or more sequences (e.g., germline sequences) that are not contiguous (e.g., in an unrearranged nucleic acid sequence) but are contiguous in a B cell nucleic acid sequence, e.g., as a result of splicing or connecting the sequences (e.g., V-D-J recombination to form a heavy chain CDR3.

Specific binding of a binding protein to its target antigen includes binding with an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas non-specific binding is usually the result of van der Waals forces. Specific binding does not however necessarily imply that an binding protein binds one and only one target.

“Optional” or “optionally” means that the subsequently described event or circumstance may or may not occur and that the description includes instances in which the event or circumstance occurs and instances in which it does not.

Designation of a range of values includes all integers within or defining the range, and all subranges defined by integers within the range.

Unless otherwise apparent from the context, the term “about” encompasses values within a standard margin of error of measurement (e.g., SEM) of a stated value.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

### II. Non-Human Cells and Non-Human Animals Comprising a Humanized Tfrc Locus

Non-human animal cells, non-human animal genomes (e.g., non-human animal cell nuclei), and non-human animals comprising a human or humanized Tfrc locus described herein as described herein are provided. The cells, genomes (nuclei), or non-human animals can be heterozygous or homozygous for the humanized Tfrc locus. A diploid organism has two alleles at each genetic locus. Each pair of alleles represents the genotype of a specific genetic locus. Genotypes are described as homozygous if there are two identical alleles at a particular locus and as heterozygous if the two alleles differ. In some embodiments, provided herein is a non-human animal cell or genome (cell nuclei) comprising a genetically modified endogenous Tfrc locus encoding a modified TfR protein, wherein the modified TfR protein comprises a domain of a human TFR sequence, and all or part of the domain is encoded by a segment of the endogenous Tfrc locus that has been deleted and replaced with an orthologous human TFRC sequence.

The non-human animal cell can be a blood brain barrier (BBB) endothelial cell, a pluripotent cell, an ES cell, or a germ cell.

In some embodiments, the disclosure further provides methods for making any non-human animal, or reagents required for making the non-human animal as described herein.

The non-human animal cells and genomes (e.g., nuclei) provided herein can be, for example, any non-human cell comprising a Tfrc locus or a genomic Tfrc locus that is homologous or orthologous to the human TFRC locus. The cells and genomes (e.g., nuclei) can be eukaryotic, which include, for example, fungal (e.g., yeast) cells and genomes, plant cells and genomes, animal cells and genomes, mammalian cells and genomes, non-human mammalian cells and genomes, etc. A non-human animal can be, for example, a mammal, fish, or bird. A mammalian cell can be, for example, a non-human mammalian cell, a rodent cell, a rat cell, a mouse cell, or a hamster cell. Other non-human mammals include, for example, non-human primates, monkeys, apes, orangutans, cats, dogs, rabbits, horses, bulls, deer, bison, livestock (e.g., bovine species such as cows, steer, and so forth; ovine species such as sheep, goats, and so forth; and porcine species such as pigs and boars). Birds include, for example, chickens, turkeys, ostrich, geese, ducks, and so forth. Domesticated animals and agricultural animals are also included. The term “non-human” excludes humans.

The cells can also be any type of undifferentiated or differentiated state. For example, a cell can be a totipotent cell, a pluripotent cell (e.g., a human pluripotent cell or a non-human pluripotent cell such as a mouse embryonic stem (ES) cell or a rat ES cell), or a non-pluripotent cell. Totipotent cells include undifferentiated cells that can give rise to any cell type, and pluripotent cells include undifferentiated cells that possess the ability to develop into more than one differentiated cell types. Such pluripotent and/or totipotent cells can be, for example, ES cells or ES-like cells, such as an induced pluripotent stem (iPS) cells. ES cells include embryo-derived totipotent or pluripotent cells that can contribute to any tissue of the developing embryo upon introduction into an embryo. ES cells can be derived from the inner cell mass of a blastocyst and can differentiate into cells of any of the three vertebrate germ layers (endoderm, ectoderm, and mesoderm).

The cells provided herein can also be germ cells (e.g., sperm or oocytes). The cells can be mitotically competent cells or mitotically-inactive cells, meiotically competent cells or meiotically-inactive cells. Similarly, the cells disclosed herein can also be primary somatic cells or cells that are not a primary somatic cell. Somatic cells include any cell that is not a gamete, germ cell, gametocyte, or undifferentiated stem cell. Suitable cells provided herein also include primary cells. Primary cells include cells or cultures of cells that have been isolated directly from an organism, organ, or tissue. Primary cells include cells that are neither transformed nor immortal. Primary cells include any cell obtained from an organism, organ, or tissue which was not previously passed in tissue culture or has been previously passed in tissue culture but is incapable of being indefinitely passed in tissue culture. Such cells can be isolated by conventional techniques. Table 1 provides a non-limiting list of tissues and associated cells that may express the heterologous TfR protein as described herein. Any one of or any combination of non-human animal tissues/cell types listed in Table 1 may express a heterologous TfR protein.

TABLE 1

| Target tissue | Cell types |
|---|---|
| Brain/Spinal | endothelial cells |
| cord/CNS/BBB | neurons (all types) |
| | oligodendrocytes (and precursors) |
| | pericytes |
| | meninges/leptomeningeal cells |
| | arachnoid barrier cells |
| | peripheral glia |
| | astrocytes |
| | glia |
| | schwann cells |
| | ependymal cells |
| | microglia |
| Eye | rod photoreceptor cells |
| | muller glia cells |
| | bipolar cells |
| | cone photoreceptor cells |
| | endothelial cells |
| | cornea |
| | sclera |
| | optic nerve |
| | pupillary sphincter |
| Skeletal Muscle | skeletal myocytes |
| | fibroblasts |
| | endothelial cells |
| | macrophages |
| | satellite cells |
| Adipose tissue | Adipocytes |
| | fibroblasts |
| | T-cells |
| | Macrophages |
| | B-cells |
| | Dendritic cells |
| Blood/Bone | T-cells |
| marrow | B-cells |
| | Macrophages |
| | erythroid cells |
| | plasma cells |
| | dendritic cells |
| Breast | glandular cells |
| | T-cells |
| | fibroblasts |
| | macrophages |
| | endothelial cells |
| | myoepithelial cells |
| | Adipocytes |
| Lung/Bronchus | Basal respiratory cells |
| | respiratory cilliated cells |
| | club cells |
| | smooth muscle cells |
| | ionocytes |
| | macrophages |
| | alveolar cells (type 1 & 2) |
| | T-cells |
| | enothelial cells |
| Colon | distal enterocytes |
| | intestinal goblet cells |
| | undifferentiated cells |
| | T-cells |
| | paneth cells |
| | B-cells |
| | enteroednocrine cells |
| Uterus | glandular and luminal cells |
| | endometrial stromal cells |
| | endothelial cells |
| | smooth muscle cells |
| | T-cells |
| | macrophages |
| Esophagus | fibroblasts |
| | squamous epithelial cells |
| | endothelial cells |
| | smooth muscle cells |
| | macrophages |
| | plasma cells |
| | T-cells |
| Heart | cardiomyocytes |
| | endothelial cells |
| | fibroblasts |
| | macrophages |
| | T-cells |
| | B-cells |
| | Dendritic cells |
| Kidney | proximal tubular cells |
| | T-cells |
| | macrophages |
| | collecting duct cells |
| | B-cells |
| | glomeruli |
| | fibroblasts |
| Liver | hepatocytes |
| | B-cells |
| | erythroid cells |
| Lymph node | B-cells |
| | T-cells |
| Ovary | granulosa cells |
| | fibroblasts |
| | smooth muscle cells |
| | macrophages |
| | T-cells |
| | theca cells |
| | fibroblasts |
| Pancreas | ductal cells |
| | pancreatic endocrine cells |
| | smooth muscle cells |
| | endothelial cells |
| | macrophages |
| | exocrine glandular cells |
| | monocytes |
| Placenta | cytotrophoblasts |
| | extravillous trophoblasts |
| | fibroblasts |
| | hofbauer cells |
| | endothelial cells |
| Prostate | basal prostatic cells |
| | prostatic glandular cells |
| | urothelial cells |
| | endothelial cells |
| | fibroblasts |
| | smooth muscle cells |
| | macrophages |
| | T-cells |
| Rectum | undifferentiated cells |
| | intestinal goblet cells |
| | paneth cells |
| | distal enterocytes |
| | enteroednocrine cells |
| Skin | langerhans cells |
| | fibroblasts |
| | endothelial cells |
| | basal keratinocytes |
| | suprabasal keratinocytes |
| | T-cells |
| | smooth muscle cells |
| | melanocytes |
| PBMC | monocytes |
| | T-cells |
| | NK-cells |
| | dendritic cells |
| Small intestine | proximal enterocytes |
| | undifferentiated cells |
| | intestinal goblet cells |
| | paneth cells |
| Spleen | B-cells |
| | T-cells |
| | plasma cells |
| | macrophages |
| Stomach | B-cells |
| | T-cells |
| | gastric mucus-secreting cells |
| | plasma cells |
| | fibroblasts |
| | macrophages |
| Testis | leydig cells |
| | late spermatids |
| | spermatogonia |
| | early spermatids |
| | macrophages |
| | spermatocytes |
| | peritubular cells |
| | sertoli cells |
| | endothelial cells |
| Peripheral nervous | motor neurons |
| system | sensory neurons |
| | schwann cells |
| | dorsal root ganglion |
| Bone/cartilage/joint | chondrocytes |
| | chondroblasts |
| | mesenchymal cells |
| | osteoblasts |
| | osteoclasts |

Other suitable cells provided herein include immortalized cells. Immortalized cells include cells from a multicellular organism that would normally not proliferate indefinitely but, due to mutation or alteration, have evaded normal cellular senescence and instead can keep undergoing division. Such mutations or alterations can occur naturally or be intentionally induced. Examples of immortalized cell lines are myofiber cell lines. Immortalized or primary cells include cells that can be used for culturing or for expressing recombinant genes or proteins.

The cells provided herein also include one-cell stage embryos (i.e., fertilized oocytes or zygotes). Such one-cell stage embryos can be from any genetic background (e.g., BALB/c, C57BL/6, 129, or a combination thereof for mice), can be fresh or frozen, and can be derived from natural breeding or in vitro fertilization.

The cells provided herein can be normal, healthy cells, or can be diseased or mutant-bearing cells.

Non-human animals comprising a humanized Tfrc locus as described herein can be made by the methods described elsewhere herein. An animal can be, for example, a mammal, fish, or bird. Non-human mammals include, for example, non-human primates, monkeys, apes, orangutans, cats, dogs, horses, bulls, deer, bison, sheep, rabbits, rodents (e.g., mice, rats, hamsters, and guinea pigs), and livestock (e.g., bovine species such as cows and steer; ovine species such as sheep and goats; and porcine species such as pigs and boars). Birds include, for example, chickens, turkeys, ostrich, geese, and ducks. Domesticated animals and agricultural animals are also included. The term “non-human animal” excludes humans. Preferred non-human animals include, for example, rodents, such as mice and rats.

The non-human animals can be from any genetic background. For example, suitable mice can be from a 129 strain, a C57BL/6 strain, a mix of 129 and C57BL/6, a BALB/c strain, or a Swiss Webster strain. Examples of 129 strains include 129P1, 129P2, 129P3, 129X1, 129S1 (e.g., 129S1/SV, 129S1/Svlm), 129S2, 129S4, 129S5, 12959/SvEvH, 129S6 (129/SvEvTac), 129S7, 129S8, 129T1, and 129T2. See, e.g., Festing et al. (1999) Mammalian Genome 10:836, herein incorporated by reference in its entirety for all purposes. Examples of C57BL strains include C57BL/A, C57BL/An, C57BL/GrFa, C57BL/Ka1_wN, C57BL/6, C57BL/6J, C57BL/6ByJ, C57BL/6NJ, C57BL/10, C57BL/10ScSn, C57BL/10Cr, and C57BL/Ola. Suitable mice can also be from a mix of an aforementioned 129 strain and an aforementioned C57BL/6 strain (e.g., 50% 129 and 50% C57BL/6). Likewise, suitable mice can be from a mix of aforementioned 129 strains or a mix of aforementioned BL/6 strains (e.g., the 129S6 (129/SvEvTac) strain).

Similarly, rats can be from any rat strain, including, for example, an ACI rat strain, a Dark Agouti (DA) rat strain, a Wistar rat strain, a LEA rat strain, a Sprague Dawley (SD) rat strain, or a Fischer rat strain such as Fisher F344 or Fisher F6. Rats can also be obtained from a strain derived from a mix of two or more strains recited above. For example, a suitable rat can be from a DA strain or an ACI strain. The ACI rat strain is characterized as having black agouti, with white belly and feet and an RT1^{avl} haplotype. Such strains are available from a variety of sources including Harlan Laboratories. The Dark Agouti (DA) rat strain is characterized as having an agouti coat and an RT1^{avl} haplotype. Such rats are available from a variety of sources including Charles River and Harlan Laboratories. Some suitable rats can be from an inbred rat strain. See, e.g., US 2014/0235933, herein incorporated by reference in its entirety for all purposes.

### II. Methods of Making Non-Human Animals Comprising a Heterologous Tfrc Locus

Various methods are provided for making a non-human animal comprising a heterologous Tfrc locus as disclosed elsewhere herein. Any convenient method or protocol for producing a genetically modified organism is suitable for producing such a genetically modified non-human animal. See, e.g., Cho et al. (2009) Current Protocols in Cell Biology 42:19.11:19.11.1-19.11.22 and Gama Sosa et al. (2010) Brain Struct. Funct. 214(2-3):91-109, each of which is herein incorporated by reference in its entirety for all purposes. Such genetically modified non-human animals can be generated, for example, through gene knock-in at a targeted Tfrc locus.

For example, the method of producing a non-human animal comprising a humanized Tfrc locus can comprise: (1) modifying the genome of a pluripotent cell to comprise the humanized Tfrc locus; (2) identifying or selecting the genetically modified pluripotent cell comprising the humanized Tfrc locus; (3) introducing the genetically modified pluripotent cell into a non-human animal host embryo cells in vitro; and (4) implanting and gestating the host embryo cells in a surrogate mother. Optionally, the host embryo comprising modified pluripotent cell (e.g., a non-human ES cell) can be incubated until the blastocyst stage before being implanted into and gestated in the surrogate mother to produce an F0 non-human animal. The surrogate mother can then produce an F0 generation non-human animal comprising the humanized Tfrc locus.

The methods can further comprise identifying a cell or animal having a modified target genomic locus. Various methods can be used to identify cells and animals having a targeted genetic modification.

The screening step can comprise, for example, a quantitative assay for assessing modification of allele (MOA) of a parental chromosome. For example, the quantitative assay can be carried out via a quantitative PCR, such as a real-time PCR (qPCR). The real-time PCR can utilize a first primer set that recognizes the target locus and a second primer set that recognizes a non-targeted reference locus. The primer set can comprise a fluorescent probe that recognizes the amplified sequence.

Other examples of suitable quantitative assays include fluorescence-mediated in situ hybridization (FISH), comparative genomic hybridization, isothermic DNA amplification, quantitative hybridization to an immobilized probe(s), INVADER® Probes, TAQMAN® Molecular Beacon probes, or ECLIPSE™ probe technology (see, e.g., US 2005/0144655, incorporated herein by reference in its entirety for all purposes).

An example of a suitable pluripotent cell is an embryonic stem (ES) cell (e.g., a mouse ES cell or a rat ES cell). The modified pluripotent cell can be generated, for example, through recombination by (a) introducing into the cell one or more targeting vectors comprising an insert nucleic acid flanked by 5′ and 3′ homology arms corresponding to 5′ and 3′ target sites, wherein the insert nucleic acid comprises a heterologous Tfrc locus; and (b) identifying at least one cell comprising in its genome the insert nucleic acid integrated at the target genomic locus. Alternatively, the modified pluripotent cell can be generated by (a) introducing into the cell: (i) a nuclease agent, wherein the nuclease agent induces a nick or double-strand break at a recognition site within the target genomic locus; and (ii) one or more targeting vectors comprising an insert nucleic acid flanked by 5′ and 3′ homology arms corresponding to 5′ and 3′ target sites located in sufficient proximity to the recognition site, wherein the insert nucleic acid comprises the heterologous Tfrc locus; and (c) identifying at least one cell comprising a modification (e.g., integration of the insert nucleic acid) at the target genomic locus. Any nuclease agent that induces a nick or double-strand break into a desired recognition site can be used. Examples of suitable nucleases include a Transcription Activator-Like Effector Nuclease (TALEN), a zinc-finger nuclease (ZFN), a meganuclease, and Clustered Regularly Interspersed Short Palindromic Repeats (CRISPR)/CRISPR-associated (Cas) systems or components of such systems (e.g., CRISPR/Cas9). See, e.g., US 2013/0309670 and US 2015/0159175, each of which is herein incorporated by reference in its entirety for all purposes.

The donor cell can be introduced into a host embryo at any stage, such as the blastocyst stage or the pre-morula stage (i.e., the 4 cell stage or the 8 cell stage). Progeny that are capable of transmitting the genetic modification though the germline are generated. See, e.g., U.S. Pat. No. 7,294,754, herein incorporated by reference in its entirety for all purposes.

Alternatively, the method of producing the non-human animals described elsewhere herein can comprise: (1) modifying the genome of a one-cell stage embryo to comprise the heterologous Tfrc locus using the methods described above for modifying pluripotent cells; (2) selecting the genetically modified embryo; and (3) implanting and gestating the genetically modified embryo into a surrogate mother. Progeny that are capable of transmitting the genetic modification though the germline are generated.

Nuclear transfer techniques can also be used to generate the non-human mammalian animals. Briefly, methods for nuclear transfer can include the steps of: (1) enucleating an oocyte or providing an enucleated oocyte; (2) isolating or providing a donor cell or nucleus to be combined with the enucleated oocyte; (3) inserting the cell or nucleus into the enucleated oocyte to form a reconstituted cell; (4) implanting the reconstituted cell into the womb of an animal to form an embryo; and (5) allowing the embryo to develop. In such methods, oocytes are generally retrieved from deceased animals, although they may be isolated also from either oviducts and/or ovaries of live animals. Insertion of the donor cell or nucleus into the enucleated oocyte to form a reconstituted cell can be by microinjection of a donor cell under the zona pellucida prior to fusion. Fusion may be induced by application of a DC electrical pulse across the contact/fusion plane (electrofusion), by exposure of the cells to fusion-promoting chemicals, such as polyethylene glycol, or by way of an inactivated virus, such as the Sendai virus. A reconstituted cell can be activated by electrical and/or non-electrical means before, during, and/or after fusion of the nuclear donor and recipient oocyte. Activation methods include electric pulses, chemically induced shock, penetration by sperm, increasing levels of divalent cations in the oocyte, and reducing phosphorylation of cellular proteins (as by way of kinase inhibitors) in the oocyte. The activated reconstituted cells, or embryos, can be cultured in media and then transferred to the womb of an animal. See, e.g., US 2008/0092249, WO 1999/005266, US 2004/0177390, WO 2008/017234, and U.S. Pat. No. 7,612,250, each of which is herein incorporated by reference in its entirety for all purposes.

The various methods provided herein allow for the generation of a genetically modified non-human F0 animal wherein the cells of the genetically modified F0 animal comprise the humanized Tfrc locus. It is recognized that depending on the method used to generate the F0 animal, the number of cells within the F0 animal that have the heterologous Tfrc locus will vary. The introduction of the donor ES cells into a pre-morula stage embryo from a corresponding organism (e.g., an 8-cell stage mouse embryo) via for example, the VELOCIMOUSE® method allows for a greater percentage of the cell population of the F0 animal to comprise cells having the nucleotide sequence of interest comprising the targeted genetic modification. For example, at least 50%, 60%, 65%, 70%, 75%, 85%, 86%, 87%, 87%, 88%, 89%, 90%, 91%, 92%, 93%, 94%, 95%, 96%, 97%, 98%, 99% or 100% of the cellular contribution of the non-human F0 animal can comprise a cell population having the targeted modification.

The cells of the genetically modified F0 animal can be heterozygous for the heterologous Tfrc locus. In some embodiment heterozygous F0 mice may be bred to generate progeny that are homozygous for the heterologous Tfrc locus.

In some embodiments, the disclosure provides a method of making a non-human animal, a non-human animal cell, or a non-human animal genome of described herein, comprising inserting a nucleic acid sequence encoding the heterologous TfR protein or portion thereof into the genome of the non-human animal, the genome of the non-human animal cell, or the non-human animal genome.

A variety of nucleic acids (e.g., targeting vectors) can be specifically used for such purposes. In some embodiments, a nucleic acid molecule that encodes a functional TfR protein comprises a nucleic acid sequence of a modified non-human animal Tfrc gene, wherein the modified non-human animal Tfrc gene comprises a replacement of a nucleic sequence encoding a portion of the non-human animal TfR protein with a homologous nucleic acid sequence encoding a heterologous TfR protein or portion thereof can be used in the genetic editing of a cell or genome described herein. In some cases, such nucleic acid molecules (e.g., targeting molecules may comprise: (i) a nucleic acid sequence comprising exon 1 of a human TFRC gene or a portion thereof; (ii) a nucleic acid sequence comprising exon 2 of a human TFRC gene or a portion thereof; (iii) a nucleic acid sequence comprising exon 3 of a human TFRC gene or a portion thereof; (iv) a nucleic acid sequence comprising exon 4 of a human TFRC gene or a portion thereof, or (v) a nucleic acid sequence comprising exon 5 of a human TFRC gene or a portion thereof; (vi) a nucleic acid sequence comprising exon 6 of a human TFRC gene or a portion thereof; (vii) a nucleic acid sequence comprising exon 7 of a human TFRC gene or a portion thereof; (viii) a nucleic acid sequence comprising exon 8 of a human TFRC gene, (ix) a nucleic acid sequence comprising exon 9 of a human TFRC gene or a portion thereof; (x) a nucleic acid sequence comprising exon 10 of a human TFRC gene or a portion thereof; (xi) a nucleic acid sequence comprising exon 11 of a human TFRC gene or a portion thereof; (xii) a nucleic acid sequence comprising exon 12 of a human TFRC gene or a portion thereof or a portion thereof, (xiii) a nucleic acid sequence comprising exon 13 of a human TFRC gene or a portion thereof; (xiv) a nucleic acid sequence comprising exon 14 of a human TFRC gene or a portion thereof; (xv) a nucleic acid sequence comprising exon 15 of a human TFRC gene or a portion thereof; (xvi) a nucleic acid sequence comprising exon 16 of a human TFRC gene or a portion thereof, (xvii) a nucleic acid sequence comprising exon 17 of a human TFRC gene or a portion thereof; (xviii) a nucleic acid sequence comprising exon 18 of a human TFRC gene or a portion thereof; (ixx) a nucleic acid sequence comprising exon 19 of a human TFRC gene or a portion thereof; (xx) or any combination of (i)-(ixx), and optionally any introns between any included exons or portion thereof. In some embodiments, nucleic acid sequences encoding a heterologous TfR protein or portion thereof may comprise a nucleic acid sequence set forth in exon 3, 4, 5, 6, 7, 8, 9, 10, 11, 12, 13, 14, 15, 16, 17, 18, and 19 (or a coding portion of exon 19) of a human TFRC gene, an optionally any introns between such exons.

In some embodiments, a nucleic acid molecule (e.g., targeting vector) described herein comprises (i) a 5′ homology arm upstream of the modified non-human animal Tfrc gene and (ii) a 3′ homology arm downstream of the modified non-human animal Tfrc gene. In some embodiments, the 5′ homology arm and 3′ homology arm are configured to undergo homologous recombination with a non-human animal Tfrc locus of interest, and following homologous recombination with a non-human animal Tfrc locus of interest, the modified Tfrc gene replaces the non-human animal Tfrc gene at the non-human animal Tfrc locus of interest and is operably linked to an endogenous promoter that drives expression of the modified non-human animal Tfrc gene at the non-human animal Tfrc locus of interest. In some embodiments, the nucleic acid molecule (e.g., targeting vectors) comprises a nucleic acid sequence set forth as SEQ ID NO:5, a nucleic acid sequence set forth as SEQ ID NO:6, a nucleic acid sequence set forth as SEQ ID NO:9, or a nucleic acid sequence set forth as SEQ ID NO:10.

### IV. Mouse Model for Testing Human Therapies/Methods of Using Non-Human Animals Comprising a Heterologous Tfrc Locus

The non-human animals and cells described herein may be useful as models for preclinical testing of TfR-based therapy modalities, e.g., use of TfR as an internalizing effector for the intracellularization of therapeutic agents and/or as “molecular trojan horse” to ferry macromolecules across the blood brain barrier in a human. See, e.g., WO2013/138400, WO2017/007796, WO2018/226861, WO2018/226861, WO2019/157224, each of which is incorporated in its entirety by reference; see also Pardridge (2007) J. Control Release 122(3):345-348.

In some embodiments, described herein are non-human animal models for testing anti-human (h)-TfR binding proteins, e.g., anti-human (h)-TfR antibodies. In some such embodiments, described is a non-human animal model for testing a multidomain therapeutic comprising an anti-human (h)-TfR binding protein fused with a therapeutic agent. As such, tested anti-human (h)-TfR binding proteins as described herein (including an a multidomain therapeutic comprising an anti-human (h)-TfR binding protein fused with a therapeutic agent) targets or specifically binds a human (h) TfR protein or portion thereof displayed in a non-human animal or by a non-human animal cell as described herein, and the intracellularization of the anti-human (h)-TfR binding protein by non-human animal cells that express the human TfR protein or portion thereof and/or transport of the anti-human-TfR binding proteins across the blood brain barrier may be monitored to evaluate the efficacy of the anti-human-TfR binding protein (or a multidomain therapeutic comprising an anti-human-TfR binding protein fused with a therapeutic agent).

In some embodiments, the testing of the anti-human-TfR binding protein involves performing an assay or a study that allows determination of the effect of the anti-human-TfR binding proteins on a cell expressing the human TfR protein or a portion thereof. In some embodiments, the determination of the effect of the anti-human-TfR binding protein comprises measuring the intracellular level of the anti-human-TfR binding protein (and/or therapeutic agent that was fused or carried by the anti-human-TfR binding protein). In some embodiments, the determination of the effect of the anti-human-TfR binding protein comprises measuring the level of the anti-human-TfR binding protein (and/or therapeutic agent that was fused or carried by the anti-human-TfR binding protein), e.g., in the central nervous system, particularly if the anti-human-TfR binding protein (and/or therapeutic agent that was fused or carried by the anti-human-TfR binding protein) was administered to the non-human animal parenterally and/or via intravenous injection.

In some embodiments, the determination of the effect of the anti-human-TfR antigen protein comprises administering, in a non-human animal that is genetically modified to express a human TfR protein as described herein and further modified to exhibit one or more symptoms of a human disease, a candidate human-TfR binding protein (e.g., a multidomain therapeutic comprising an anti-human-TfR binding protein fused with a therapeutic agent) and evaluating the efficacy of the candidate human-TfR binding protein fused to a therapeutic agent to reduce, prevent, reduce the likelihood of, and/or inhibit the one or more symptoms of a human disease.

In some embodiments, in such a non-human animal model, the anti-human-TfR binding protein (e.g., a multidomain therapeutic comprising an anti-human-TfR binding protein fused with a therapeutic agent) may be introduced into a non-human animal as described herein. The anti-human-TfR binding protein (e.g., a multidomain therapeutic comprising an anti-human-TfR binding protein fused with a therapeutic agent), or a gene encoding the anti-human-TfR binding protein, may be introduced into a non-human animal as described herein by several methods known to those skilled in the art. Some nonlimiting methods include transgenesis, hydrodynamic delivery (HDD), lipid nanoparticle (LNP) delivery, intravenous injection, parenteral administration, tissue or cell transplantation, etc. Nucleotides encoding anti-human-TfR binding protein (e.g., a multidomain therapeutic comprising an anti-human-TfR binding protein fused with a therapeutic agent) may be targeted to be expressed by particular cell types, e.g., the liver, or to be expressed by a particular locus, e.g., a safe harbor locus, according to well-known methods. As a non-limiting example, when administering nucleotides encoding an anti-human-TfR binding protein by LNP delivery, the LNP may contain one or more or all of the following: (i) a lipid for encapsulation and for endosomal escape; (ii) a neutral lipid for stabilization; (iii) a helper lipid for stabilization; and (iv) a stealth lipid. In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA. In certain LNPs, the cargo can include an exogenous donor sequence (e.g., encoding the anti-human-TfR binding protein). In certain LNPs, the cargo can include a nuclease agent (or a nucleic acid encoding the nuclease agent or one or more nucleic acids encoding the nuclease agent) and an exogenous donor sequence (e.g., encoding the anti-human-TfR binding protein). In certain LNPs, the cargo can include an mRNA encoding a Cas nuclease, such as Cas9, a guide RNA or a nucleic acid encoding a guide RNA, and an exogenous donor sequence (e.g., encoding the anti-human-TfR binding protein) for CRISPR-mediated insertion of the exogenous donor sequence (e.g., encoding the anti-human-TfR binding protein) into a safe harbor locus of the animal, such as but not limited to a safe harbor locus, e.g., albumin, e.g., the first intron of the albumin locus. See, e.g., WO2020206162, incorporated herein in its entirety by reference.

In some embodiments, a non-human animal as described herein may be used as a model to determine the efficacy of using an anti-human-TfR binding protein to deliver a therapeutic protein to a tissue or cell listed in Table 1, e.g., the central nervous system (CNS), of a subject, comprising administering to the non-human animal an anti-human-TfR binding protein (e.g., a multidomain therapeutic comprising an anti-human-TfR binding protein fused with a therapeutic agent) or a nucleotide composition encoding the same, and then measuring the levels of the anti-human-TfR binding protein (e.g., a multidomain therapeutic comprising an anti-human-TfR binding protein fused with a therapeutic agent) or the therapeutic agent in the tissue or cell listed in Table 1, e.g., the CNS, of the subject. Methods of measuring the levels of the anti-human-TfR binding protein (e.g., a multidomain therapeutic comprising an anti-human-TfR binding protein fused with a therapeutic agent) are well-known in the art and include, but are not limited to, Western Blotting, enzyme-linked immunoassays, etc.

Certain disorders may benefit from pre-clinical testing of an anti-human-TfR binding protein (e.g., a multidomain therapeutic comprising an anti-human-TfR binding protein fused with a therapeutic agent), particularly as a candidate for shuttling a therapeutic agent across the cell membrane (e.g., into lysosomes) and/or transport of a therapeutic agent across the blood brain barrier. Table 2 provides a non-limiting list of disorders that may benefit from the use of an anti-TfR antibody for TfR-mediated intracellularization of a therapeutic agent to an organelle or tissue and/or TfR-mediated crossing of the blood brain barrier by the therapeutic agent. Accordingly, in some embodiments, a non-human animal as described herein provides a drug screening platform in methods of screening a candidate anti-TfR binding protein (which may be fused with a therapeutic agent) that may be useful in treating a human disease, and evaluating the efficacy of the candidate human-TfR binding protein fused to a therapeutic agent to reduce, prevent, reduce the likelihood of, and/or inhibit the one or more symptoms of a human disease, wherein the disease is selected from the group consisting of the diseases listed in Table 2.

TABLE 2

| Tissue/Organelle: | Omim Disease Name |
|---|---|
| Lysosomal targets | Tangier disease |
| | Intellectual developmental disorder with poor growth and with or |
| | without seizures or ataxia |
| | Hypermethioninemia due to adenosine kinase deficiency |
| | Aspartylglycosaminuria |
| | Fructose intolerance, hereditary |
| | MEDNIK syndrome |
| | Spastic paraplegia 50, autosomal recessive |
| | Sea-blue histiocyte disease |
| | Adenine phosphoribosyltransferase deficiency |
| | Maroteaux-Lamy syndrome (mucopolysaccharidosis type VI |
| | Mucopolysaccharidosis, type X |
| | Spinocerebellar ataxia, autosomal recessive |
| | Cutis laxa, autosomal recessive, type IID |
| | Farber disease |
| | Hermansky-Pudlak syndrome |
| | Ceroid lipofuscinosis, neuronal, 6A |
| | Ceroid lipofuscinosis, neuronal, 6B (Kufs type) |
| | Ceroid lipofuscinosis, neuronal, 8 |
| | Ceroid lipofuscinosis, neuronal, 8, Northern epilepsy variant |
| | Galactosialidosis |
| | cystinosis |
| | Haim-Munk syndrome, Papillon Lefevre Syndrome |
| | Ceroid lipofuscinosis, neuronal, 10 |
| | Pycnodysostosis |
| | Imerslund-Grasbeck syndrome 1 |
| | Proteinuria, chronic benign] |
| | WHIM syndrome 2 |
| | Orthostatic hypotension 2 |
| | 5-fluorouracil toxicity |
| | Dihydropyrimidine dehydrogenase deficiency |
| | Cone-rod dystrophy 21 |
| | Vici syndrome |
| | Arthrogryposis multiplex congenita 2, neurogenic type |
| | Fucosidosis |
| | Cataract 18, autosomal recessive |
| | Pompe disease |
| | Mucopolysaccharidosis IV |
| | Gaucher disease |
| | Fabry disease |
| | Mucolipidosis II alpha/beta |
| | Mucolipidosis III alpha/beta |
| | Mucolipidosis III gamma |
| | Mucopolysaccharidosis Type VII |
| | Tay Sachs Disease |
| | Sandhoff disease, infantile, juvenile, and adult forms |
| | Mucopolysaccharidosis type IIIC (Sanfilippo C) |
| | Retinitis pigmentosa 73 |
| | Hermansky-Pudlak syndrome 6 |
| | Mucopolysaccharidosis I |
| | Mucopolysaccharidosis II |
| | Spastic paraplegia, optic atrophy, and neuropathy |
| | Lysosomal acid lipase defciency |
| | Danon disease |
| | Immunodeficiency 52 |
| | Leydig cell hypoplasia with hypergonadotropic hypogonadism |
| | Leydig cell hypoplasia with pseudohermaphroditism |
| | Luteinizing hormone resistance, female |
| | Immunodeficiency, common variable, 8, with autoimmunity |
| | Keratosis pilaris atrophicans |
| | Chediak-Higashi syndrome |
| | Alpha-Mannosidosis |
| | Spondyloepiphyseal Dysplasia, Kondo-Fu Type |
| | Mucolipidosis IV |
| | Ceroid lipofuscinosis, neuronal, 7 |
| | Macular dystrophy with central cone involvement |
| | Megalencephalic leukoencephalopathy with subcortical cysts |
| | Myeloperoxidase deficiency |
| | Deafness, autosomal recessive 2 |
| | Usher syndrome, type 1B |
| | Kanzaki disease |
| | Schindler disease, type I |
| | Schindler disease, type III |
| | Niemann-Pick disease, type C1 |
| | Niemann-Pick disease, type D |
| | Niemann-pick disease, type C2 |
| | Spastic paraplegia 45, autosomal recessive |
| | Sialidosis |
| | Parkinson disease 6, early onset |
| | Osteopetrosis, autosomal recessive 6 |
| | Hemophagocytic lymphohistiocytosis, familial, 2 |
| | Epilepsy, progressive myoclonic 4, with or without renal failure |
| | Mucopolysaccharidosis type IIIA (Sanfilippo A) |
| | Neurodevelopmental disorder with cardiomyopathy, spasticity, and |
| | brain abnormalities |
| | Histiocytosis-lymphadenopathy plus syndrome |
| | Niemann-Pick disease, type A/B, acid sphingomyelinase deficiency |
| | Congenital disorder of glycosylation, type IIn |
| | Spinocerebellar ataxia, autosomal recessive 20 |
| | Amyotrophic lateral sclerosis 5, juvenile |
| | Charcot-Marie-Tooth disease, axonal, type 2X |
| | Spastic paraplegia 11, autosomal recessive |
| | Warburg micro syndrome 4 |
| | Dystonia 32 |
| | Leukodystrophy, hypomyelinating, 12 |
| | Choreoacanthocytosis |
| | Arthrogryposis, renal dysfunction, and cholestasis 1 |
| | Pontocerebellar hypoplasia, type 13 |
| | Pontocerebellar hypoplasia, type 2E |
| | Neurodevelopmental disorder with spastic quadriplegia and brain |
| | abnormalities with or without seizures |
| | Cerebellar ataxia, mental retardation, and dysequilibrium syndrome 2 |
| | Hydrocephalus, congenital, 3, with brain anomalies |
| | Xanthinuria, type I |
| | Spastic paraplegia 15, autosomal recessive |
| | Intellectual disability and myopathy syndrome |
| Heart | Muscular dystrophy, limb-girdle, autosomal recessive 25 |
| | Neurodevelopmental disorder with seizures and nonepileptic |
| | hyperkinetic movements |
| | Cerebellar atrophy with seizures and variable developmental delay |
| | Ventricular tachycardia, catecholaminergic polymorphic, 2 |
| | Lipodystrophy, congenital generalized, type 3 |
| | Arrhythmogenic right ventricular dysplasia 11 |
| | Arrhythmogenic right ventricular dysplasia 11 with mild palmoplantar |
| | keratoderma and woolly hair |
| | Cardiomyopathy, dilated, with woolly hair and keratoderma |
| | Epidermolysis bullosa, lethal acantholytic |
| | Skin fragility-woolly hair syndrome |
| | Congenital heart defects, multiple types, 5 |
| | Hemolytic anemia due to glutathione peroxidase deficiency |
| | Naxos disease |
| | Jervell and Lange-Nielsen syndrome 2 |
| | Myopathy, myofibrillar, 12, infantile-onset, with cardiomyopathy |
| | Cardiomyopathy, hypertrophic, 8 |
| | Nephrotic syndrome, type 22 |
| | Developmental and epileptic encephalopathy 52 |
| | Dicarboxylic aminoaciduria |
| | Lichtenstein-Knorr syndrome |
| | Hypogonadotropic hypogonadism 11 with or without anosmia |
| | Segawa syndrome, recessive |
| Central nervous | Intellectual developmental disorder with poor growth and with or |
| system | without seizures or ataxia |
| | Spondyloepimetaphyseal dysplasia, aggrecan type |
| | Neurodevelopmental disorder with hypotonia, microcephaly, and |
| | seizures |
| | Microcephaly 16, primary, autosomal recessive |
| | Spinocerebellar ataxia, autosomal recessive 31 |
| | Acromesomelic dysplasia 3 |
| | Elsahy-Waters syndrome |
| | Ceroid lipofuscinosis, neuronal, 8 |
| | Ceroid lipofuscinosis, neuronal, 8, Northern epilepsy variant |
| | Pitt-Hopkins like syndrome 1 |
| | Gaze palsy, familial horizontal, with progressive scoliosis, 2 |
| | Short-rib thoracic dysplasia 3 with or without polydactyly |
| | Bleeding disorder, platelet-type, 22 |
| | Macrocephaly, dysmorphic facies, and psychomotor retardation |
| | Charcot-Marie-Tooth disease, axonal, type 2S |
| | Neuronopathy, distal hereditary motor, type VI |
| | SESAME syndrome |
| | Goldberg-Shprintzen megacolon syndrome |
| | Obesity, morbid, due to leptin deficiency |
| | Spastic paraplegia 75, autosomal recessive |
| | Hypogonadotropic hypogonadism 27 without anosmia |
| | Seckel syndrome 7 |
| | Pitt-Hopkins-like syndrome 2 |
| | Oxoglutarate dehydrogenase deficiency |
| | Myopathy, congenital, progressive, with scoliosis |
| | Epilepsy, progressive myoclonic, 10 |
| | Lissencephaly 2 (Norman-Roberts type) |
| | Thyroid hormone metabolism, abnormal |
| | Thyroid hormone metabolism, abnormal, 1 |
| | Neuropathy, hereditary motor and sensory, type VIB |
| | Pontocerebellar hypoplasia, type 1E |
| | Amyotrophic lateral sclerosis 5, juvenile |
| | Charcot-Marie-Tooth disease, axonal, type 2X |
| | Spastic paraplegia 11, autosomal recessive |
| | Netherton syndrome |
| | Joubert syndrome 13 |
| | Microphthalmia, syndromic 11 |
| | Osteogenesis imperfecta, type XV |
| Eye | Intellectual developmental disorder with poor growth and with or |
| | without seizures or ataxia |
| | Microcornea, myopic chorioretinal atrophy, and telecanthus |
| | Microphthalmia, isolated 8 |
| | Fructose intolerance, hereditary |
| | Alstrom syndrome |
| | Sea-blue histiocyte disease |
| | Mucopolysaccharidosis, type X |
| | Cutis laxa, autosomal recessive, type IID |
| | Bardet-Biedl syndrome 4 |
| | Bardet-Biedl syndrome 7 |
| | Acromesomelic dysplasia 3 |
| | Cone-rod synaptic disorder, congenital nonprogressive |
| | Joubert syndrome 5 |
| | Leber congenital amaurosis 10 |
| | Meckel syndrome 4 |
| | Senior-Loken syndrome 6 |
| | Complement factor D deficiency |
| | Ceroid lipofuscinosis, neuronal, 8 |
| | Ceroid lipofuscinosis, neuronal, 8, Northern epilepsy variant |
| | Achromatopsia 2 |
| | Focal segmental glomerulosclerosis 9 |
| | Ventriculomegaly with cystic kidney disease |
| | Leber congenital amaurosis 7 |
| | Cataract 22 |
| | Galactosialidosis |
| | Ceroid lipofuscinosis, neuronal, 10 |
| | Pycnodysostosis |
| | Imerslund-Grasbeck syndrome 1 |
| | Proteinuria, chronic benign] |
| | WHIM syndrome 2 |
| | Cone-rod dystrophy 21 |
| | Vici syndrome |
| | Bleeding disorder, platelet-type, 22 |
| | Anterior segment dysgenesis 2, multiple subtypes |
| | Fucosidosis |
| | Cataract 18, autosomal recessive |
| | Ectodermal dysplasia/short stature syndrome |
| | Night blindness, congenital stationary (complete), 1B, autosomal |
| | recessive |
| | Growth hormone deficiency with pituitary anomalies |
| | Pituitary hormone deficiency, combined, 5 |
| | Septooptic dysplasia |
| | Sandhoff disease, infantile, juvenile, and adult forms |
| | Mucopolysaccharidosis type IIIC (Sanfilippo C) |
| | Retinitis pigmentosa 73 |
| | Hermansky-Pudlak syndrome 6 |
| | Cerebellar atrophy, developmental delay, and seizures |
| | Cornea plana 2, autosomal recessive |
| | Spastic paraplegia, optic atrophy, and neuropathy |
| | Poretti-Boltshauser syndrome |
| | Cortical malformations, occipital |
| | Leydig cell hypoplasia with hypergonadotropic hypogonadism |
| | Leydig cell hypoplasia with pseudohermaphroditism |
| | Luteinizing hormone resistance, female |
| | Immunodeficiency, common variable, 8, with autoimmunity |
| | Microphthalmia/coloboma and skeletal dysplasia syndrome |
| | Charcot-Marie-Tooth disease, axonal, type 2A2B |
| | Neurodevelopmental disorder with progressive microcephaly, |
| | spasticity, and brain abnormalities |
| | Ceroid lipofuscinosis, neuronal, 7 |
| | Macular dystrophy with central cone involvement |
| | Megalencephalic leukoencephalopathy with subcortical cysts |
| | Myeloperoxidase deficiency |
| | Kanzaki disease |
| | Schindler disease, type I |
| | Schindler disease, type III |
| | Niemann-Pick disease, type C1 |
| | Niemann-Pick disease, type D |
| | Niemann-pick disease, type C2 |
| | Joubert syndrome 4 |
| | Nephronophthisis 1, juvenile |
| | Senior-Loken syndrome-1 |
| | Microcephalic osteodysplastic primordial dwarfism, type II |
| | Retinitis pigmentosa 43 |
| | Retinitis pigmentosa-40 |
| | Cataract 11, multiple types |
| | Cataract 11, syndromic, autosomal recessive |
| | Osteopetrosis, autosomal recessive 6 |
| | Hemophagocytic lymphohistiocytosis, familial, 2 |
| | Microphthalmia, isolated 6 |
| | Anterior segment dysgenesis 7, with sclerocornea |
| | Martsolf syndrome 2 |
| | Warburg micro syndrome 1 |
| | Retinal dystrophy, iris coloboma, and comedogenic acne syndrome |
| | Leber congenital amaurosis 12 |
| | COACH syndrome 3 |
| | Joubert syndrome 7 |
| | Meckel syndrome 5 |
| | Intellectual developmental disorder and retinitis pigmentosa |
| | Epilepsy, progressive myoclonic 4, with or without renal failure |
| | Mucopolysaccharidosis type IIIA (Sanfilippo A) |
| | Dicarboxylic aminoaciduria |
| | Histiocytosis-lymphadenopathy plus syndrome |
| | Congenital disorder of glycosylation, type IIn |
| | Heart and brain malformation syndrome |
| | Microphthalmia with limb anomalies |
| | Spinocerebellar ataxia, autosomal recessive 20 |
| | Deafness, autosomal recessive 115 |
| | Warburg micro syndrome 4 |
| | Segawa syndrome, recessive |
| | Night blindness, congenital stationary (complete), 1C, autosomal |
| | recessive |
| | Focal facial dermal dysplasia 3, Setleis type |
| | Deafness, autosomal recessive 18A |
| | Usher syndrome, type 1C |
| | Microphthalmia, syndromic 11 |
| | Dystonia 32 |
| | Leukodystrophy, hypomyelinating, 12 |
| | Choreoacanthocytosis |
| | Arthrogryposis, renal dysfunction, and cholestasis 1 |
| | Neurodevelopmental disorder with spastic quadriplegia and brain |
| | abnormalities with or without seizures |
| | Cerebellar ataxia, mental retardation, and dysequilibrium syndrome 2 |
| | Hydrocephalus, congenital, 3, with brain anomalies |
| | Spastic paraplegia 15, autosomal recessive |
| Brain | Deafness, autosomal recessive 44 |
| | Microcephaly 5, primary, autosomal recessive |
| | Spinocerebellar ataxia, autosomal recessive 31 |
| | Bardet-Biedl syndrome 2 |
| | Retinitis pigmentosa 74 |
| | Bardet-Biedl syndrome 4 |
| | Pitt-Hopkins like syndrome 1 |
| | Joubert syndrome 17 |
| | Orofaciodigital syndrome VI |
| | Oculocutaneous albinism, type VIII |
| | Hermansky-Pudlak syndrome 7 |
| | Short-rib thoracic dysplasia 3 with or without polydactyly |
| | Macrocephaly, dysmorphic facies, and psychomotor retardation |
| | Growth hormone deficiency with pituitary anomalies |
| | Pituitary hormone deficiency, combined, 5 |
| | Septooptic dysplasia |
| | Intellectual developmental disorder, autosomal recessive 57 |
| | Neurodevelopmental disorder with progressive microcephaly, |
| | spasticity, and brain abnormalities |
| | Hypogonadotropic hypogonadism 27 without anosmia |
| | Pitt-Hopkins-like syndrome 2 |
| | Oxoglutarate dehydrogenase deficiency |
| | Microcephalic osteodysplastic primordial dwarfism, type II |
| | Intellectual developmental disorder with paroxysmal dyskinesia or |
| | seizures |
| | Neurodevelopmental disorder with dysmorphic features, spasticity, and |
| | brain abnormalities |
| | Developmental and epileptic encephalopathy 12 |
| | Martsolf syndrome 2 |
| | Warburg micro syndrome 1 |
| | Lissencephaly 2 (Norman-Roberts type) |
| | COACH syndrome 3 |
| | Joubert syndrome 7 |
| | Meckel syndrome 5 |
| | Thyroid hormone metabolism, abnormal |
| | Thyroid hormone metabolism, abnormal, 1 |
| | Neuropathy, hereditary motor and sensory, type VIB |
| | Pontocerebellar hypoplasia, type 1E |
| | Spinocerebellar ataxia, autosomal recessive 14 |
| | Microcephaly-capillary malformation syndrome |
| | Neurodevelopmental disorder, nonprogressive, with spasticity and |
| | transient opisthotonus |
| | Intellectual developmental disorder, autosomal recessive 13 |
| | Microcephaly 2, primary, autosomal recessive, with or without cortical |
| | malformations |
| | Osteogenesis imperfecta, type XV |
| | Diarrhea 9 |
| Spinal cord | Neurodevelopmental disorder with hypotonia, microcephaly, and |
| | seizures |
| | Ceroid lipofuscinosis, neuronal, 8 |
| | Ceroid lipofuscinosis, neuronal, 8, Northern epilepsy variant |
| | Nephrotic syndrome, type 24 |
| | Gaze palsy, familial horizontal, with progressive scoliosis, 2 |
| | Short-rib thoracic dysplasia 3 with or without polydactyly |
| | Charcot-Marie-Tooth disease, axonal, type 2S |
| | Neuronopathy, distal hereditary motor, type VI |
| | Myopathy, congenital, progressive, with scoliosis |
| | Neu-Laxova syndrome 1 |
| | Phosphoglycerate dehydrogenase deficiency |
| | Carpenter syndrome |
| | Lissencephaly 2 (Norman-Roberts type) |
| | Joubert syndrome 13 |
| | Cerebellar hypoplasia and mental retardation with or without |
| | quadrupedal locomotion 1 |
| | Osteogenesis imperfecta, type XV |
| | OMIM_disease_name |
| Peripheral | Visceral neuropathy, familial, 2, autosomal recessive |
| nervous system | |
| | Arthrogryposis multiplex congenita 1, neurogenic, with myelin defect |
| | Multicentric osteolysis, nodulosis, and arthropathy |
| | Charcot-Marie-Tooth disease, type 4D |
| | Hypogonadotropic hypogonadism 27 without anosmia |
| | Charcot-Marie-Tooth disease, type 4C |
| | Neuropathy, hereditary motor and sensory, type VIB |
| | Pontocerebellar hypoplasia, type 1E |
| | Encephalopathy, progressive, with amyotrophy and optic atrophy |
| | Hypoparathyroidism-retardation-dysmorphism syndrome |
| | Kenny-Caffey syndrome, type 1 |
| Skeletal muscle | Brody myopathy |
| | Muscular dystrophy, limb-girdle, autosomal recessive 25 |
| | Lipodystrophy, congenital generalized, type 3 |
| | Myasthenic syndrome, congenital, 1B, fast-channel |
| | Myasthenic syndrome, congenital, 3B, fast-channel |
| | Myasthenic syndrome, congenital, 3C, associated with acetylcholine |
| | receptor deficiency |
| | Ceroid lipofuscinosis, neuronal, 8 |
| | Ceroid lipofuscinosis, neuronal, 8, Northern epilepsy variant |
| | Spondylocarpotarsal synostosis syndrome |
| | Hemolytic anemia due to glutathione peroxidase deficiency |
| | Gillespie syndrome |
| | Nemaline myopathy 10 |
| | Myopathy, congenital, progressive, with scoliosis |
| | Myasthenic syndrome, congenital, 16 |
| | Dystonia, dopa-responsive, due to sepiapterin reductase deficiency |
| | Split-hand/foot malformation 6 |
| Cartilage | Spondyloepimetaphyseal dysplasia, aggrecan type |
| | Bardet-Biedl syndrome 2 |
| | Retinitis pigmentosa 74 |
| | Acromesomelic dysplasia 3 |
| | Osteochondrodysplasia, brachydactyly, and overlapping malformed |
| | digits |
| | Temtamy preaxial brachydactyly syndrome |
| | Fibrochondrogenesis 1 |
| | Deafness, autosomal recessive 53 |
| | Fibrochondrogenesis 2 |
| | Otospondylomegaepiphyseal dysplasia, autosomal recessive |
| | Steel syndrome |
| | Pycnodysostosis |
| | Spondyloepimetaphyseal dysplasia, Shohat type |
| | Acromesomelic dysplasia 2A |
| | Acromesomelic dysplasia 2B |
| | Acromesomelic dysplasia 2C, Hunter-Thompson type |
| | Brachydactyly, type A1, C |
| | Leber congenital amaurosis 17 |
| | Short-rib thoracic dysplasia 2 with or without polydactyly |
| | Obesity, morbid, due to leptin deficiency |
| | Neurodevelopmental disorder with epilepsy and hypoplasia of the |
| | corpus callosum |
| | Myopathy, congenital, progressive, with scoliosis |
| | Rhizomelic limb shortening with dysmorphic features |
| | Short stature, onychodysplasia, facial dysmorphism, and hypotrichosis |
| | Hypoparathyroidism, familial isolated 1 |
| | Robinow syndrome, autosomal recessive |
| | Spondyloepimetaphyseal dysplasia, Krakow type |
| | Congenital disorder of glycosylation, type IIn |
| | Waardenburg syndrome, type 2D |
| Bone growth | Ehlers-Danlos syndrome, cardiac valvular type |
| plate | |
| | Steel syndrome |
| | Factor VII deficiency |
| | Short-rib thoracic dysplasia 2 with or without polydactyly |
| | Keratosis pilaris atrophicans |
| | Short stature, onychodysplasia, facial dysmorphism, and hypotrichosis |
| | Nephrotic syndrome, type 14 |
| | Waardenburg syndrome, type 2D |
| | OMIM_disease_name |
| Kidney | Cone-rod dystrophy 3 |
| | Fundus flavimaculatus |
| | Retinal dystrophy, early-onset severe |
| | Retinitis pigmentosa 19 |
| | Stargardt disease 1 |
| | Lethal congenital contracture syndrome 8 |
| | Nephronophthisis 16 |
| | Distal renal tubular acidosis 3, with or without sensorineural hearing |
| | loss |
| | Distal renal tubular acidosis 2 with progressive sensorineural hearing |
| | loss |
| | Bardet-Biedl syndrome 2 |
| | Retinitis pigmentosa 74 |
| | Deafness, autosomal recessive 93 |
| | Cone-rod synaptic disorder, congenital nonprogressive |
| | Joubert syndrome 5 |
| | Leber congenital amaurosis 10 |
| | Meckel syndrome 4 |
| | Senior-Loken syndrome 6 |
| | Bartter syndrome, type 3 |
| | Deafness, autosomal recessive 103 |
| | Ceroid lipofuscinosis, neuronal, 6A |
| | Ceroid lipofuscinosis, neuronal, 6B (Kufs type) |
| | Ceroid lipofuscinosis, neuronal, 8 |
| | Ceroid lipofuscinosis, neuronal, 8, Northern epilepsy variant |
| | Retinitis pigmentosa 61 |
| | Usher syndrome, type 3A |
| | Achromatopsia 2 |
| | Fibrochondrogenesis 1 |
| | Joubert syndrome 17 |
| | Orofaciodigital syndrome VI |
| | Leber congenital amaurosis 7 |
| | Cataract 22 |
| | Chronic granulomatous disease 4, autosomal recessive |
| | Cone-rod dystrophy 21 |
| | Short-rib thoracic dysplasia 3 with or without polydactyly |
| | Leber congenital amaurosis 17 |
| | Hyperekplexia 2 |
| | Night blindness, congenital stationary (complete), 1B, autosomal |
| | recessive |
| | Immunodeficiency-centromeric instability-facial anomalies syndrome |
| | 4 |
| | Muscular dystrophy, congenital, with cataracts and intellectual |
| | disability |
| | Renal hypodysplasia/aplasia 1 |
| | SESAME syndrome |
| | Cerebellar atrophy, developmental delay, and seizures |
| | Pseudohypoaldosteronism, type IID |
| | Cortical malformations, occipital |
| | Leber congenital amaurosis 14 |
| | Retinal dystrophy, early-onset severe |
| | Retinitis pigmentosa, juvenile |
| | Night blindness, congenital stationary (complete), 1F, autosomal |
| | recessive |
| | Metaphyseal anadysplasia 2 |
| | Deafness, autosomal recessive 30 |
| | Deafness, autosomal recessive 2 |
| | Usher syndrome, type 1B |
| | Short-rib thoracic dysplasia 6 with or without polydactyly |
| | Meckel syndrome 7 |
| | Nephronophthisis 3 |
| | Renal-hepatic-pancreatic dysplasia 1 |
| | Boudin-Mortier syndrome |
| | Microcephalic osteodysplastic primordial dwarfism, type II |
| | Retinitis pigmentosa 43 |
| | Retinitis pigmentosa-40 |
| | Leber congenital amaurosis 12 |
| | Bothnia retinal dystrophy |
| | Newfoundland rod-cone dystrophy |
| | COACH syndrome 3 |
| | Joubert syndrome 7 |
| | Meckel syndrome 5 |
| | Nephrotic syndrome, type 14 |
| | Leber congenital amaurosis 3 |
| | Retinitis pigmentosa 94, variable age at onset, autosomal recessive |
| | Immunodeficiency 31B, mycobacterial and viral infections, autosomal |
| | recessive |
| | Corneal dystrophy, gelatinous drop-like |
| | Segawa syndrome, recessive |
| | COACH syndrome 1 |
| | Joubert syndrome 6 |
| | Meckel syndrome 3 |
| | Nephronophthisis 11 |
| | RHYNS syndrome |
| | Night blindness, congenital stationary (complete), 1C, autosomal |
| | recessive |
| | Diarrhea 9 |
| | Nephronophthisis-like nephropathy 1 |
| Blood | Combined oxidative phosphorylation deficiency 8 |
| | Leukoencephalopathy, progressive, with ovarian failure |
| | 2-methylbutyrylglycinuria |
| | Alpha-methylacetoacetic aciduria |
| | Aicardi-Goutieres syndrome 6 |
| | Neurodevelopmental disorder with hypotonia, microcephaly, and |
| | seizures |
| | Deafness, autosomal recessive 44 |
| | Obesity, susceptibility to, BMIQ19} |
| | Lethal congenital contracture syndrome 8 |
| | Hypermethioninemia due to adenosine kinase deficiency |
| | Neurodegeneration, childhood-onset, stress-induced, with variable |
| | ataxia and seizures |
| | Alopecia-intellectual disability syndrome 1 |
| | Immunodeficiency with hyper-IgM, type 2 |
| | Leukodystrophy, hypomyelinating, 3 |
| | Autoimmune polyendocrinopathy syndrome, type I, with or without |
| | reversible metaphyseal dysplasia |
| | Spermatogenic failure 27 |
| | Glycogen storage disease XII |
| | Fructose intolerance, hereditary |
| | Intellectual developmental disorder, autosomal recessive 71 |
| | Myopathy due to myoadenylate deaminase deficiency |
| | Pontocerebellar hypoplasia, type 9 |
| | Spastic paraplegia 63 |
| | Ferguson-Bonni neurodevelopmental syndrome |
| | Scott syndrome |
| | Spastic paraplegia 48, autosomal recessive |
| | Adenine phosphoribosyltransferase deficiency |
| | Ataxia, early-onset, with oculomotor apraxia and hypoalbuminemia |
| | Spinal muscular atrophy with congenital bone fractures 2 |
| | Cutis laxa, autosomal recessive, type IID |
| | Distal renal tubular acidosis 2 with progressive sensorineural hearing |
| | loss |
| | Muscular dystrophy-dystroglycanopathy (congenital with brain and |
| | eye anomalies, type A, 11 |
| | Bile acid conjugation defect 1 |
| | Agammaglobulinemia 4 |
| | Hermansky-Pudlak syndrome 9 |
| | Acromesomelic dysplasia 3 |
| | Erythrocytosis, familial, 8 |
| | Fanconi anemia, complementation group J |
| | Desbuquois dysplasia 1 |
| | Epiphyseal dysplasia, multiple, 7 |
| | Immunodeficiency 11A |
| | Immunodeficiency, common variable, 3 |
| | Lymphoproliferative syndrome 2 |
| | Immunodeficiency with hyper-IgM, type 3 |
| | Deafness, autosomal recessive 32, with or without immotile sperm |
| | Microcephaly 12, primary, autosomal recessive |
| | Microcephaly 13, primary, autosomal recessive |
| | Nephronophthisis 15 |
| | Complement factor B deficiency |
| | Cocoon syndrome |
| | Popliteal pterygium syndrome, Bartsocas-Papas type 2 |
| | Cold-induced sweating syndrome 2 |
| | Leukodystrophy, hypomyelinating, 20 |
| | Pitt-Hopkins like syndrome 1 |
| | Neurodegeneration with brain iron accumulation 6 |
| | Pontocerebellar hypoplasia, type 12 |
| | Carbamoylphosphate synthetase I deficiency |
| | Surfactant metabolism dysfunction, pulmonary, 5 |
| | Neutropenia, severe congenital, 7, autosomal recessive |
| | Joubert syndrome 21 |
| | Cerebroretinal microangiopathy with calcifications and cysts |
| | Microcephaly, facial dysmorphism, renal agenesis, and ambiguous |
| | genitalia syndrome |
| | WHIM syndrome 2 |
| | Aromatase deficiency |
| | Bile acid synthesis defect, congenital, 3 |
| | Spastic paraplegia 5A, autosomal recessive |
| | Developmental and epileptic encephalopathy 86 |
| | Leukoencephalopathy with brain stem and spinal cord involvement and |
| | lactate elevation |
| | Oculocutaneous albinism, type VIII |
| | Pentosuria] |
| | Aromatic L-amino acid decarboxylase deficiency |
| | Mitochondrial DNA depletion syndrome 3 (hepatocerebral type) |
| | Progressive external ophthalmoplegia with mitochondrial DNA |
| | deletions, autosomal recessive 4 |
| | Miller syndrome |
| | Pyruvate dehydrogenase E2 deficiency |
| | Systemic lupus erythematosus 16 |
| | Immunodeficiency-centromeric instability-facial anomalies |
| | syndrome 1 |
| | Immunodeficiency 40 |
| | Congenital disorder of glycosylation, type Ie |
| | 5-fluorouracil toxicity |
| | Dihydropyrimidine dehydrogenase deficiency |
| | Intellectual developmental disorder, autosomal recessive 50 |
| | Combined oxidative phosphorylation deficiency 17 |
| | Dysautonomia, familial |
| | Spastic paraplegia 64, autosomal recessive |
| | Bleeding disorder, platelet-type, 22 |
| | Eosinophil peroxidase deficiency] |
| | Visceral neuropathy, familial, 2, autosomal recessive |
| | Fanconi anemia, complementation group Q |
| | XFE progeroid syndrome |
| | Xeroderma pigmentosum, group F |
| | Xeroderma pigmentosum, type F/Cockayne syndrome |
| | Cerebrooculofacioskeletal syndrome 3 |
| | Xeroderma pigmentosum, group G |
| | Xeroderma pigmentosum, group G/Cockayne syndrome |
| | Cockayne syndrome, type A |
| | UV-sensitive syndrome 2 |
| | Deafness, autosomal recessive 109 |
| | Pontocerebellar hypoplasia, type 1F |
| | Dysprothrombinemia |
| | Hypoprothrombinemia |
| | Immunodeficiency 90 with encephalopathy, functional hyposplenia, |
| | and hepatic dysfunction |
| | Raine syndrome |
| | Fanconi anemia, complementation group D2 |
| | Fanconi anemia, complementation group I |
| | Fanconi anemia, complementation group L |
| | Peroxisomal fatty acyl-CoA reductase 1 disorder |
| | Combined oxidative phosphorylation deficiency 14 |
| | Spastic paraplegia 77, autosomal recessive |
| | Rajab interstitial lung disease with brain calcifications 2 |
| | Combined oxidative phosphorylation deficiency 44 |
| | Parkinson disease 15, autosomal recessive |
| | Leukocyte adhesion deficiency, type III |
| | Siddiqi syndrome |
| | Anterior segment dysgenesis 2, multiple subtypes |
| | T-cell immunodeficiency, congenital alopecia, and nail dystrophy |
| | Combined oxidative phosphorylation deficiency 41 |
| | Glutaricaciduria, type I |
| | Diabetes mellitus, permanent neonatal 1 |
| | Bleeding disorder, platelet-type, 17 |
| | Nonaka myopathy |
| | Hypertriglyceridemia, transient infantile |
| | Chudley-McCullough syndrome |
| | Jaberi-Elahi syndrome |
| | Combined oxidative phosphorylation deficiency 23 |
| | Vertebral, cardiac, renal, and limb defects syndrome 1 |
| | T-cell lymphoma, subcutaneous panniculitis-like |
| | Immunodeficiency-centromeric instability-facial anomalies |
| | syndrome 4 |
| | Hemochromatosis, type 2A |
| | Heme oxygenase-1 deficiency |
| | Dystonia 2, torsion, autosomal recessive |
| | D-bifunctional protein deficiency |
| | Perrault syndrome 1 |
| | Premature ovarian failure 19 |
| | Immunodeficiency 27A, mycobacteriosis, AR |
| | Charcot-Marie-Tooth disease, axonal, type 2S |
| | Neuronopathy, distal hereditary motor, type VI |
| | Immunodeficiency 15B |
| | Immunodeficiency 29, mycobacteriosis |
| | Immunodeficiency 30 |
| | Candidiasis, familial, 9 |
| | Immunodeficiency, common variable, 11 |
| | Immunodeficiency 56 |
| | Immunodeficiency 41 with lymphoproliferation and autoimmunity |
| | Immunodeficiency 63 with lymphoproliferation and autoimmunity |
| | Immunodeficiency 39 |
| | Immunodeficiency 32B, monocyte and dendritic cell deficiency, |
| | autosomal recessive |
| | Autoimmune disease, multisystem, with facial dysmorphism |
| | Lymphoproliferative syndrome 1 |
| | Muscular dystrophy, limb-girdle, autosomal recessive 27 |
| | SCID, autosomal recessive, T-negative/B-positive type |
| | Basal ganglia calcification, idiopathic, 8, autosomal recessive |
| | Hemorrhagic destruction of the brain, subependymal calcification, and |
| | cataracts |
| | Hydroxykynureninuria |
| | Vertebral, cardiac, renal, and limb defects syndrome 2 |
| | Immunodeficiency 52 |
| | Immunodeficiency 81 |
| | Obesity, morbid, due to leptin deficiency |
| | Obesity, morbid, due to leptin receptor deficiency |
| | Lipodystrophy, familial partial, type 6 |
| | Chediak-Higashi syndrome |
| | 3-Methylcrotonyl-CoA carboxylase 2 deficiency |
| | Basel-Vanagait-Smirin-Yosef syndrome |
| | Intellectual developmental disorder, autosomal recessive 72 |
| | Mitochondrial DNA depletion syndrome 11 |
| | Mismatch repair cancer syndrome 1 |
| | Metaphyseal anadysplasia 2 |
| | Xanthinuria, type II |
| | Molybdenum cofactor deficiency B |
| | Thrombocytopenia, anemia, and myelofibrosis |
| | Deafness, autosomal recessive 111 |
| | Familial adenomatous polyposis 4 |
| | Premature ovarian failure 13 |
| | Vertebral, cardiac, renal, and limb defects syndrome 3 |
| | Encephalopathy, progressive, early-onset, with brain edema and/or |
| | leukoencephalopathy |
| | Infantile liver failure syndrome 2 |
| | Short stature, optic nerve atrophy, and Pelger-Huet anomaly |
| | Microcephaly 22, primary, autosomal recessive |
| | Chronic granulomatous disease 1, autosomal recessive |
| | Chronic granulomatous disease 2, autosomal recessive |
| | Charcot-Marie-Tooth disease, type 4D |
| | Mitochondrial complex I deficiency, nuclear type 22 |
| | Mitochondrial complex I deficiency, nuclear type 37 |
| | Mitochondrial complex I deficiency, nuclear type 32 |
| | Mitochondrial complex I deficiency, nuclear type 24 |
| | Mitochondrial complex I deficiency, nuclear type 5 |
| | Dyskeratosis congenita, autosomal recessive 2 |
| | Glucocorticoid deficiency 4, with or without mineralocorticoid |
| | deficiency |
| | Acromesomelic dysplasia 1, Maroteaux type |
| | Boudin-Mortier syndrome |
| | Spastic paraplegia 45, autosomal recessive |
| | Insensitivity to pain, congenital, with anhidrosis |
| | Striatonigral degeneration, infantile |
| | Nephrotic syndrome, type 17 |
| | Oxoglutarate dehydrogenase deficiency |
| | Hyperphenylalaninemia, non-PKU mild] |
| | Phenylketonuria |
| | Parkinson disease 7, autosomal recessive early-onset |
| | Myopathy, congenital, progressive, with scoliosis |
| | Intellectual developmental disorder with paroxysmal dyskinesia or |
| | seizures |
| | Lacticacidemia due to PDX1 deficiency |
| | Pancreatic agenesis 1 |
| | Neuropathy, hereditary motor and sensory, type VIC, with optic |
| | atrophy |
| | Glycogen storage disease VII |
| | Immunodeficiency 23 |
| | Rhizomelic limb shortening with dysmorphic features |
| | Developmental and epileptic encephalopathy 12 |
| | Osteopetrosis, autosomal recessive 6 |
| | Short stature, onychodysplasia, facial dysmorphism, and hypotrichosis |
| | Mitochondrial DNA depletion syndrome 16 (hepatic type) |
| | Mitochondrial DNA depletion syndrome 16B (neuroophthalmic type) |
| | Hemophagocytic lymphohistiocytosis, familial, 2 |
| | Immunodeficiency 26, with or without neurologic abnormalities |
| | Dystonia 16 |
| | Hypoparathyroidism, familial isolated 1 |
| | Hyperphenylalaninemia, BH4-deficient, A |
| | Myopathy, lactic acidosis, and sideroblastic anemia 1 |
| | Intellectual developmental disorder with abnormal behavior, |
| | microcephaly, and short stature |
| | Leukodystrophy, hypomyelinating, 10 |
| | Combined oxidative phosphorylation deficiency 40 |
| | Carpenter syndrome |
| | Immunodeficiency 73C with defective neutrophil chemotaxis and |
| | hypogammaglobulinemia |
| | Leber congenital amaurosis 12 |
| | Deafness, autosomal recessive 24 |
| | Aicardi-Goutieres syndrome 3 |
| | RIDDLE syndrome |
| | Robinow syndrome, autosomal recessive |
| | Ribose 5-phosphate isomerase deficiency |
| | Mitochondrial complex II deficiency, nuclear type 4 |
| | Spinocerebellar ataxia, autosomal recessive, with axonal neuropathy 2 |
| | Neurodevelopmental disorder with cardiomyopathy, spasticity, and |
| | brain abnormalities |
| | Albinism, oculocutaneous, type VI |
| | Skin/hair/eye pigmentation 4, fair/dark skin] |
| | Citrullinemia, adult-onset type II |
| | Citrullinemia, type II, neonatal-onset |
| | Congenital disorder of glycosylation, type IIn |
| | Parkinsonism-dystonia, infantile, 1 |
| | Lichtenstein-Knorr syndrome |
| | Heart and brain malformation syndrome |
| | Dentin dysplasia, type I, with microdontia and misshapen teeth |
| | Osteopetrosis, autosomal recessive 8 |
| | Ovarian dysgenesis 9 |
| | Deafness, autosomal recessive 115 |
| | Dystonia, dopa-responsive, due to sepiapterin reductase deficiency |
| | Pyropoikilocytosis |
| | Spherocytosis, type 3 |
| | Immunodeficiency 31B, mycobacterial and viral infections, autosomal |
| | recessive |
| | Hemophagocytic lymphohistiocytosis, familial, 4 |
| | Intellectual developmental disorder, autosomal recessive 40 |
| | Hypertryptophanemia |
| | Spinocerebellar ataxia, autosomal recessive, with axonal neuropathy 1 |
| | Osteogenesis imperfecta, type XVIII |
| | Catel-Manzke syndrome |
| | Segawa syndrome, recessive |
| | Spinocerebellar ataxia, autosomal recessive 28 |
| | Paget disease of bone 5, juvenile-onset |
| | Mosaic variegated aneuploidy syndrome 3 |
| | Oocyte maturation defect 9 |
| | Intellectual developmental disorder, autosomal recessive 68 |
| | Immunodeficiency 35 |
| | Beta-ureidopropionase deficiency |
| | Leber congenital amaurosis 19 |
| | Combined oxidative phosphorylation deficiency 20 |
| | Galloway-Mowat syndrome 6 |
| | Microcephaly, growth deficiency, seizures, and brain malformations |
| | Osteogenesis imperfecta, type XV |
| | Split-hand/foot malformation 6 |
| | Dyskeratosis congenita, autosomal recessive 3 |
| | Xanthinuria, type I |
| | Spastic paraplegia 15, autosomal recessive |

CNS disorders and disorders with neurological symptoms amenable to protein therapies (e.g., fusion of an anti-human-TfR binding protein and a therapeutic protein) include, but are not limited to: Alzheimer's, brain cancer, Behcet's Disease, cerebral Lupus, Creutzfeldt-Jakob Disease, dementia, epilepsy, encephalitis, Friedreich's Ataxia, Guillain-Barre Syndrome, Gaucher Disease, headache, hydrocephalus, Huntington's disease, intracranial hypertension, leukodystrophy, migraine, myasthenia gravis, muscular dystrophy, multiple sclerosis, narcolepsy, neuropathy, Prader-Willi Syndrome, Parkinson's disease, Rett Syndrome, restless leg syndrome, sleep disorders, subarachnoid haemorrhage, stroke, traumatic brain injury, trigeminal neuralgia, transient ischaemic attack, and Von Hippel-Lindau Syndrome (angiomatosis).

In some embodiments, a non-human animal as described herein, in addition to expressing a heterologous (e.g., human) TfR protein, exhibits one or more symptoms of an enzyme-deficiency disease and/or a disease selected from the group consisting of Fabry disease, Gaucher disease, MPS I, MPS II, MPS IIIA, MPS IIIB, MPS IIID, MPS IVB, MPS VI, MPS VII, MPS IX, Pompe disease, Lysosomal acid lipase deficiency, Metachromatic leukodystrophy, Niemann-Pick diseases types A, B, and C2, Alpha mannosidosis, Neuraminidase deficiency, Sialidosis, Aspartylglycosaminuria, Combined saposin deficiency, Atypical Gaucher disease, Farber lipogranulomatosis, Fucosidosis, and Beta mannosidosis.

Enzyme-deficiency diseases may include, for example, non-lysosomal storage diseases such as Krabbe disease (galactosylceramidase), phenylketonuria, galactosemia, maple syrup urine disease, mitochondrial disorders, Friedreich ataxia, Zellweger syndrome, adrenoleukodystrophy, Wilson disease, hemochromatosis, ornithine transcarbamylase deficiency, methylmalonic academia, propionic academia, and lysosomal storage diseases. “Lysosomal storage diseases” include any disorder resulting from a defect in lysosome function. Currently, approximately 50 lysosomal storage disorders have been identified, the most well-known of which include Tay-Sachs, Gaucher, and Niemann-Pick disease. The pathogeneses of the diseases are ascribed to the buildup of incomplete degradation products in the lysosome, usually due to loss of protein function. Lysosomal storage diseases are caused by loss-of-function or attenuating variants in the proteins whose normal function is to degrade or coordinate degradation of lysosomal contents. The proteins affiliated with lysosomal storage diseases include enzymes, receptors and other transmembrane proteins (e.g., NPC1), post-translational modifying proteins (e.g., sulfatase), membrane transport proteins, and non-enzymatic cofactors and other soluble proteins (e.g., GM2 ganglioside activator). Thus, lysosomal storage diseases encompass more than those disorders caused by defective enzymes per se, and include any disorder caused by any molecular defect. Thus, as used herein, the term “enzyme” is meant to encompass those other proteins associated with lysosomal storage diseases.

The nature of the molecular lesion affects the severity of the disease in many cases, i.e. complete loss-of-function tends to be associated with pre-natal or neo-natal onset, and involves severe symptoms; partial loss-of-function is associated with milder (relatively) and later-onset disease. Generally, only a small percentage of activity needs to be restored to have to correct metabolic defects in deficient cells.

Lysosomal storage diseases are a class of rare diseases that affect the degradation of myriad substrates in the lysosome. Those substrates include sphingolipids, mucopolysaccharides, glycoproteins, glycogen, and oligosaccharides, which can accumulate in the cells of those with disease leading to cell death. Organs affected by lysosomal storage diseases include the central nervous system (CNS), the peripheral nervous system (PNS), lungs, liver, bone, skeletal and cardiac muscle, and the reticuloendothelial system.

Options for the treatment of lysosomal storage diseases include enzyme replacement therapy (ERT), substrate reduction therapy, pharmacological chaperone-mediated therapy, hematopoietic stem cell transplant therapy, and gene therapy. An example of substrate reduction therapy includes the use of Miglustat or Eliglustat to treat Gaucher Type 1. These drugs act by blocking synthase activity, which reduces subsequent substrate production. Hematopoietic stem cell therapy (HSCT), for example, is used to ameliorate and slow-down the negative central nervous system phenotype in patients with some forms of MPS. See R. M. Boustany, “Lysosomal storage diseases—the horizon expands,” 9(10) Nat. Rev. Neurol. 583-98, October 2013; which reference is incorporated herein in its entirety by reference.

Two of the most common LSDs are Pompe disease and Fabry disease. Pompe disease, which has an estimated incidence of 1 in 10,000, is caused by defective lysosomal enzyme alpha-glucosidase (GAA). GAA hydrolyzes terminal non-reducing (1→4)-linked alpha-glucose residues to release a single alpha-glucose molecule. GAA is a carbohydrate-hydrolase that releases α-glucose, not β-glucose, because of the affinities of GAA's active site. GAA is encoded by the Gaa gene, and non-limiting examples of the amino acid and nucleic acid molecules of a mouse GAA enzyme and a mouse Gaa gene are set forth as SEQ ID NO:48 and SEQ ID NO:49, respectively.

Dysfunction in GAA is implicated in Pompe disease, in which a deficiency in GAA results in the deficient processing of lysosomal glycogen. Accumulation of lysosomal glycogen occurs predominantly in skeletal, central nervous system, cardiac, and hepatic tissues. Infantile onset Pompe causes cardiomegaly, hypotonia, hepatomegaly, and death due to cardiorespiratory failure, usually before 2 years of age. Adult onset Pompe occurs as late as the second to sixth decade and usually involves only skeletal muscle. Treatments currently available include Genzyme's MYOZYME®/LUMIZYME® (alglucosidase alfa), which is a recombinant human alpha-glucosidase produced in CHO cells and administered by intravenous infusion.

Fabry disease, which has including mild late onset cases an overall estimated incidence of 1 in 3,000, is caused by defective lysosomal enzyme alpha-galactosidase A (GLA), which results in the accumulation of globotriaosylceramide within the blood vessels and other tissues and organs. Symptoms associated with Fabry disease include pain from nerve damage and/or small vascular obstruction, renal insufficiency and eventual failure, cardiac complications such as high blood pressure and cardiomyopathy, dermatological symptoms such as formation of angiokeratomas, anhidrosis or hyperhidrosis, and ocular problems such as cornea verticillata, spoke-like cataract, and conjunctival and retinal vascular abnormalities. Treatments currently available include Genzyme's FABRAZYME® (agalsidase beta), which is a recombinant human alpha-galactosidase A produced in CHO cells and administered by intravenous infusion; Shire's REPLAGAL™ (agalsidase alfa), which is a recombinant human alpha-galactosidase A produced in human fibroblast cells and administered by intravenous infusion; and Amicus's GALAFOLD™ (migalastat or 1-deoxygalactonojirimycin) an orally administered small molecule chaperone that shifts the folding of abnormal alpha-galactosidase A to a functional conformation.

Example 3 describes an animal model useful for measuring the efficacy of a multidomain therapeutic comprising an anti-human-TfR binding protein fused with a therapeutic agent in reducing glycogen accumulation in a tissue, particularly a CNS tissue. In some embodiments, such animal model comprises a non-human animal modified to express a human TfR protein as described herein, and further modified to comprise a knockout mutation of a gene encoding a lysosomal enzyme implicated in a lysosomal disorder, e.g., GAA in Pompe disease.

Accordingly, in some embodiments, an animal model as disclosed herein is modified to express a human TfR protein as described herein, and is further modified to comprise one or more additional genetic mutations such that the non-human animal further exhibits one or more symptoms of a disorder listed in Table 2. Methods of further modifying a non-human animal as described herein include, e.g., CRISPR-mediated deletion of a gene related to the disorder and other well-known recombinant DNA techniques.

In methods of screening anti-human-TfR binding protein based therapeutic agents, e.g., using a non-human animal as disclosed herein (which may be further modified to exhibit one or more symptoms of a disease—e.g., as listed in Table 2), a CRISPR/Cas system may be used to, e.g., insert the candidate anti-TfR binding protein into a locus for expression and/or knocking out a gene to create an animal model of a disease, whereby the animal disease model also expresses a human TfR protein or portion thereof. The methods and compositions disclosed herein can utilize nuclease agents such as Clustered Regularly Interspersed Short Palindromic Repeats (CRISPR)/CRISPR-associated (Cas) systems, zinc finger nuclease (ZFN) systems, or Transcription Activator-Like Effector Nuclease (TALEN) systems or components of such systems to modify a target genomic locus in a target gene such as a safe harbor gene (e.g., ALB) for insertion of a nucleic acid construct as disclosed herein. Generally, the nuclease agents involve the use of engineered cleavage systems to induce a double strand break or a nick (i.e., a single strand break) in a nuclease target site. Cleavage or nicking can occur through the use of specific nucleases such as engineered ZFNs, TALENs, or CRISPR/Cas systems with an engineered guide RNA to guide specific cleavage or nicking of the nuclease target site. Any nuclease agent that induces a nick or double-strand break at a desired target sequence can be used in the methods and compositions disclosed herein. The nuclease agent can be used to create a site of insertion at a desired locus (target gene) within a host genome, at which site the nucleic acid construct is inserted to express the polypeptide of interest (e.g., multidomain therapeutic protein). The polypeptide of interest (e.g., multidomain therapeutic protein) may be exogenous with respect to its insertion site or locus (target gene), such as a safe harbor locus from which polypeptide of interest is not normally expressed. Alternatively, the polypeptide of interest may be non-exogenous with respect to its insertion site, such as insertion into an endogenous locus encoding the polypeptide of interest to correct a defective gene encoding the polypeptide of interest.

In one example, the nuclease agent is a CRISPR/Cas system. In another example, the nuclease agent comprises one or more ZFNs. In yet another example, the nuclease agent comprises one or more TALENs. In a specific example, the CRISPR/Cas systems or components of such systems target an ALB gene or locus (e.g., ALB genomic locus) within a cell, or intron 1 of an ALB gene or locus within a cell. In a more specific example, the CRISPR/Cas systems or components of such systems target a human ALB gene or locus or intron 1 of a human ALB gene or locus within a cell.

CRISPR/Cas systems include transcripts and other elements involved in the expression of, or directing the activity of, Cas genes. A CRISPR/Cas system can be, for example, a type I, a type II, a type III system, or a type V system (e.g., subtype V-A or subtype V-B). The methods and compositions disclosed herein can employ CRISPR/Cas systems by utilizing CRISPR complexes (comprising a guide RNA (gRNA) complexed with a Cas protein) for site-directed binding or cleavage of nucleic acids. A CRISPR/Cas system targeting an ALB gene or locus comprises a Cas protein (or a nucleic acid encoding the Cas protein) and one or more guide RNAs (or DNAs encoding the one or more guide RNAs), with each of the one or more guide RNAs targeting a different guide RNA target sequence in the target genomic locus (e.g., ALB gene or locus).

CRISPR/Cas systems used in the compositions and methods disclosed herein can be non-naturally occurring. A non-naturally occurring system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes comprising a gRNA and a Cas protein that do not naturally occur together, employ a Cas protein that does not occur naturally, or employ a gRNA that does not occur naturally.

In some embodiments, a non-human animal expressing a human TfR protein or portion thereof as described herein is further modified by a CRISPR/Cas system as described herein to insert an anti-human-TfR antibody for expression in the non-human animal and/or to modify a gene associated with a disease listed in Table 2 such that the non-human animal expresses a human TfR protein or portion thereof and exhibits one or more symptoms of the disease.

In some embodiments, the toxicity in the animal may be measured as an adverse event in the animal, e.g., change in body weight, appetite, digestive changes, changes in blood cell counts, splenomegaly, histological changes of the organs, change in liver enzyme function, changes in urinalysis, organ toxicity, hemorrhage, dehydration, loss of fur and scruffiness, or other signs of morbidity. One measure may be determination of binding protein cross-reactivity with irrelevant antigens, which, in one embodiment, can be detected by organ histology, specifically detection of binding protein in tissues or cell types that are not known to express the antigen of interest.

Also described are various methods of using the genetically modified non-human animals described herein.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

TABLE 3
Description of Sequences.

| SEQ | | |
|---|---|---|
| ID NO | Type | Description |
| 1 | DNA | mTfrc-Coding Sequence-NCBI Gene ID 22042 |
| 2 | Protein | mTfR-UniProt ID Q62351 |
| 3 | DNA | hTfrc Coding Sequence-NCBI Gene ID 7037 |
| 4 | Protein | hTfR-UniProt ID P02786 |
| 5 | DNA | m/h TFRC CDS |
| 6 | DNA | m/h TFRC CDS |
| 7 | DNA | mouse 5′ arm |
| 8 | DNA | mouse 3′ arm |
| 9 | DNA | 7228 allele |
| 10 | DNA | 7229 allele |
| 11 | DNA | 7228mTU fwd: CCCAAGAGGTTTAATGGAAGACTC |
| 12 | DNA | 7228mTU rev: GGCCACTCATTCTTACCAATCAAG |
| 13 | DNA | 7228mTU probe: TGCTTTGCAGCTATTGCACTAGTCA |
| 14 | DNA | 7228hTU fwd: CTGACTGAGAAGACCAGTGTGA |
| 15 | DNA | 7228hTU rev: AGGGTGTTCTCCTTTCAAACCAA |
| 16 | DNA | 7228hTU probe: CTAGCCCTTGACTGGGCCCA |
| 17 | DNA | 7228mTD fwd: GGAGGGTCAACGTGGTAGTTG |
| 18 | DNA | 7228mTD rev: AAGGGCATACAGCTCAATGGA |
| 19 | DNA | 7228mTD probe: TAGTTGGAGATTGCCCCGTCTCC |
| 20 | DNA | 7228hTD fwd: GCATGTGGCATGTTCATCGTA |
| 21 | DNA | 7228hTD rev: AGCACGTACTGGCTTGATAGG |
| 22 | DNA | 7228hTD probe: ATGAATACAGGGCATGCATTTTGCAGC |
| 23 | DNA | TfR exon 1 5′ primer |
| 24 | DNA | TfR exon 1 3′ primer |
| 25 | Protein | Mouse/Human TfR protein from 7228 or 7229 allele |
| 26 | Protein | Human TfR Cytoplasmic Domain (amino acids 1-67 of SEQ ID |
| | | NO: 4 or SEQ ID NO: 25) |
| 27 | Protein | Human TfR Transmembrane Domain (amino acids 68-88 of SEQ |
| | | ID NO: 4 or SEQ ID NO: 25) |
| 28 | Protein | Human TfR Extracellular Domain (amino acids 89-760 of SEQ ID |
| | | NO: 4 or SEQ ID NO: 25_ |
| 29 | DNA | 9251mGU: GGCAGCATGAATACTTCTGA |
| 30 | DNA | 9251mGU3: ACCACGGAGTTCGAACAGAG |
| 31 | DNA | 9251mGD3: AATATACCCTACCTGTGGCA |
| 32 | DNA | 9251mGD4: TTGGTGGAGGAGACACGCCA |
| 33 | DNA | 9251mTU Fwd: CTCATGCTTCGGGAGTTAATGCT |
| 34 | DNA | 9251mTU Probe: CTCATGCTTCGGGAGTTAATGCT |
| 35 | DNA | 9251mTU Rev: GGTCGGTACGTCTTCCACAGT |
| 36 | DNA | 9251mretU Fwd: GTGTGTCTGGTGACCCTGA |
| 37 | DNA | 9251mretU Probe: AACCGTCCTCTGTGCTGAGCCC |
| 38 | DNA | 9251mretU Rev: GTGGGCCTTCTGGAAATGG |
| 39 | DNA | 9251mTM Fwd: GGGACCAACCTAGCTCACATCT |
| 40 | DNA | 9251mTM Probe: TGCCTCTTCCAGGACATGAACGA |
| 41 | DNA | 9251mTM Rev: CCCTGCTGAGAGCCTCTAAC |
| 42 | DNA | 9251mretD3: GCGATGGAGCCGTGGATT |
| 43 | DNA | 9251mretD3: ACCTCAGGCTGTTACAGGGCTG |
| 44 | DNA | 9251mretD3: CCCTCCCAGTTCAGGTCTCA |
| 45 | DNA | 9251mTGD: GCCTGGCCATCCCTGTCT |
| 46 | DNA | 9251mTGD: CACTGCTGATGGGAGAGCTGTTTCA |
| 47 | DNA | 9251mTGD: GCCTCTGTAAACGACGGACTCT |
| 48 | Protein | Mouse GAA |
| 49 | DNA | Mouse Gaa |
| 50 | DNA | Mouse Gaa−/− allele |
| 51 | DNA | Mouse Gaa−/− allele |
| 52 | Protein | Mature GAA |

### EXAMPLES

### Example 1. Generation and Analysis of TfR Humanized Mice (Tfrc^{hum})

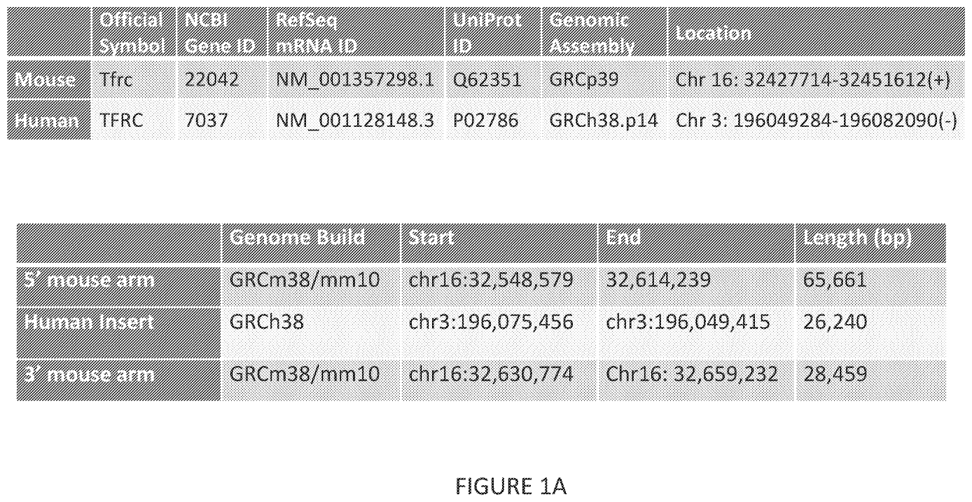

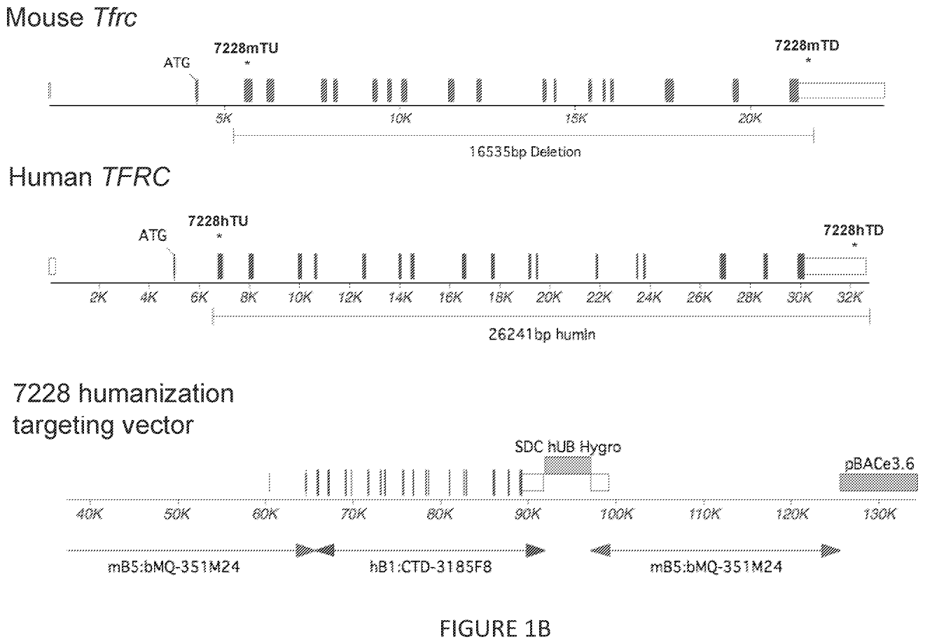

FIG. 1A provides identifying information about mouse and human transferrin receptor gene (TfR) and FIG. 1B provides an illustrative schematic (not to scale) of the mouse and human transferrin receptor genes, and the humanized mouse transferrin receptor targeting vector. As shown in FIG. 1B, exon 1 of both human and mouse TFRC is non-coding. Exon 2 of human and mouse TFRC comprises coding sequences and may be considered coding exon 1. Since exon 2/coding exon 1 of mouse and human TFRC encode identical amino acid sequences, exon 2/coding exon 1 of the mouse TFRC locus may be or may not be included in the humanization.

The TFRC targeting construct was designed as follows. A bacterial artificial chromosome containing the complete mouse Tfrc genomic sequence was modified to humanize the Tfrc locus. As depicted in FIG. 1B, mouse Tfrc locus was deleted starting within intron 2, through part of the Tfrc 3′ UTR, such that noncoding exon 1, intron 1, exon 2 (coding exon 1), and the 5′ end of intron 2 were preserved. The deletion removed the 3′ 326 bp of mouse intron 2, exons 3-18 (and the intervening introns), intron 18, and the mouse exon 19 coding sequence with part of the 3′ UTR. The 3′ 2021 bp of mouse 3′ UTR were left intact. In place of this deletion, a human TFRC genomic sequence including 195 bp of the 3′ end of human intron 2, exons 3-18 (and the intervening introns), intron 19, the coding sequence of exon 19, and all but the last 32 bp of human 3′ UTR were inserted. The part of coding exon 2, intron 2, coding exons 3-18 (and intervening introns), and 82 bp of 3′ untranslated region (UTR) mouse Tfrc were replaced with the described human TFRC sequence comprising a coding exon 1 sequence minus the first 15 bp (this start sequence remains mouse), intron 1, coding exons 2-4 (and intervening introns), complete 3′ UTR and an additional 158 bp after the 3′ UTR of human TFR. See FIGS. 1A-1B. A self-deleting hygromycin resistance cassette was inserted downstream of the human sequence, with the remainder of the mouse 3′ UTR to follow. This targeting vector was then electroporated into a 50% C57Bl/6NTac/50% 129SvEvTac embryonic stem cell line. Successfully targeted clones were identified by TaqMan analysis. Tfrc^{hum} mice were generated using the VelociGene© method (Valenzuela 2003 Nat Biotech PMID:12730667; Poueymirou 2007 Nat Biotech PMID:17187059) and backcrossed to C57Bl/6NTac as needed. Antibiotic resistance cassettes were removed in the F0 male germline using self-deleting technology. Loss-of-allele assays were performed to detect loss of the endogenous mouse allele, gain-of-allele assays were performed to detect gain of the humanized allele using primers and probes to detect the absence or presence of 7228mTU, 7728mTD, 7228hTU, and 7728hTD sequences in the 7228 allele (TfR humanization comprising the hygromycin self-deleting cassette) or the 7229 allele (TfR humanization after deletion of the hygromycin self-deleting cassette). See, Table 4.

TABLE 4

| Mouse TaqMan Loss of Allele Assays | | | Human Taqman Loss of Allele Assays | | |
|---|---|---|---|---|---|
| 7228mTU | Fwd | CCCAAGAGGTTTAATG | 7228hTU | Fwd | CTGACTGAGAAGACCAGT |
| | | GAAGACTC | | | GTGA |
| | | (SEQ ID NO 11) | | | (SEQ ID NO: 14) |
| | Probe | TGCTTTGCAGCTATTGC | | Probe | CTAGCCCTTGACTGGGCCC |
| | (BHQ) | ACTAGTCA | | (BHQ) | A |
| | | (SEQ ID NO: 13) | | | (SEQ ID NO: 16) |
| | Rev | GGCCACTCATTCTTACC | | Rev | AGGGTGTTCTCCTTTCAAA |
| | | AATCAAG | | | CCAA |
| | | (SEQ ID NO: 12) | | | (SEQ ID NO: 15) |
| 7228mTD | Fwd | GGAGGGTCAACGTGGT | 7228hTD | Fwd | GCATGTGGCATGTTCATCG |
| | | AGTTG | | | TA |
| | | (SEQ ID NO: 17) | | | (SEQ ID NO: 20) |
| | Probe | TAGTTGGAGATTGCCCC | | Probe | ATGAATACAGGGCATGCAT |
| | (BHQ) | GTCTCC | | (BHQ) | TTTGCAGC |
| | | (SEQ ID NO: 19) | | | (SEQ ID NO: 22) |
| | Rev | AAGGGCATACAGCTCA | | Rev | AGCACGTACTGGCTTGATA |
| | | ATGGA | | | GG |
| | | (SEQ ID NO: 18) | | | (SEQ ID NO: 21) |

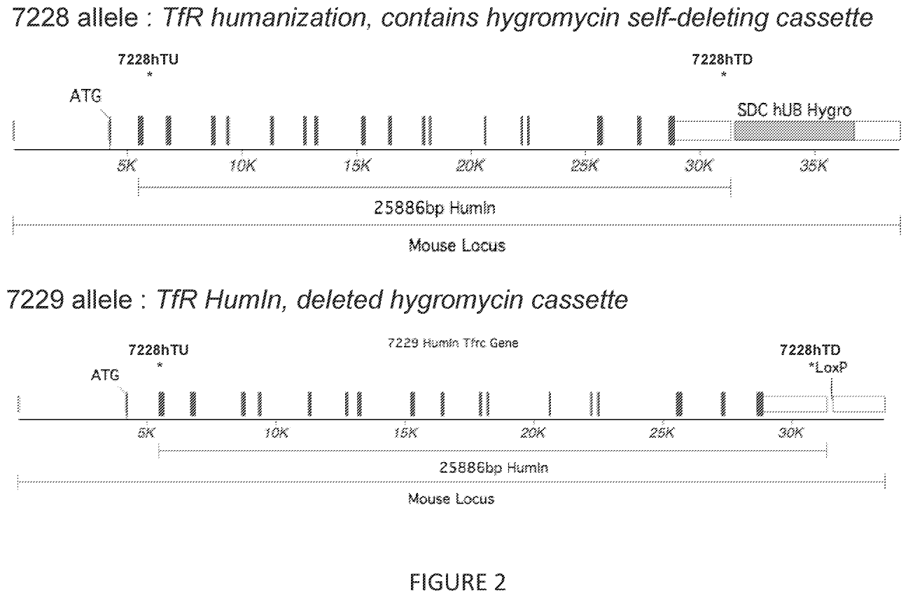

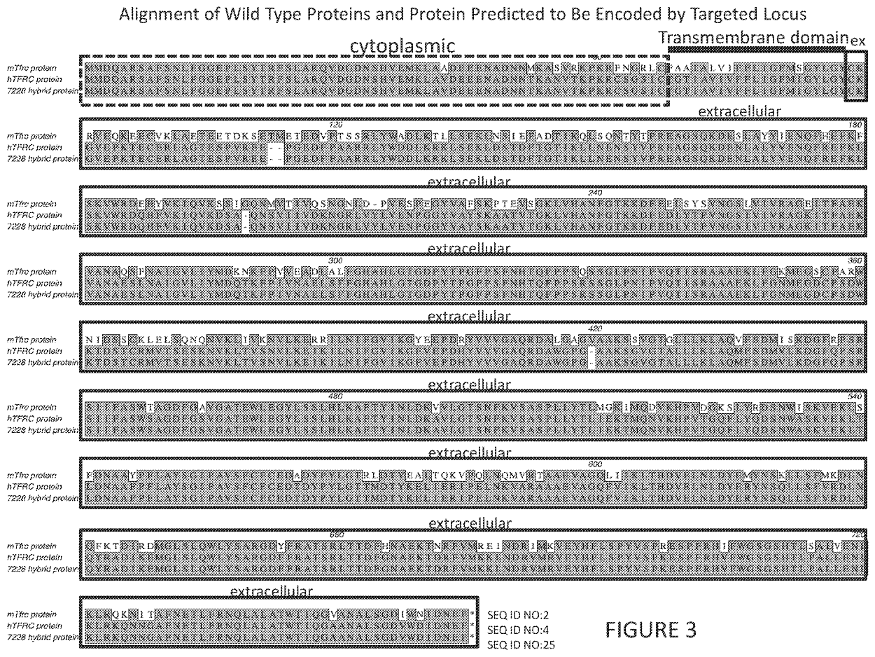

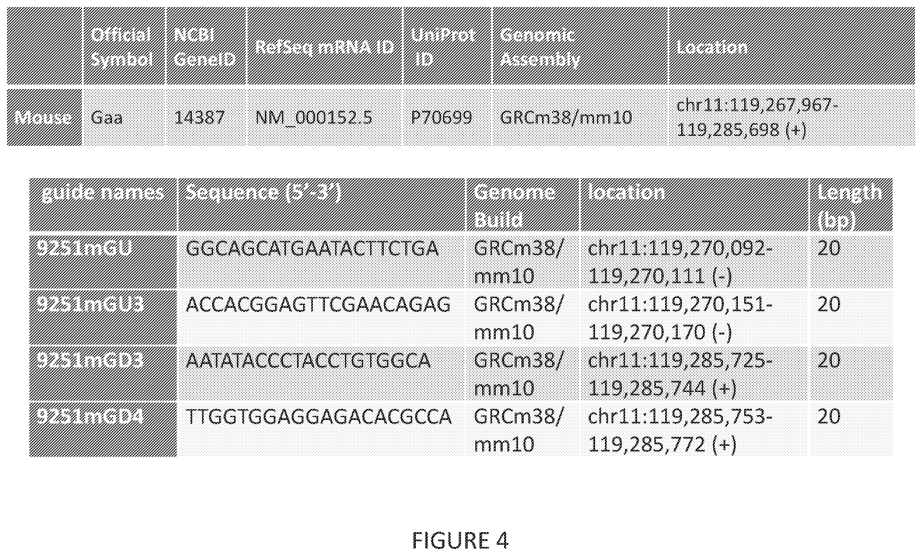

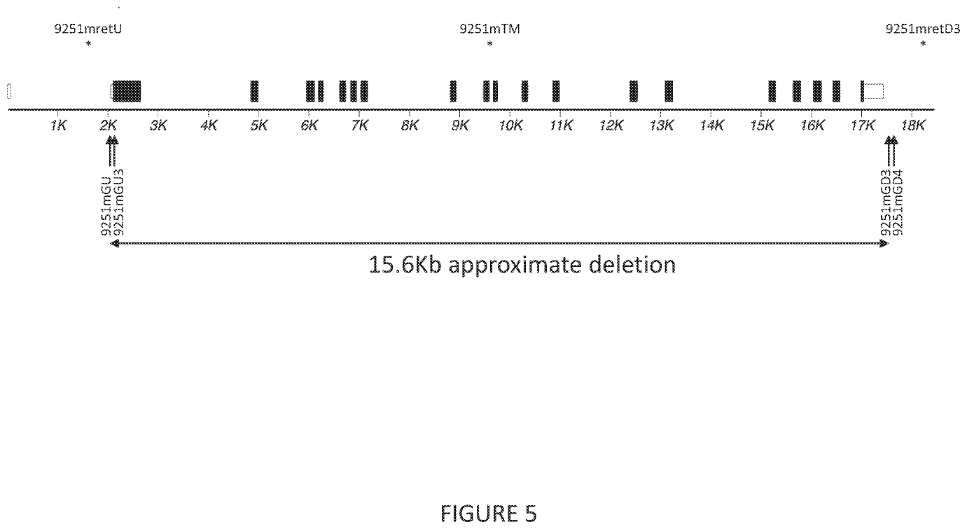

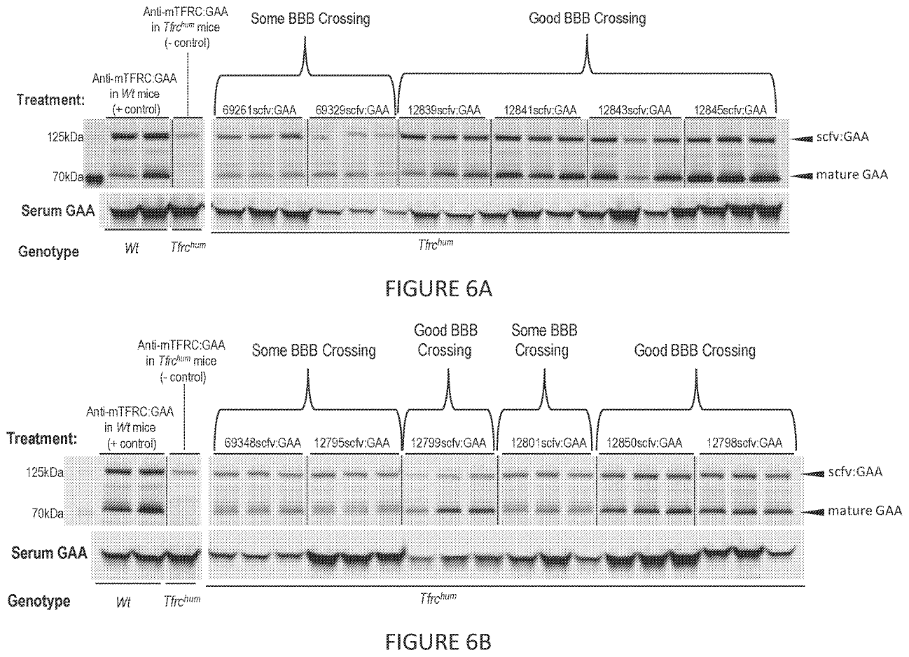

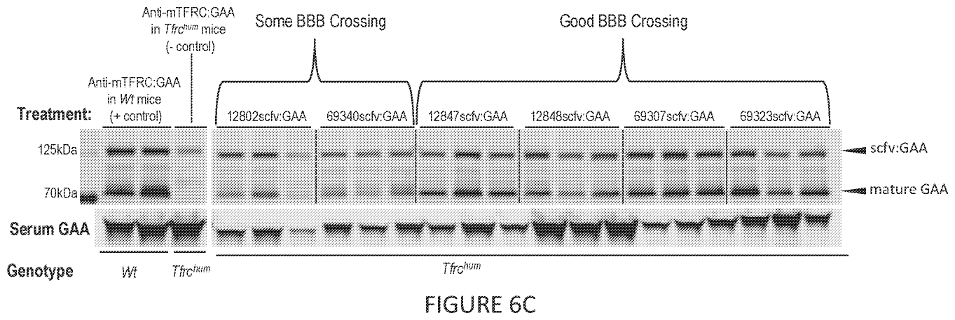

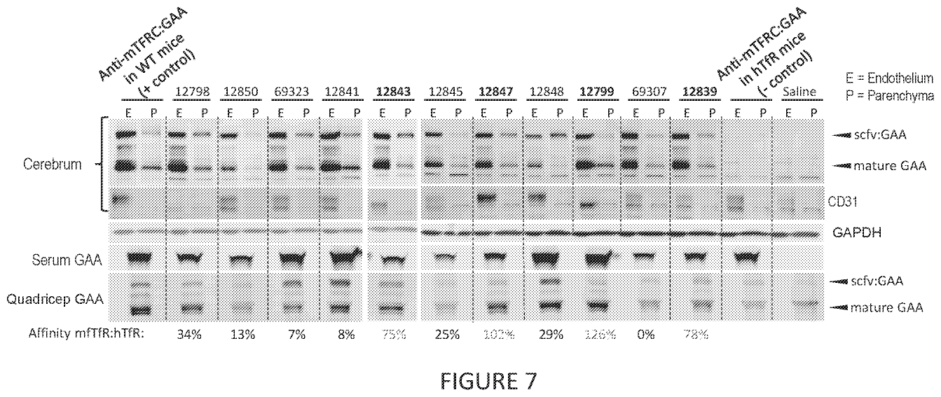

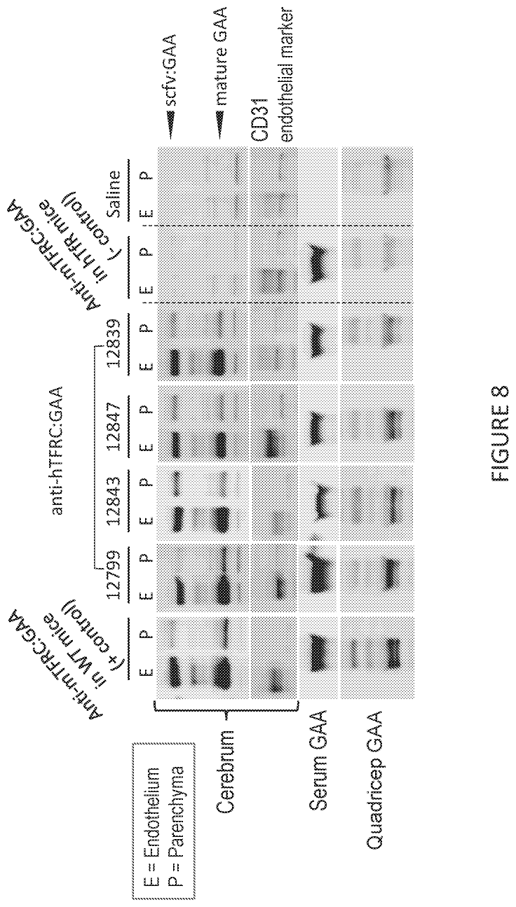

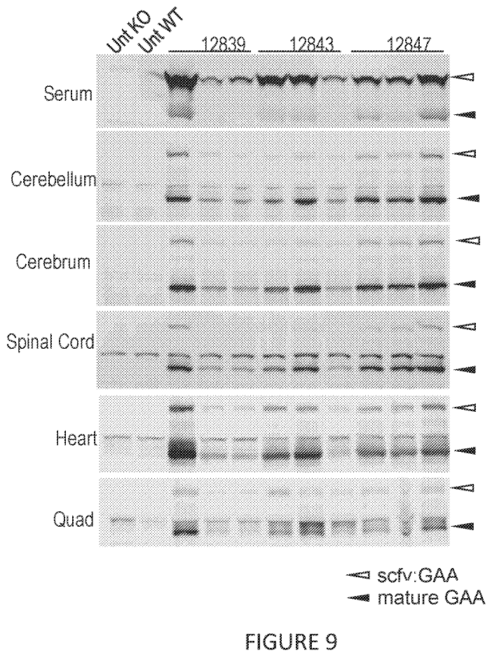

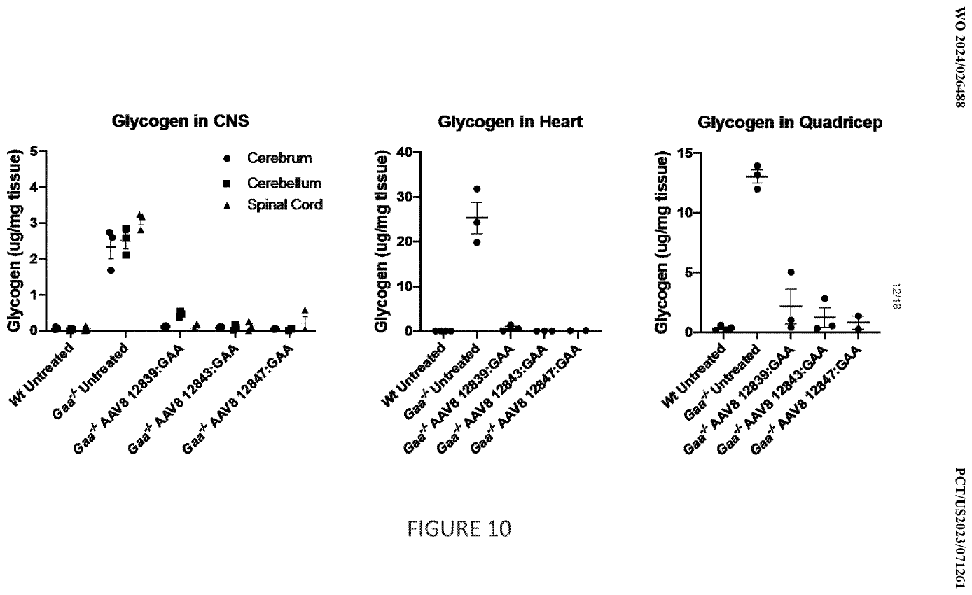

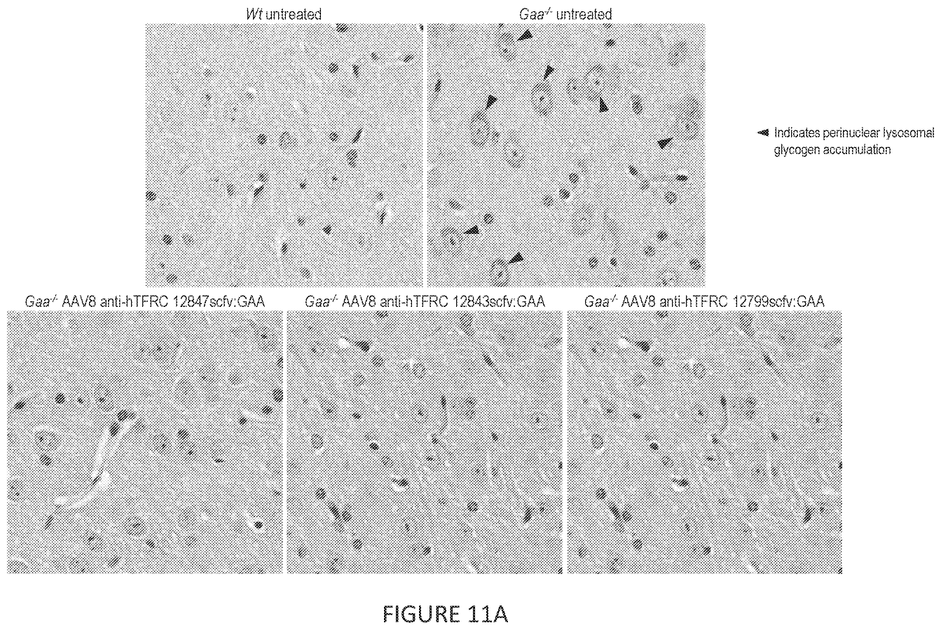

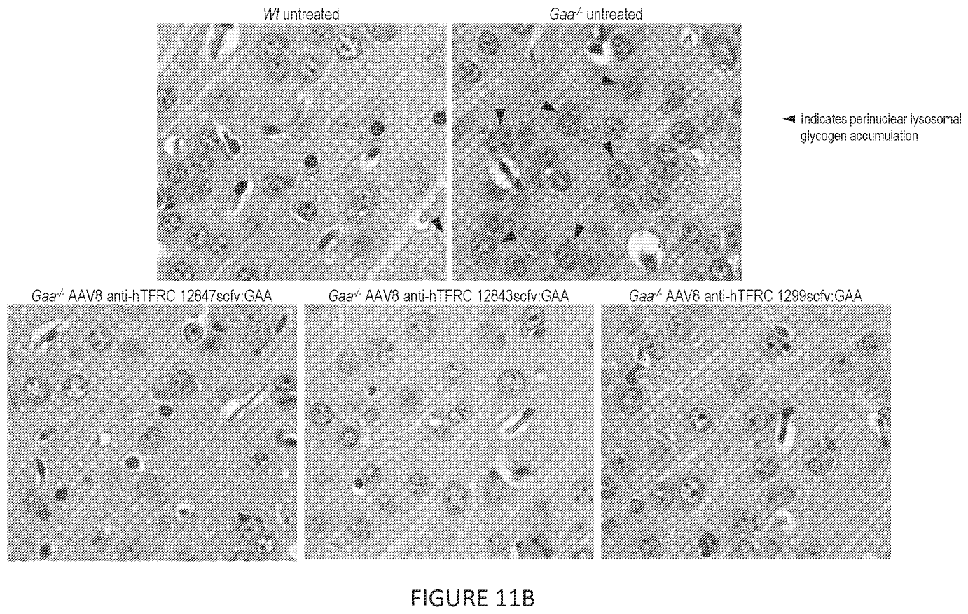

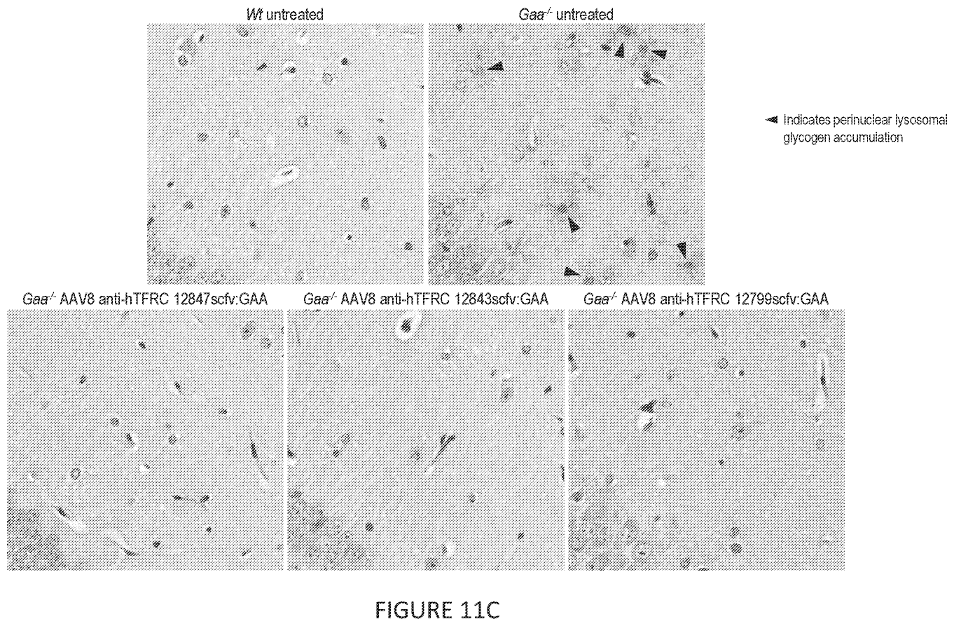

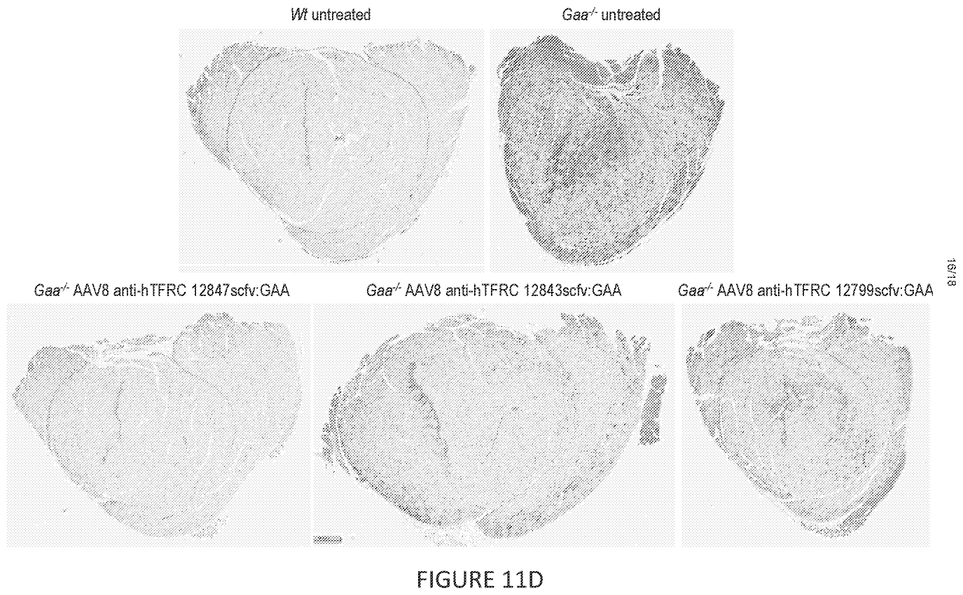

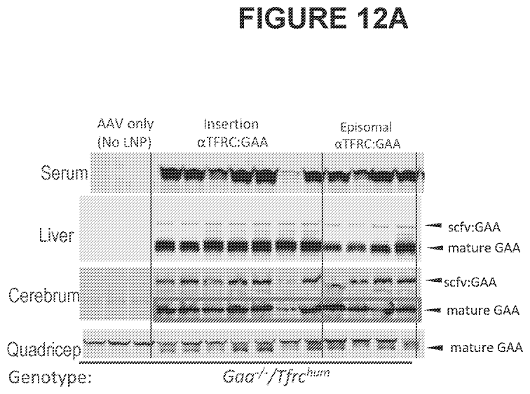

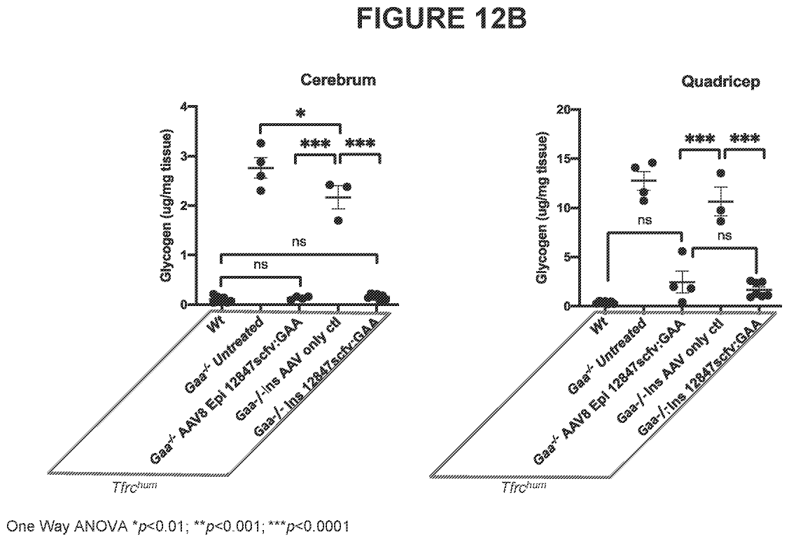

FIG. 2 provides schematic illustrations (not to scale) of the modified allele before and after deletion of the self-deleting hygromycin resistance cassette. F0 mice were bred to homozygosity to generate Tfrc^{hum/hum} mice, also referred to herein as Tfrc^{hum} mice.

### Gene Expression Analysis:

To validate that Tfrc^{hum} mice expressed TfR at physiological levels and had normal iron homeostasis, quantified expression of TfR in tissues, serum markers, tissue iron content, and transferrin in tissue from Tfrc^{hum} mice were compared to wildtype (WT) mice. Overall, the results indicated that TfR expression and iron homeostasis was normal in the Tfrc^{hum} mice.

Specifically, 6 month old WT mice (11 males, 4 females) and Tfrc^{hum} mice (10 males, 8 females) were analyzed. Tissues were dissected from mice immediately after sacrifice by CO₂ asphyxiation, snap frozen in liquid nitrogen, and stored at −80° C.

Tfr RNA Quantification by qPCR:

Total RNA was isolated from tissues with Trizol following manufacturer protocol (ThermoFisher 15596026). Tfr RNA was quantified by Tagman qPCR (ThermoFisher) following standard protocols using universal primers to exon 1 that amplify from both WT and and Tfrc^{hum} mice (GCTGCATTGCGGACTGTAGA; SEQ ID NO:23/TCCATCATTCTCAGCTGCTACAA; SEQ ID NO:24). ΔΔCT values were calculated relative to the WT male group. See, Table 5. The results indicate that Tfr RNA was detected in the humanized Tfr mice.

### Serum Assays:

Blood was collected from mice by cardiac puncture immediately following CO₂ asphyxiation and serum was separated using serum separator tubes (BD Biosciences, 365967). Serum iron and Total Iron Binding Content (TIBC) were quantified using standard protocols. Serum hepcidin was quantified by ELISA kit (Intrinsic Life Sciences SKU HMC-001). See, Table 6. The results indicate that the humanized Tfr mice displayed iron homeostasis similar to wild type mice.

### Tissue Iron Content:

Wet tissue was weighed to achieve uniformity and then dried for 72 hours in an open tube at 56° C. Tissue was then placed in digestion buffer (10% Tricloroacetic acid and 37% HCL) and heated at 65° C. for 48 hours. To assay iron content, the supernatant was placed in a 96 well plate and incubated in a color development solution (Thioglycolic acid, bathophenanthroline acid and sodium acetate). Absorbance was read on a Spectramax i3 by Molecular Devices and Graph Pad Prism was used to interpolate the sample absorbance values read against a standard curve to calculate iron content in the whole piece of tissue. Iron content was then calculated based on dry weight. See, Table 7. The results indicate that the humanized Tfr mice displayed iron homeostasis similar to wild type mice.

### Transferrin ELISA:

All tissues were homogenized using a Fastprep-24 5G from MP Biomedicals. Prior to homogenization, tissues were placed in RIPA buffer with phosphatase and HALT protease inhibitors (ThermoFisher), homogenized with their organ specific protocol and then centrifuged to pellet debris. The supernatant was collected and assayed for total protein using a Pierce BCA Protein Assay Kit. Absorbance was measured on a Spectramax i3 by Molecular Devices. Once total protein was measured, all samples were diluted to match the least concentrated sample so loading would be uniform for the ELISA. Kits obtained from Abcam were used to measure the presence of total transferrin in tissue homogenate (Abcam ab157724). Plates were run in accordance with the supplied protocol using the provided reagents and absorbance was read on a Spectramax i3 by Molecular Devices. Graph Pad Prism was used to interpolate the sample absorbance values read against a standard curve. See, Table 8. The results indicate that Tfr protein was detected in the humanized Tfr mice.

TABLE 5
Tfr RNA quantification in WT and Tfrc^{hum} mice

| Genotype | Sex | Liver Tfr | Quadricep Tfr | Brain Tfr |
|---|---|---|---|---|
| Wt | M | 1.02 ± 0.21 | 1.10 ± 0.53 | 1.02 ± 0.21 |
| Wt | F | 1.11 ± 0.64 | 0.60 ± 0.17 | 1.03 ± 0.13 |
| Tfrc^{hum} | M | 1.14 ± 0.28 | 1.02 ± 0.39 | 1.86 ± 0.35* |
| Tfrc^{hum} | F | 0.75 ± 0.22 | 0.43 ± 0.93 | 1.88 ± 0.25* |

All values are ΔΔCT vs. Wt males group, mean ± SD, n = 4-11 per group.
One Way ANOVA
*p < 0.001 vs. Wt sex-matched group.

TABLE 6
Serum iron markers in WT and Tfrc^{hum} mice

| | | | | Serum |
|---|---|---|---|---|
| | | Serum Iron | Serum TIBC | Hepcidin |
| Genotype | Sex | ug/dL | ug/dL | ng/mL |
| Wt | M | 146.73 ± 20.30 | 360.18 ± 27.02 | 416.73 ± 133.04 |
| Wt | F | 125.50 ± 9.04 | 342.25 ± 22.25 | 436.35 ± 143.28 |
| Tfrc^{hum} | M | 173.00 ± 12.77* | 351.20 ± 21.94 | 415.86 ± 101.27 |
| Tfrc^{hum} | F | 156.75 ± 14.18* | 353.50 ± 17.03 | 523.30 ± 175.70 |

All values are mean ± SD, n = 4-11 per group. All values are within normal physiological range.
One Way ANOVA
*p < 0.05 vs. Wt sex-matched group.

TABLE 7
Tissue iron quantification in WT and Tfrc^{hum} mice

| Genotype | Sex | Liver | Spleen |
|---|---|---|---|
| Wt | M | 307.03 ± 32.74 | 1666.38 ± 239.18 |
| Wt | F | 507.45 ± 110.45 | 1833.12 ± 173.36 |
| Tfrc^{hum} | M | 300.00 ± 33.77 | 1818.44 ± 276.86 |
| Tfrc^{hum} | F | 638.46 ± 139.03 | 1695.96 ± 140.02 |

All values are ug/g dry tissue, mean ± SD, n = 4-11 per group.
Values are non-significant (One Way ANOVA vs. Wt sex-matched group).

TABLE 8
Transferrin protein in WT and Tfrc^{hum} mice (ELISA)

| Genotype | Sex | Serum | Liver | Cerebrum |
|---|---|---|---|---|
| Wt | M | 1191.28 ± 137.03 | 32.61 ± 9.87 | 7.35 ± 1.30 |
| Wt | F | 1270.81 ± 138.42 | 27.01 ± 13.22 | 6.33 ± 0.93 |
| Tfrc^{hum} | M | 1251.40 ± 113.59 | 32.97 ± 7.26 | 7.92 ± 1.63 |
| Tfrc^{hum} | F | 1425.89 ± 290.77 | 40.17 ± 8.22 | 8.26 ± 2.08 |

All values are ug/mL homogenate normalized to protein content; mean ± SD, n = 4-11 per group.
Values are non-significant (One Way ANOVA vs. Wt sex-matched group).

To validate the function of the human TfR protein expressed by Tfrc^{hum} or Tfrc^{hum}Gaa^{-/-}mice, anti-hTfR binding proteins fused to the mature peptide of human acid alpha-glucosidase (GAA) were used. The acid alpha-glucosidase hydrolyzes alpha-1,4 linkages between the D-glucose units of glycogen, maltose, and isomaltose. The amino acid sequence for mature peptide of human alpha-glucosidase used in the fusion proteins is set forth as SEQ ID NO:52.

Tfrc^{hum} mice were injected with DNA plasmids expressing various anti-hTFRC antibodies in an anti-hTFRCscfv:2xG4S:hGAA format that is under the liver-specific mouse TTR promoter. Mice received 50 μg of DNA in 0.9% sterile saline diluted to 10% of the mouse's body weight (0.1 mL/g body weight). 48 hours post-injection, tissues were dissected from mice immediately after sacrifice by CO₂ asphyxiation, snap frozen in liquid nitrogen, and stored at −80° C.

Tissue lysates were prepared by lysis in RIPA buffer with protease inhibitors (1861282, Thermo Fisher, Waltham, MA, USA). Tissue lysates were homogenized with a bead homogenizer (FastPrep5, MP Biomedicals, Santa Ana, CA, USA). Cells or tissue lysates were run on SDS-PAGE gels using the Novex system (LifeTech Thermo, XP04200BOX, LC2675, LC3675, LC2676). Gels were transferred to low-fluorescence polyvinylidene fluoride (PVDF) membrane (IPFLO7810, LI-COR, Lincoln, NE, USA) and stained with Revert 700 Total Protein Stain (TPS; 926-11010 LI-COR, Lincoln, NE, USA), followed by blocking with Odyssey blocking buffer (927-500000, LI-COR, Lincoln, NE, USA) in Tris buffer saline with 0.1% Tween 20 and staining with antibodies against GAA (abl37068, Abcam, Cambridge, MA, USA), or anti-GAPDH (ab9484, Abcam, Cambridge, MA, USA) and the appropriate secondary (926-32213 or 925-68070, LI-COR, Lincoln, NE, USA). Blots were imaged with a LI-COR Odyssey CLx.

Protein band intensity was quantified in LI-COR Image Studio software. The quantification of the mature 77 kDa GAA band for each sample was determined by first normalizing to the lane's TPS signal, then normalizing to GAA levels in the serum (loading control and liver expression control, respectively). Values were then compared to the positive control group anti-mouse TFRCscfv:hGAA in WT mice, and negative control group anti-mTFRCscfv:hGAA in Tfrc^{hum} mice (FIGS. 6A-6C, Table 9). The results show that anti-human TfR antibody clones deliver GAA to the cerebrum of Tfrc^{hum} mice.

TABLE 9
Quantification of mature hGAA protein in brain homogenate
from mice treated HDD with anti-hTFRCscfv:hGAA plasmids.

| | | Mature hGAA protein in |
|---|---|---|
| | | brain (normalized to |
| Treatment group | Genotype | positive control) |
| anti-mTFRCscfv:hGAA (positive | Wt | 1.00 ± 0.43* |
| control) | | |
| anti-mTFRCscfv:hGAA (negative | Tfrc^{hum} | 0.02 ± 0.03 |
| control) | | |
| 69261scfv:hGAA | Tfrc^{hum} | 0.67 ± 0.50 |
| 69307scfv:hGAA | Tfrc^{hum} | 1.08 ± 0.19 |
| 69323scfv:hGAA | Tfrc^{hum} | 0.91 ± 0.46 |
| 69329scfv:hGAA | Tfrc^{hum} | 0.65 ± 0.13 |
| 69340scfv:hGAA | Tfrc^{hum} | 0.55 ± 0.58 |
| 69348scfv:hGAA | Tfrc^{hum} | 0.50 ± 0.05 |
| 12795scfv:hGAA | Tfrc^{hum} | 0.27 ± 0.20 |
| 12798scfv:hGAA | Tfrc^{hum} | 0.72 ± 0.42 |
| 12799scfv:hGAA | Tfrc^{hum} | 1.05 ± 0.51* |
| 12801 scfv:hGAA | Tfrc^{hum} | 0.49 ± 0.18 |
| 12802scfv:hGAA | Tfrc^{hum} | 0.29 ± 0.27 |
| 12839scfv:hGAA | Tfrc^{hum} | 1.29 ± 0.27** |
| 12841scfv:hGAA | Tfrc^{hum} | 1.72 ± 0.06*** |
| 12843scfv:hGAA | Tfrc^{hum} | 1.79 ± 0.85*** |
| 12845scfv:hGAA | Tfrc^{hum} | 3.08 ± 0.92*** |
| 12847scfv:hGAA | Tfrc^{hum} | 1.24 ± 0.30 |
| 12848scfv:hGAA | Tfrc^{hum} | 0.59 ± 0.16 |
| 12850scfv:hGAA | Tfrc^{hum} | 0.47 ± 0.05 |

Data were quantified from western blot as arbitrary units (FIGS. 9A-9C).
All values are mean ± SD, n = 3-6 per group.
One Way ANOVA vs. negative control anti-mTFRCscfv:hGAA in Tfrc^{hum} mice;
*p < 0.05;
**p < 0.005;
***p < 0.0001.

Capillary depletion of brain samples following hydrodynamic delivery (HDD) of anti-hTFRCscfv:hGAA plasmids. Select anti-hTFRCscfv:hGAA (see, e.g., Table 9) were tested in a secondary screen in Tfrc^{hum} mice to determine whether hGAA was present in the brain parenchyma, and not trapped in the BBB endothelial cells. Four scFvs (12799, 12839, 12843, and 12847) were selected from this screen based on mature hGAA in the parenchyma fraction on western blot, as well as high affinity to cynomolgus TFRC.

Forty-eight hours following HDD, mice were perfused with 30 mL 0.9% saline immediately after sacrifice by CO₂ asphyxiation. A 2 mm coronal slice of cerebrum was taken between bregma and −2 mm bregma and placed in 700 μL physiological buffer (10 mM HEPES, 4 mM KCl, 2.8 mM CaCl₂, 1 mM MgSO₄, 1 mM NaH₂PO₄, 10 mM D-glucose in 0.9% saline pH 7.4) on ice. Brain slices were gently homogenized on ice with a glass dounce homogenizer. An equivalent volume of 26% dextran (MW 70,000 Da) in physiological buffer was added (final 13% dextran) and homogenized 10 more strokes. Parenchyma (supernatant) and endothelial (pellet) fractions were separated by centrifugation at 5,400 g for 15 min at 4° C. Anti-hGAA western blot was performed on fractions as detailed above (FIG. 7, Table 10). Blots were also probed with anti-CD31 endothelial marker (Abcam ab182982). The data show a subset of anti-hTfR antibody clones deliver mature GAA to the brain parenchyma in scfv:GAA format.

TABLE 10
Quantification of mature hGAA protein in brain parenchyma fractions and BBB
endothelial fractions of mice treated HDD with anti-hTFRCscfv:hGAA plasmids.

| | | | Mature hGAA | |
|---|---|---|---|---|
| | | | protein in | |
| | | Mature hGAA | brain | Affinity to |
| | | protein in brain | endothelium | mfTFRC |
| | | parenchyma | (normalized to | (% of |
| | | (normalized to | positive | hTFRC |
| Treatment group | Genotype | positive control) | control) | binding) |
| anti- | Wt | 1.00 | 5.82 | ND |
| mTFRCscfv:hGAA | | | | |
| (positive control) | | | | |
| anti- | Tfrc^{hum} | 0.00 | 0.01 | ND |
| mTFRCscfv:hGAA | | | | |
| (negative control) | | | | |
| 69307scfv:hGAA | Tfrc^{hum} | 1.24 | 10.73 | 0% |
| 69323scfv:hGAA | Tfrc^{hum} | 0.62 | 4.18 | 7% |
| 12798scfv:hGAA | Tfrc^{hum} | 0.91 | 8.37 | 34% |
| 12799scfv:hGAA | Tfrc^{hum} | 0.44 | 3.99 | 126% |
| 12839scfv:hGAA | Tfrc^{hum} | 0.55 | 0.84 | 78% |
| 12841scfv:hGAA | Tfrc^{hum} | 0.78 | 4.23 | 8% |
| 12843scfv:hGAA | Tfrc^{hum} | 1.13 | 12.99 | 75% |
| 12845scfv:hGAA | Tfrc^{hum} | 2.04 | 13.06 | 25% |
| 12847scfv:hGAA | Tfrc^{hum} | 0.60 | 4.96 | 102% |
| 12848scfv:hGAA | Tfrc^{hum} | 0.17 | 1.24 | 29% |
| 12850scfv:hGAA | Tfrc^{hum} | 0.22 | 2.25 | 13% |

hGAA protein was quantified from western blot as arbitrary units (FIG. 7). n = 1 per group.
Affinity to cynomolgus macaque TFRC Luminex data, calculated as percent of binding to hTFRC: (mfTFRC binding ÷ hTFRC binding) × 100.

TABLE 11
Quantification of hGAA protein in quadricep of mice
treated HDD with anti-hTFRCscfv:hGAA plasmids.

| | | hGAA protein in quadricep |
|---|---|---|
| Treatment group | Genotype | (normalized to positive control) |
| Saline (vehicle) | Tfrc^{hum} | 0.38 ± 0.25 |
| anti-mTFRCscfv:hGAA (positive | Wt | 1.07 ± 0.27 |
| control) | | |
| anti-mTFRCscfv:hGAA (negative | Tfrc^{hum} | 0.56 ± 0.17 |
| control) | | |
| 69307scfv:hGAA | Tfrc^{hum} | 0.58 ± 0.18 |
| 69323scfv:hGAA | Tfrc^{hum} | 1.10 ± 0.19 |
| 12798scfv:hGAA | Tfrc^{hum} | 1.33 ± 0.56 |
| 12799scfv:hGAA | Tfrc^{hum} | 0.67 ± 0.18 |
| 12839scfv:hGAA | Tfrc^{hum} | 1.80 ± 0.18 |
| 12841scfv:hGAA | Tfrc^{hum} | 1.15 ± 0.12 |
| 12843scfv:hGAA | Tfrc^{hum} | 1.78 ± 0.43 |
| 12845scfv:hGAA | Tfrc^{hum} | 1.70 ± 1.33 |
| 12847scfv:hGAA | Tfrc^{hum} | 7.74 ± 9.42 |
| 12848scfv:hGAA | Tfrc^{hum} | 0.82 ± 0.18 |
| 12850scfv:hGAA | Tfrc^{hum} | 0.76 ± 0.34 |

Data were quantified from western blot as arbitrary units (FIG. 7).
All values are mean ± SD, n = 2-4 per group.

Capillary depletion of mouse brain samples following liver-depot AAV8 anti-hTFRCscfv:hGAA treatment. To confirm the HDD screen findings in a more long-term treatment model, Tfrc^{hum} mice were treated with selected anti-hTFRCscfv:GAA delivered as episomal liver depot AAV8 anti-hTFRCscfv:GAA under the TTR promoter. All four anti-hTFRCscfv:GAA delivered mature hGAA to the brain parenchyma when delivered as AAV8 (FIG. 8).

AAV production and in vivo transduction. Recombinant AAV8 (AAV2/8) was produced in HEK293 cells. Cells were transfected with three plasmids encoding adenovirus helper genes, AAV8 rep and cap genes, and recombinant AAV genomes containing transgenes flanked by AAV2 inverted terminal repeats (ITRs). On day 5, cells and medium were collected, centrifuged, and processed for AAV purification. Cell pellets were lysed by freeze-thaw and cleared by centrifugation. Processed cell lysates and medium were overlaid onto iodixanol gradients columns and centrifuged in an ultracentrifuge. Virus fractions were removed from the interface between the 40% and 60% iodixanol solutions and exchanged into 1×PBS with desalting columns. AAV vg were quantified by ddPCR. AAVs were diluted in PBS+0.001% F-68 Pluronic immediately prior to injection. Tfrc^{hum} mice were dosed with 3e12 vg/kg body weight in a volume of ˜100 μL. Mice were sacrificed 4 weeks post injection and capillary depletion and western blotting were performed as described above (FIG. 8, Table 12). The data show that four selected anti-hTfR antibody clones deliver mature GAA to the brain parenchyma in scfv:GAA format (AAV8 episomal liver depot gene therapy).

TABLE 12
Quantification of mature hGAA protein in brain parenchyma
fractions and BBB endothelial fractions of mice treated
with liver-depot AAV8 anti-hTFRCscfv:hGAA.

| | | Mature hGAA protein in | Mature hGAA protein |
|---|---|---|---|
| | | brain parenchyma | in brain endothelium |
| | | (normalized to positive | (normalized to positive |
| Treatment group | Genotype | control) | control) |
| anti- | Wt | 1.00 | 1.00 |
| mTFRCscfv:hGAA | | | |
| (positive control) | | | |
| anti- | Tfrc^{hum} | 0.02 | 0.01 |
| mTFRCscfv:hGAA | | | |
| (negative control) | | | |
| 12799scfv:hGAA | Tfrc^{hum} | 0.94 | 0.94 |
| 12839scfv:hGAA | Tfrc^{hum} | 0.49 | 0.62 |
| 12843scfv:hGAA | Tfrc^{hum} | 0.61 | 0.63 |
| 12847scfv:hGAA | Tfrc^{hum} | 1.90 | 1.33 |

Data were quantified from western blot as arbitrary units (FIG. 8). N = 1 per group.

### Example 2. Generation of Tfrc^{hum} GAA⁻ Animals

To explore the validity of the human TfR by modified mice generated in Example 1 as a mouse model to test anti-TfR binding protein based therapeutics, e.g., to test the ability of the human TfR expressed by these mice to transport macromolecules across the blood brain barrier, genetically modified embryonic stem cells comprising at least one allele comprising the Tfrc^{hum} genetic modification were further modified to collapse the GAA gene in these embryonic stem cells to create a Pompe Disease model.

Collapse of both Gaa alleles was achieved using a combination of four SpCas9 guide RNAs (gRNA), each consisting of invariant tracr RNA, the scaffold for binding to SpCas9 enzyme, and a 20 bp guide sequence specific to Gaa that allows a precise double-stranded cut. Guides direct SpCas9 cleavage close to the Gaa start ATG (guide 9251mGU (SEQ ID NO:29), cut site 38 bp upstream from the ATG; guide 9251mGU3 (SEQ ID NO:30), cut site 18 bp downstream of the ATG) and after the stop codon (guide 9251mGD3 (SEQ ID NO:31), cut site 677 bp downstream of the stop; guide 9251mGD4 (SEQ ID NO:32), cut site 705 bp downstream of the stop). See, e.g., FIGS. 4 and 5.

Specifically, a mixture of 125 pmol of each guide complexed with 31.25 pmol SpCas9 was electroporated into 2×10⁶ hybrid 12956/SvEvTac:C57BI/6NTac F1 embryonic stems cells (ESC) already containing humanization of one allele of mouse Tfrc. Resulting clonal colonies were screened first via TaqMan for deletion of both copies of Gaa, using loss-of-allele assays. See, Table 13.

TABLE 13

| Mouse TaqMan LOA | | | Mouse TaqMan Retention | | |
|---|---|---|---|---|---|
| 9251mTU | Fwd | CTCATGCTTCGGGAGTTAATGCT | 9251mretU | Fwd | GTGTGTCTGGTGACCCTGA |
| | | (SEQ ID NO: 33) | | | (SEQ ID NO: 36) |
| | Probe | CTCATGCTTCGGGAGTTAATGCT | | Probe | AACCGTCCTCTGTGCTGAGCCC |
| | (BHQ) | (SEQ ID NO: 34) | | (BHQ) | (SEQ ID NO: 37) |
| | Rev | GGTCGGTACGTCTTCCACAGT | | Rev | GTGGGCCTTCTGGAAATGG |
| | | (SEQ ID NO: 35) | | | (SEQ ID NO: 38) |
| 9251mTM | Fwd | GGGACCAACCTAGCTCACATCT | 9251mretD3 | Fwd | GCGATGGAGCCGTGGATT |
| | | (SEQ ID NO: 39) | | | (SEQ ID NO: 42) |
| | Probe | TGCCTCTTCCAGGACATGAACGA | | Probe | ACCTCAGGCTGTTACAGGGCTG |
| | (BHQ) | (SEQ ID NO: 40) | | (BHQ) | (SEQ ID NO: 43) |
| | Rev | CCCTGCTGAGAGCCTCTAAC | | Rev | CCCTCCCAGTTCAGGTCTCA |
| | | (SEQ ID NO: 41) | | | (SEQ ID NO: 44) |
| 9251mT | Fwd | GCCTGGCCATCCCTGTCT | | | |
| | | (SEQ ID NO: 45) | | | |
| | Probe | CACTGCTGATGGGAGAGCTGTTTCA | | | |
| | (BHQ) | (SEQ ID NO: 46) | | | |
| | Rev | GCCTCTGTAAACGACGGACTCT | | | |
| | | (SEQ ID NO: 47) | | | |

The deletion sizes were limited to within the region indicated by retention TaqMan assays. Clones with both Gaa copies deleted were then subjected to Illumina technology to fully characterize the knockout sequence using primers mm_Gaa_AmpF3 and mm_Gaa_AmpR1. See Table 14.

TABLE 14

| primer | Sequence (5′-3′) | Genome Build | location |
|---|---|---|---|
| mm_Gaa_AmpF3 | CCCACTCACCCTTTTCTCTTACAC | GRCm38/mm10 | chr11: 119,269,820- |
| | | | 119,269,843 (+) |
| mm_Gaa_AmpR1 | CCCGAAGCATGAGATGACCCAG | GRCm38/mm10 | chr11: 119,270,211- |
| | | | 119,270,232 (−) |

F0 mice were bred to homozygosity to generate Tfrc^{hum/hum} Gaa^{-/-} mice, also referred to herein as Tfrc^{hum}Gaa mice, Gaa^{-/-}/Tfrc^{hum} mice and the like.

### Example 3. Analysis of Tfrc^{hum} GAA⁻ Animals

Rescue of glycogen storage phenotype in Gaa^{-/-}/Tfrc^{hum} mice with AAV8 episomal liver depot anti-hTFRCscfv:GAA. Three anti-hTFRCscfv:GAA fusion proteins (12839, 12843, 12847) were tested in Pompe disease model to determine whether hTFRCscfv:GAA rescued the glycogen storage phenotype. All three normalized glycogen to Wt levels (FIGS. 9 and 10).

AAV production and in vivo transduction were performed as above. Gaa^{-/-}Tfrc^{hum} mice were dosed with 2e12 vg/kg AAV8. Tissues were harvested 4 weeks post-injection and flash-frozen as above. hGAA Western blot was performed as above (FIG. 11, Table 15).

Glycogen quantification (Table 16, FIG. 13). Tissues were dissected from mice immediately after sacrifice by CO₂ asphyxiation, snap frozen in liquid nitrogen, and stored at −80° C. Tissues were lysed on a benchtop homogenizer with stainless steel beads in distilled water for glycogen measurements or RIPA buffer for protein analyses. Glycogen analysis lysates were boiled and centrifuged to clear debris. Glycogen measurements were performed fluorometrically with a commercial kit according to manufacturer's instructions (K646, BioVision, Milpitas, CA, USA). The data show that three selected episomal AAV8 liver depot anti-hTfR antibody clones deliver mature GAA to the CNS, heart, and muscle in Gaa^{-/-}/Tfrc^{hum} mice.

TABLE 15
Quantification of hGAA protein in tissues of Gaa^{-/-}
/Tfrc^{hum} mice treated with liver-depot AA V8 anti-hTFRCscfv:hGAA.

| Treatment | | | | | | Spinal | | |
|---|---|---|---|---|---|---|---|---|
| group | n | Serum* | Liver* | Cerebrum** | Cerebellum** | Cord*** | Heart** | Quadricep** |
| Gaa^{-/-} Untreated | 1 | 0.00 | 0.02 | 0.00 | 0.00 | 0.00 | 0.02 | 0.01 |
| Gaa^{-/-} | 3 | 2.42 ± | 1.63 ± | 0.14 ± | 0.13 ± | 0.19 ± | 0.53 ± | 0.14 ± |
| 12839scfv:hGAA | | 2.41 | 0.96 | 0.12 | 0.12 | 0.19 | 0.52 | 0.16 |
| Gaa^{-/-} | 3 | 2.07 ± | 2.23 ± | 0.17 ± | 0.11 ± | 0.17 ± | 0.49 ± | 0.18 ± |
| 12843scfv:hGAA | | 1.35 | 0.08 | 0.07 | 0.05 | 0.09 | 0.31 | 0.06 |
| Gaa^{-/-} | 3 | 1.56 ± | 1.40 ± | 0.25 ± | 0.21 ± | 0.42 ± | 0.58 ± | 0.19 ± |
| 12847scfv:hGAA | | 0.71 | 0.13 | 0.04 | 0.09 | 0.19 | 0.17 | 0.08 |

Data were quantified from western blot as arbitrary units (FIG. 4).
All values are mean ± SD, n = 1-3 per group.
*Total hGAA protein;
**Mature hGAA protein.

TABLE 16
Quantification of glycogen in tissues of Gaa^{-/-}/Tfr^{chum}
mice treated with liver-depot AAV8 anti-hTFRCscfv:hGAA.

| | | | Spinal | | |
|---|---|---|---|---|---|
| Treatment group | Cerebrum | Cerebellum | Cord | Heart | Quadricep |
| Wt Untreated | 0.06 ± 0.04* | 0.01 ± 0.04* | 0.05 ± 0.05* | 0.08 ± 0.02* | 0.34 ± 0.19* |
| Gaa−/− Untreated | 2.34 ± 0.58 | 2.51 ± 0.38 | 3.08 ± 0.23 | 25.30 ± 6.06 | 13.05 ± 0.98 |
| Gaa−/− | 0.11 ± 0.03* | 0.46 ± 0.08* | 0.08 ± 0.10* | 0.68 ± 0.68* | 2.15 ± 2.52* |
| 12839scfv:hGAA | | | | | |
| Gaa−/− | 0.09 ± 0.02* | 0.09 ± 0.08* | 0.13 ± 0.13* | 0.09 ± 0.01* | 1.22 ± 1.39* |
| 12843scfv:hGAA | | | | | |
| Gaa−/− | 0.05 ± 0.01* | 0.02 ± 0.03* | 0.20 ± 0.33* | 0.11 ± 0.11* | 0.80 ± 0.79* |
| 12847scfv:hGAA | | | | | |

All values are glycogen μg/mg tissue, mean ± SD, n = 3-4 per group.
One Way ANOVA
*p < 0.0001 vs. Gaa^{-/-} Untreated group.

Rescue of glycogen storage in brain and muscle in Gaa^{-/-}/Tfrc^{hum} mice with AAV8 episomal liver depot anti-hTFRCscfv:GAA. Three selected anti-hTFRCscfv:GAA fusion proteins (12799, 12843, and 12847) were tested in Pompe disease model mice to determine whether hTFRCscfv:GAA rescued the glycogen storage phenotype. In this experiment, histology on brain and muscle sections was performed to visualize glycogen in the tissues. All three selected anti-hTFRCscfv:GAA proteins reduced glycogen staining in the brain and muscle.

AAV production and in vivo transduction were performed as above. Three-month old Gaa^{-/-}/Tfrc^{hum} mice were dosed with 4e11 vg/kg AAV8. 4 weeks post-injection, tissues were frozen for glycogen analysis as above (Table 17). For histology, animals were perfused with saline (0.9% NaCl), and tissues were drop-fixed overnight in 10% Normal Buffered Formalin. Tissues were washed 3× in PBS and stored in PBS/0.01% sodium azide until embedding. Tissues were embedded in paraffin and 5 um sections were cut from brain (coronal, −2 mm bregma) and quadricep (fiber cross-section). Sections were stained with Periodic Acid-Schiff and Hematoxylin using standard protocols (FIGS. 11A-11D).

TABLE 17
Quantification of glycogen in tissues of Gaa^{-/-}
/Tfrc^{hum} mice treated with liver-depot AAV8 anti-hTFRCscfv:hGAA.

| Treatment group | Cerebellum | Quadricep |
|---|---|---|
| Wt Untreated | 0.02 ± 0.03* | 0.55 ± 0.10* |
| Gaa−/− Untreated | 1.91 ± 0.26 | 12.19 ± 3.02 |
| Gaa−/− 12799scfv:hGAA | 0.10 ± 0.06* | 1.34 ± 0.9* |
| Gaa−/− 12843scfv:hGAA | 0.09 ± 0.06* | 1.09 ± 1.27* |
| Gaa−/− 12847scfv:hGAA | 0.07 ± 0.06* | 0.72 ± 0.64* |

All values are glycogen μg/mg tissue, mean ± SD, n = 5-8 per group.
One Way ANOVA
*p < 0.0001 vs. Gaa^{-/-} Untreated group.

Insertion of Anti-hTFRC 12847Scfv:GAA in Albumin Locus of Gaa^{-/-}/Tfrc^{hum} Mice

Anti-hTFRC 12847scfv:GAA was further tested in Pompe disease model mice as described herein by albumin insertion to determine whether the results seen with episomal AAV8 liver depot expression could be replicated. Albumin insertion of 12847scfv:GAA delivered mature hGAA protein to the brain and muscle, and rescued the glycogen storage phenotype in Gaa^{-/-}Tfrc^{hum} mice.

AAV production: A promoterless AAV genome plasmid was created with the 12847scfv:GAA sequence and the mouse albumin exon 1 splice acceptor site at the 3′ end. Recombinant AAV8 (AAV2/8) was produced in HEK293 cells. Cells were transfected with three plasmids encoding adenovirus helper genes, AAV8 rep and cap genes, and recombinant AAV genomes containing transgenes flanked by AAV2 inverted terminal repeats (ITRs). On day 5, cells and medium were collected, centrifuged, and processed for AAV purification. Cell pellets were lysed by freeze-thaw and cleared by centrifugation. Processed cell lysates and medium were overlaid onto iodixanol gradients columns and centrifuged in an ultracentrifuge. Virus fractions were removed from the interface between the 40% and 60% iodixanol solutions and exchanged into 1×PBS with desalting columns. AAV vg were quantified by ddPCR.

In vivo CRISPR/Cas9 insertion into the albumin locus: 3 month old Gaa^{-/-}/Tfrc^{hum} mice were dosed via tail vein injection with 3e12 vg/kg AAV8 12847scfv:GAA and 3 mg/kg LNP gRNA/Cas9 mRNA diluted in PBS+0.001% F-68 Pluronic. Mice were sacrificed 3 weeks post injection. Negative control mice received insertion AAV8 without LNP. Positive control mice were dosed with 4e11 vg/kg episomal liver depot AAV8 12847scfv:GAA under the TTR promoter (phenotype rescue data previously shown). Tissues were dissected from mice immediately after sacrifice by CO2 asphyxiation, snap frozen in liquid nitrogen, and stored at −80° C. Blood was collected from mice by cardiac puncture immediately following CO2 asphyxiation and serum was separated using serum separator tubes (BD Biosciences, 365967).

TABLE 18
Treatment groups and controls:

| Treatment group | Genotype | Function |
|---|---|---|
| Wt Untreated | Tfrc^{hum} | Normal untreated mouse control |
| Gaa^{-/-} untreated | Gaa^{-/-}/Tfrc^{hum} | Untreated Pompe disease mouse |
| Gaa^{-/-} insertion | Gaa^{-/-}/Tfrc^{hum} | Negative control for insertion (no Cas9/gRNA |
| AAV only | | delivered) |
| Gaa^{-/-} episomal | Gaa^{-/-}/Tfrc^{hum} | Positive control, previously shown rescue of |
| AAV8 TTR | | glycogen storage phenotype |
| 12847scfv:hGAA | | |
| Gaa^{-/-} insertion | Gaa^{-/-}/Tfrc^{hum} | |
| 12847scfv:hGAA | | Experimental insertion group |
| Gaa^{-/-} untreated | Gaa^{-/-}/ | Untreated Pompe disease mouse (CD63 |
| | Cd63^{hum} | humanized) |
| Gaa^{-/-} insertion | Gaa^{-/-}/ | Negative control for BBB-crossing (muscle |
| anti- | Cd63^{hum} | targeted) |
| CD63scfv:hGAA | | |

### Western Blot: (Table 19, FIG. 12A)

Tissue lysates were prepared by lysis in RIPA buffer with protease inhibitors (1861282, Thermo Fisher, Waltham, MA, USA).Tissue lysates were homogenized with a bead homogenizer (FastPrep5, MP Biomedicals, Santa Ana, CA, USA). Cells or tissue lysates were run on SDS-PAGE gels using the Novex system (LifeTech).

Thermo, XPO4200BOX, LC2675, LC3675, LC2676). Gels were transferred to low-fluorescence polyvinylidene fluoride (PVDF) membrane (IPFL07810, LI-COR, Lincoln, NE, USA) and stained with Revert 700 Total Protein Stain (TPS; 926-11010 LI-COR, Lincoln, NE, USA), followed by blocking with Odyssey blocking buffer (927-500000, LI-COR, Lincoln, NE, USA) in Tris buffer saline with 0.1% Tween 20 and staining with antibodies against GAA (ab137068, Abcam, Cambridge, MA, USA), or anti-GAPDH (ab9484, Abcam, Cambridge, MA, USA) and the appropriate secondary (926-32213 or 925-68070, LI-COR, Lincoln, NE, USA). Blots were imaged with a LI-COR Odyssey CLx.

Protein band intensity was quantified in LI-COR Image Studio software. The quantification of the mature 77 kDa GAA band for each sample was determined by normalizing to the lane's TPS signal (loading control).

### Glycogen Quantification: (Table 19, FIG. 12B)

Tissues were dissected from mice immediately after sacrifice by CO₂ asphyxiation, snap frozen in liquid nitrogen, and stored at −80° C. Tissues were lysed on a benchtop homogenizer with stainless steel beads in distilled water for glycogen measurements or RIPA buffer for protein analyses. Glycogen analysis lysates were boiled and centrifuged to clear debris. Glycogen measurements were performed fluorometrically with a commercial kit according to manufacturer's instructions (K646, BioVision, Milpitas, CA, USA).

TABLE 19
Quantification of hGAA protein in tissues of Gaa^{-/-}
/Tfrc^{hum} mice treated with insertion anti-hTFRC 12847scfv:hGAA

| | | | Cerebrum | Quadricep |
|---|---|---|---|---|
| | Liver | Serum | mature | mature |
| Treatment group | total hGAA | total hGAA | hGAA | hGAA |
| Gaa^{-/-} insertion | 0.02 ± 0.003 | 0.03 ± 0.02 | 0.002 ± 0.001 | 0.006 ± 0.002 |
| AAV only negative | | | | |
| control | | | | |
| Gaa^{-/-} episomal | 2.35 ± 0.72 | 3.65 ± 2.09 | 0.49 ± 0.20§§ | 0.148 ± 0.043§§ |
| AAV8 TTR | | | | |
| 12847scfv:hGAA | | | | |
| Gaa^{-/-} insertion | 4.31 ± 0.87* | 3.47 ± 2.37 | 0.57 ± 0.26§§ | 0.141 ± 0.062§§ |
| 12847scfv:hGAA | | | | |
| Gaa^{-/-} insertion | 2.67 ± 1.04* | 0.93 ± 0.55* | 0.01 ± 0.003 | 0.060 ± 0.037 |
| anti- | | | | |
| CD63scfv:hGAA | | | | |

All values are arbitrary units, mean ± SD, n = 3-8 per group.
One Way ANOVA
*p < 0.05 vs. Gaa^{-/-} episomal AAV8 TTR 12847scfv:GAA group;
^{§§}p < 0.001 vs. AAV only negative control group.

TABLE 20
Quantification of glycogen in tissues of Gaa^{-/-}
/Tfrc^{hum} mice treated with insertion anti-hTFRC 12847scfv:hGAA

| Treatment group | Cerebrum | Quadricep |
|---|---|---|
| Wt untreated | 0.10 ± 0.07 | 0.37 ± 0.13 |
| Gaa^{-/-}/Tfrc^{hum} untreated (Tfrc^{hum}) | 2.76 ± 0.41 | 12.75 ± 1.88 |
| Gaa^{-/-}/Tfrc^{hum} insertion AAV only | 2.17 ± 0.40* | 10.64 ± 2.56 |
| Gaa^{-/-}/Tfrc^{hum} episomal AAV8 TTR | 0.13 ± 0.03***^{§} | 2.44 ± 2.21***^{§} |
| 12847scfv:hGAA | | |
| Gaa^{-/-}/Tfrc^{hum} insertion 12847scfv:hGAA | 0.16 ± 0.05***^{§} | 1.67 ± 0.76***^{§} |
| Gaa^{-/-}/Cd63^{hum} untreated | 2.34 ± 0.30 | 11.91 ± 1.01 |
| Gaa^{-/-}/Cd63^{hum} insertion anti-CD63scfv:hGAA | 1.71 ± 0.20* | 4.06 ± 0.13** |

All values are glycogen ug/mg tissue, mean ± SD, n = 3-8 per group.
One Way ANOVA
*p < 0.01 vs. Gaa^{-/-}/Cd63^{hum} untreated group;
**p < 0.001 vs. Gaa^{-/-}/Cd63^{hum} untreated group;
***p < 0.0001 vs. Gaa^{-/-}/Tfrc^{hum} untreated group;
^{§}non-significant vs. Wt untreated group.

In summary, the anti-TfR:GAA protein expressed from hepatocytes are successfully delivered to muscle cells and CNS cells in the tested mice, demonstrating efficient intracellularization and transport across the blood brain barrier in these mice by the human TfR protein expressed therein.

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants to relate to each and every individual publication, database entry (e.g., Genbank sequences or GeneID entries), patent application, or patent, each of which is clearly identified in even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

1. A non-human animal cell comprising a nucleic acid sequence encoding a heterologous Transferrin Receptor (TfR) protein or a portion thereof.

2-22. (canceled)

23. A non-human animal comprising a nucleic acid sequence encoding a heterologous transferrin receptor (TfR) protein or a portion thereof.

24. The non-human animal of claim 23, wherein the nucleic acid sequence encoding the heterologous TfR protein or the portion thereof comprises:
(i) a nucleic acid sequence comprising exon 2 of a human TFRC gene or a coding portion thereof,
(ii) a nucleic acid sequence comprising exon 3 of a human TFRC gene or a portion thereof,
(iii) a nucleic acid sequence comprising exon 4 of a human TFRC gene or a portion thereof,
(iv) a nucleic acid sequence comprising exon 5 of a human TFRC gene or a portion thereof,
(v) a nucleic acid sequence comprising exon 6 of a human TFRC gene or a portion thereof,
(vi) a nucleic acid sequence comprising exon 7 of a human TFRC gene or a portion thereof,
(vii) a nucleic acid sequence comprising exon 8 of a human TFRC gene or a portion thereof,
(viii) a nucleic acid sequence comprising exon 9 of a human TFRC gene or a portion thereof,
(ix) a nucleic acid sequence comprising exon 10 of a human TFRC gene or a portion thereof,
(x) a nucleic acid sequence comprising exon 11 of a human TFRC gene or a portion thereof,
(xi) a nucleic acid sequence comprising exon 12 of a human TFRC gene or a portion thereof,
(xii) a nucleic acid sequence comprising exon 13 of a human TFRC gene or a portion thereof,
(xiii) a nucleic acid sequence comprising exon 14 of a human TFRC gene or a portion thereof,
(xiv) a nucleic acid sequence comprising exon 15 of a human TFRC gene or a portion thereof,
(xv) a nucleic acid sequence comprising exon 16 of a human TFRC gene or a portion thereof,
(xvi) a nucleic acid sequence comprising exon 17 of a human TFRC gene or a portion thereof,
(xvii) a nucleic acid sequence comprising exon 18 of a human TFRC gene or a portion thereof,
(xviii) a nucleic acid sequence comprising exon 19 of a human TFRC gene or a coding portion thereof, or
(xix) any combination of (i)-(xviii).

25. The non-human animal of claim 23, wherein the nucleic acid sequence encoding the heterologous TfR protein or the portion thereof comprises:
(i) a nucleic acid sequence comprising exon 2 of a human TFRC gene or a coding portion thereof and intron 2 of a human TFRC gene or a portion thereof,
(ii) a nucleic acid sequence comprising exon 3 of a human TFRC gene or a portion thereof and intron 3 of a human TFRC gene or a portion thereof,
(iii) a nucleic acid sequence comprising exon 4 of a human TFRC gene or a portion thereof and intron 4 of a human TFRC gene or a portion thereof,
(iv) a nucleic acid sequence comprising exon 5 of a human TFRC gene or a portion thereof and intron 5 of a human TFRC gene or a portion thereof,
(v) a nucleic acid sequence comprising exon 6 of a human TFRC gene or a portion thereof and intron 6 of a human TFRC gene or a portion thereof,
(vi) a nucleic acid sequence comprising exon 7 of a human TFRC gene or a portion thereof and intron 7 of a human TFRC gene or a portion thereof,
(vii) a nucleic acid sequence comprising exon 8 of a human TFRC gene or a portion thereof and intron 8 of a human TFRC gene or a portion thereof,
(viii) a nucleic acid sequence comprising exon 9 of a human TFRC gene or a portion thereof and intron 9 of a human TFRC gene or a portion thereof,
(ix) a nucleic acid sequence comprising exon 10 of a human TFRC gene or a portion thereof and intron 10 of a human TFRC gene or a portion thereof,
(x) a nucleic acid sequence comprising exon 11 of a human TFRC gene or a portion thereof and intron 11 of a human TFRC gene or a portion thereof,
(xi) a nucleic acid sequence comprising exon 12 of a human TFRC gene or a portion thereof and intron 12 of a human TFRC gene or a portion thereof,
(xii) a nucleic acid sequence comprising exon 13 of a human TFRC gene or a portion thereof and intron 13 of a human TFRC gene or a portion thereof,
(xiii) a nucleic acid sequence comprising exon 14 of a human TFRC gene or a portion thereof and intron 14 of a human TFRC gene or a portion thereof,
(xiv) a nucleic acid sequence comprising exon 15 of a human TFRC gene or a portion thereof and intron 15 of a human TFRC gene or a portion thereof,
(xv) a nucleic acid sequence comprising exon 16 of a human TFRC gene or a portion thereof and intron 16 of a human TFRC gene or a portion thereof,
(xvi) a nucleic acid sequence comprising exon 17 of a human TFRC gene or a portion thereof and intron 17 of a human TFRC gene or a portion thereof,
(xvii) a nucleic acid sequence comprising exon 18 of a human TFRC gene or a portion thereof and intron 18 of a human TFRC gene or a portion thereof,
(xviii) a nucleic acid sequence comprising exon 19 of a human TFRC gene or a coding portion thereof, or
(xix) any combination of (i)-(xviii).

26. The non-human animal of claim 23, wherein the nucleic acid sequence encoding the heterologous TfR protein or the portion thereof comprises a nucleic acid sequence selected from the group consisting of a nucleic acid sequence set forth as SEQ ID NO:5, a nucleic acid sequence set forth as SEQ ID NO:6, a nucleic acid sequence set forth as SEQ ID NO:9, and a nucleic acid sequence set forth as SEQ ID NO:10.

27. The non-human animal of claim 23, wherein the nucleic acid sequence encoding the heterologous TfR protein or portion thereof is at an endogenous Tfrc locus.

28. The non-human animal of claim 23, wherein the nucleic acid sequence encoding the heterologous TfR protein or portion thereof replaces an orthologous endogenous nucleic acid sequence encoding an endogenous TfR protein or a portion thereof.

29. The non-human animal of claim 23, wherein the non-human animal, comprises an endogenous Tfrc locus, and wherein the endogenous Tfrc locus comprises an endogenous Tfrc gene comprising a heterozygous or homozygous replacement of an endogenous nucleic acid sequence encoding an endogenous TfR protein or a portion thereof with the nucleic acid sequence encoding the heterologous TfR protein or the portion thereof,
wherein the endogenous nucleic acid sequence encoding the endogenous TfR protein or the portion thereof and the nucleic acid sequence encoding the heterologous TfR protein or the portion thereof are orthologous.

30. The non-human animal of claim 23, wherein the heterologous TfR protein or the portion thereof comprises an amino acid sequence of a human TfR protein or a portion thereof.

31. The non-human animal of claim 23, wherein the heterologous TfR protein or the portion thereof comprises:
(i) an amino acid sequence set forth as SEQ ID NO:4;
(ii) an amino acid sequence set forth as SEQ ID NO:25;
(iii) an amino acid sequence set forth as SEQ ID NO:26;
(iv) an amino acid sequence set forth as SEQ ID NO:27;
(v) an amino acid sequence set forth as SEQ ID NO:28; or
(vi) any combination of (i)-(v).

32. The non-human animal of claim 23, wherein the heterologous TfR protein or the portion thereof comprises an amino acid sequence set forth as SEQ ID NO:25.

33. The non-human animal of claim 23, wherein the non-human animal is a mammal.

34. The non-human animal of claim 23, wherein the non-human animal is a rodent.

35. The non-human animal of claim 23, wherein the non-human animal is rat or a mouse.

36. The non-human animal of claim 23, wherein the non-human animal comprises a non-human animal cell identified in Table 1 that expresses the heterologous TfR protein or portion thereof on the cell surface.

37. The non-human animal of claim 23, wherein the non-human animal comprises a non-human animal blood brain barrier endothelial cell that expresses the heterologous TfR protein or portion thereof on the cell surface.

38. The non-human animal of claim 23, wherein the heterologous TfR protein comprises a full-length human TfR protein, and
wherein the non-human animal comprises a non-human animal cell identified in Table 1 that expresses the full-length human TfR protein on the cell surface.

39. The non-human animal of claim 23, wherein the non-human animal comprises a non-human animal cell that does not express the heterologous TfR protein or portion thereof on the cell surface.

40. The non-human animal of claim 23, wherein the non-human animal comprises a non-human animal germ cell that does not express the heterologous TfR protein or the portion thereof.

41. The non-human animal of claim 23, wherein the non-human animal is a mouse.

42. A non-human animal genome, wherein the non-human animal genome comprises a nucleic acid sequence encoding a heterologous transferrin receptor (TfR) protein or a portion thereof.

43-55. (canceled)

56. A chimeric nucleic acid molecule, comprising a nucleic acid sequence of a modified non-human animal Tfrc gene that (a) encodes a TfR protein and (b) comprises a replacement of a sequence encoding the TfR protein or portion thereof with a homologous sequence encoding a heterologous TfR protein or a portion thereof,
wherein the chimeric nucleic acid molecule encodes a functional TfR protein.

57-63. (canceled)

64. A method of making the non-human animal cell of claim 1, comprising inserting the nucleic acid sequence encoding the heterologous TfR protein or the portion thereof into the genome of the non-human animal cell.

65-95. (canceled)

96. A non-human animal comprising a knockout mutation of an endogenous α-glucosidase (Gaa) gene.

97-115. (canceled)

116. A non-human animal model for testing an anti-human-TfR binding protein, comprising:
(i) a non-human animal comprising a nucleic acid sequence encoding a heterologous transferrin receptor (TfR) protein or a portion thereof, or
(ii) a non-human animal comprising a knockout mutation of an endogenous α-glucosidase (Gaa) gene and a nucleic acid sequence encoding a heterologous TfR protein or a portion thereof; and
iii an anti-human-TfR binding protein that binds human TfR.

117-120. (canceled)


SEQUENCE LISTING (52 sequences)

SEQ ID NO: 1 - DNA, 2292 bp, Mus musculus
  source 1..2292: mol_type = other DNA
atgatggatcaagccagatcagcattctctaacttgtttggtggggaaccattgtcatacacccggtttagccttgctcggcaagtagatggagataacagtcatgtggagatgaaactggctgcagatgaagaagaaaatgccgacaataacatgaaggctagtgtcagaaaacccaagaggtttaatggaagactctgctttgcagctattgcactagtcattttcttcttgattggattcatgagtggctacctgggctattgtaagcgtgtagaacaaaaagaggagtgtgtgaaactggctgaaacggaggagacagacaagtcagaaaccatggaaacagaggatgttcctacatcatctcgcttatattgggcagacctcaaaacactgttgtcagagaagttgaactccatagagtttgctgacaccatcaagcagctgagccagaatacatacactcctcgtgaggctggatctcaaaaagatgaaagtcttgcctattatattgaaaatcagttccatgaatttaaattcagcaaagtctggcgagatgaacactatgtgaagattcaagtgaaaagcagcattggtcaaaacatggtgaccatagtgcagtcaaatggtaacttagacccagtggagtctcccgagggttatgtggcattcagtaaacctacagaagtttctggtaaactggtccatgctaattttggcactaaaaaggactttgaagaactaagttattctgtgaatggatctttagtgattgttagagcaggggaaattacttttgcagaaaaggttgcaaatgcccaaagctttaatgcaattggtgtcctcatatacatggacaagaataaattccccgttgttgaggcagaccttgcactctttggacatgctcatctaggaactggtgatccatacacacctggctttccttctttcaatcatactcagtttccgccatctcagtcatcagggttgcctaatatacctgtgcaaacaatctcaagagctgctgcagaaaagctatttggaaaaatggaaggaagctgtcctgctagatggaacatagattcttcatgtaagctggaactttcacagaatcaaaatgtgaagctcattgtgaaaaacgtactgaaagaaagaagaatacttaacatctttggagttattaaaggttatgaggaaccagaccgttatgttgtagtaggagcccagagagacgctttgggtgctggtgttgcggcgaagtccagtgtgggaacaggtcttctgttgaaacttgcccaagtattctcagatatgatttcaaaagatggatttagacccagcagaagtataatctttgccagctggactgcaggcgactttggagctgttggtgccactgagtggttggagggatacctttcatctttgcatttaaaagctttcacttatattaatttggataaagttgtccttggtactagtaacttcaaagtttctgccagccccttattatatacacttatgggaaagataatgcaagatgtaaagcatccagttgatggaaaatctctatatagagacagcaattggattagcaaagttgagaaactttcctttgacaatgctgcatatcctttccttgcatattctggaatcccagcagtttctttttgtttttgtgaggatgcagactatccttatttgggcactagattggatacctatgaggcattgactcagaaagttcctcagctcaaccaaatggttcgtacagcagcggaagtggctggtcagctcattattaaacttacccatgacgttgaattgaacctggactatgagatgtataacagcaaactactgtcatttatgaaggatctgaaccagttcaaaacagatatcagggatatgggtctaagtctacagtggctgtattccgctcgtggagactacttccgtgctacttctagactaacaactgattttcataatgctgagaaaacaaacagatttgtcatgagggaaatcaatgatcgtattatgaaagtggagtatcacttcctgtcgccctatgtatctccaagagagtctcctttccgacatatcttctggggctctggctctcacactctctcagctttagtggagaacttgaagcttcgtcaaaaaaatattactgcttttaatgaaaccctcttcagaaaccagttggccctggctacttggactattcagggagtcgcaaatgccctctctggtgacatttggaatattgacaatgagttttaa

SEQ ID NO: 2 - protein, 763 aa, Mus musculus
MMDQARSAFSNLFGGEPLSYTRFSLARQVDGDNSHVEMKLAADEEENADNNMKASVRKPKRFNGRLCFAAIALVIFFLIGFMSGYLGYCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 3 - DNA, 2283 bp, Homo sapiens
  source 1..2283: mol_type = other DNA
atgatggatcaagctagatcagcattctctaacttgtttggtggagaaccattgtcatatacccggttcagcctggctcggcaagtagatggcgataacagtcatgtggagatgaaacttgctgtagatgaagaagaaaatgctgacaataacacaaaggccaatgtcacaaaaccaaaaaggtgtagtggaagtatctgctatgggactattgctgtgatcgtctttttcttgattggatttatgattggctacttgggctattgtaaaggggtagaaccaaaaactgagtgtgagagactggcaggaaccgagtctccagtgagggaggagccaggagaggacttccctgcagcacgtcgcttatattgggatgacctgaagagaaagttgtcggagaaactggacagcacagacttcaccggcaccatcaagctgctgaatgaaaattcatatgtccctcgtgaggctggatctcaaaaagatgaaaatcttgcgttgtatgttgaaaatcaatttcgtgaatttaaactcagcaaagtctggcgtgatcaacattttgttaagattcaggtcaaagacagcgctcaaaactcggtgatcatagttgataagaacggtagacttgtttacctggtggagaatcctgggggttatgtggcgtatagtaaggctgcaacagttactggtaaactggtccatgctaattttggtactaaaaaagattttgaggatttatacactcctgtgaatggatctatagtgattgtcagagcagggaaaatcacctttgcagaaaaggttgcaaatgctgaaagcttaaatgcaattggtgtgttgatatacatggaccagactaaatttcccattgttaacgcagaactttcattctttggacatgctcatctggggacaggtgacccttacacacctggattcccttccttcaatcacactcagtttccaccatctcggtcatcaggattgcctaatatacctgtccagacaatctccagagctgctgcagaaaagctgtttgggaatatggaaggagactgtccctctgactggaaaacagactctacatgtaggatggtaacctcagaaagcaagaatgtgaagctcactgtgagcaatgtgctgaaagagataaaaattcttaacatctttggagttattaaaggctttgtagaaccagatcactatgttgtagttggggcccagagagatgcatggggccctggagctgcaaaatccggtgtaggcacagctctcctattgaaacttgcccagatgttctcagatatggtcttaaaagatgggtttcagcccagcagaagcattatctttgccagttggagtgctggagactttggatcggttggtgccactgaatggctagagggatacctttcgtccctgcatttaaaggctttcacttatattaatctggataaagcggttcttggtaccagcaacttcaaggtttctgccagcccactgttgtatacgcttattgagaaaacaatgcaaaatgtgaagcatccggttactgggcaatttctatatcaggacagcaactgggccagcaaagttgagaaactcactttagacaatgctgctttccctttccttgcatattctggaatcccagcagtttctttctgtttttgcgaggacacagattatccttatttgggtaccaccatggacacctataaggaactgattgagaggattcctgagttgaacaaagtggcacgagcagctgcagaggtcgctggtcagttcgtgattaaactaacccatgatgttgaattgaacctggactatgagaggtacaacagccaactgctttcatttgtgagggatctgaaccaatacagagcagacataaaggaaatgggcctgagtttacagtggctgtattctgctcgtggagacttcttccgtgctacttccagactaacaacagatttcgggaatgctgagaaaacagacagatttgtcatgaagaaactcaatgatcgtgtcatgagagtggagtatcacttcctctctccctacgtatctccaaaagagtctcctttccgacatgtcttctggggctccggctctcacacgctgccagctttactggagaacttgaaactgcgtaaacaaaataacggtgcttttaatgaaacgctgttcagaaaccagttggctctagctacttggactattcagggagctgcaaatgccctctctggtgacgtttgggacattgacaatgagttttaa

SEQ ID NO: 4 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 5 - DNA, 2283 bp, synthetic construct
  source 1..2283: mol_type = other DNA
atgatggatcaagccagatcagcattctctaacttgtttggtggagaaccattgtcatatacccggttcagcctggctcggcaagtagatggcgataacagtcatgtggagatgaaacttgctgtagatgaagaagaaaatgctgacaataacacaaaggccaatgtcacaaaaccaaaaaggtgtagtggaagtatctgctatgggactattgctgtgatcgtctttttcttgattggatttatgattggctacttgggctattgtaaaggggtagaaccaaaaactgagtgtgagagactggcaggaaccgagtctccagtgagggaggagccaggagaggacttccctgcagcacgtcgcttatattgggatgacctgaagagaaagttgtcggagaaactggacagcacagacttcaccggcaccatcaagctgctgaatgaaaattcatatgtccctcgtgaggctggatctcaaaaagatgaaaatcttgcgttgtatgttgaaaatcaatttcgtgaatttaaactcagcaaagtctggcgtgatcaacattttgttaagattcaggtcaaagacagcgctcaaaactcggtgatcatagttgataagaacggtagacttgtttacctggtggagaatcctgggggttatgtggcgtatagtaaggctgcaacagttactggtaaactggtccatgctaattttggtactaaaaaagattttgaggatttatacactcctgtgaatggatctatagtgattgtcagagcagggaaaatcacctttgcagaaaaggttgcaaatgctgaaagcttaaatgcaattggtgtgttgatatacatggaccagactaaatttcccattgttaacgcagaactttcattctttggacatgctcatctggggacaggtgacccttacacacctggattcccttccttcaatcacactcagtttccaccatctcggtcatcaggattgcctaatatacctgtccagacaatctccagagctgctgcagaaaagctgtttgggaatatggaaggagactgtccctctgactggaaaacagactctacatgtaggatggtaacctcagaaagcaagaatgtgaagctcactgtgagcaatgtgctgaaagagataaaaattcttaacatctttggagttattaaaggctttgtagaaccagatcactatgttgtagttggggcccagagagatgcatggggccctggagctgcaaaatccggtgtaggcacagctctcctattgaaacttgcccagatgttctcagatatggtcttaaaagatgggtttcagcccagcagaagcattatctttgccagttggagtgctggagactttggatcggttggtgccactgaatggctagagggatacctttcgtccctgcatttaaaggctttcacttatattaatctggataaagcggttcttggtaccagcaacttcaaggtttctgccagcccactgttgtatacgcttattgagaaaacaatgcaaaatgtgaagcatccggttactgggcaatttctatatcaggacagcaactgggccagcaaagttgagaaactcactttagacaatgctgctttccctttccttgcatattctggaatcccagcagtttctttctgtttttgcgaggacacagattatccttatttgggtaccaccatggacacctataaggaactgattgagaggattcctgagttgaacaaagtggcacgagcagctgcagaggtcgctggtcagttcgtgattaaactaacccatgatgttgaattgaacctggactatgagaggtacaacagccaactgctttcatttgtgagggatctgaaccaatacagagcagacataaaggaaatgggcctgagtttacagtggctgtattctgctcgtggagacttcttccgtgctacttccagactaacaacagatttcgggaatgctgagaaaacagacagatttgtcatgaagaaactcaatgatcgtgtcatgagagtggagtatcacttcctctctccctacgtatctccaaaagagtctcctttccgacatgtcttctggggctccggctctcacacgctgccagctttactggagaacttgaaactgcgtaaacaaaataacggtgcttttaatgaaacgctgttcagaaaccagttggctctagctacttggactattcagggagctgcaaatgccctctctggtgacgtttgggacattgacaatgagttttaa

SEQ ID NO: 6 - DNA, 2283 bp, synthetic construct; identical to SEQ ID NO: 5
  source 1..2283: mol_type = other DNA

SEQ ID NO: 7 - DNA, 5320 bp, Mus musculus
  source 1..5320: mol_type = other DNA
cagagggccggccggggggtggggccgagctataagctttgggtgggaggcagcgctgccttcagaaggcgtgcggagcgcgggctgctgcattgcggactgtagaggcgcttcctagtgagtgactcccttgtcagcggcacggcccatcgtggtcctcgcgtggcgggcggaccagagcgagacgccagggcctgggtggtgcgggcggggaggcggaggggtgtcgcggagtccggggctgaggagcgcgggttgcaggtgcagcgcggtgggtgtggggagccgctgtaccctgcgcccctcgggtcctccgggccttcgcaggccagtgctaggccgcgggttcgagagtcaccacgctgaggcgcaggcttgttccgccgggagcacgtggtggcggctggaggaagtcgccccagggaacggctgtcggggtacgtgggtgaccttggggcccctcgcaggagggcgtcacagctgaaaaggacaaagctgttttctattcggttactagtgtcacggacatttagaggggcggggggagcttccaataactgcacgttggaacttcggcaccacctggtcggtttttttgccagtctctccctcttggcccagcgtgtggaatctcatttttctagggcagaataggtctgaacgctgcaggtaatactaagaacgtctctagcatctcctaagatgggagaacgtagaaatacgacctctttgtacgagctcttttagaactagctgtagagaaccagcgtgcaccctggtgttggacagctctctaaaatggtgtttgagggtaagaaaactgcatttgcaaatttttcagttagcacactttgtcccgagcgtaaaatgaaatgatctccttatacttaggcagaagactggactggattgctgttcagtttctgtgctatttttttaaaataggatttaagtgggaaatagttgtgttacagaaattctcggctattctgtgctatttttttttaaataggatttaagtgggaaatagttgtgttacagaaattctcggctacctgatacttttattctaagattagatgagttggctctgagctgtgaaatatgacctctttgacaaagacatttaagctgattcaggatgttatctacaaagaaaacgggatttagcttgtgtgggtccacttgcatttattttctgttaaggattataataaactgctttatacaggaatccaatatcagctgttttttatatagagagcataatactttttactttgagagaggatgttgtcaaggaatggtggctgtgaacctggcctgtgttgactggttagatctgtctgcctaaccccaccctaaggctaagtagttatatgcttgtggcaatgtgcttatttataatagggcaagattatgggctaaatttgggttagacaacaatgaaagttaattaaacgaccctcaggccttgggtctactatgtgtaagtgatttccttctctcccagatgagtgctatacaaaataaacttcagtgacctcaagtggttttgaccttttggttgctattcagaaaactatggaaatgaaaacctgctacctatttcctattgccttttcaatttcccaaagaaggtctcccctatgaatccatgggtagccttgaactcagatccacctgcttctgtttggagagtgataagattaaaggcaagtgccaccacacccagcaaagtaggctcttaaaactaaaacctttgcagtcgggcatggtgccacacacctttggtcccagcactggggggtaaggcagaggcaggtggatcactgagtttgaggtcagctagtgctgaagagtaagagcctgtctttaaaacatctcaacagctgggcagtcgtggtccattcctttaatcccagcacttgggaggcagaggcaagtggatttttgagttcgaggccagcctggtctacagagtgagttccaggacagccagggatacacagagaaaccctgtctcaaaaaaccaaaatataaataaataaataaagattgggagagaagtcaaggatctcataggtggtcagggagctacaactgcagtagtaaagaagtaggacttaaaagaacagggccggcactaattttgaggatctagatcgggccctcaattaaggaacttccttttgtgtgaactcagaatttgaaatgaaatgtgcttgtcagaaccattgcatggcttattttttaatgaaaagtctggctagtatctgcttatcttccagcttccagctcaaagttaaggtcatagatcaaaagaactatgtctttatcttagttgtatcttaatttttattagaattgaatggttttcctaatgtttggtaacatcaaaggtgtgtaagtaaaagtgagaaatcaagattaactttctcttggcaaagattgttgacattggtgacatcttggaccaaatgagaattgttttacttttaaatgtcccatcaacagctctcagttaggctgttctatctggtttgtcttgccatgcttgcagagtatagatttgacaatttgaaaattcaaaaagctatataaataggtatgttgctatatgtaagattttaaatgagtcagttaagacttaaagaataactgggtttattttatcttgtcaggttatcactgtgtagaccaggttgaccttgaaaacaaattctctgcttcccaagtgctgggattaaaggcgtgtgtcactagctctgacactggctactttggaactactatggtgttcacaaatgcagagttgagtgttgggattaaatggaaatttcatgtctttttttttaactttcccttctacacagggcttctctgtgtagtcctggctgtccttgtagctctagactaggctgaattcaaactcagatccacccacctaagtgctgagattaaaggcatgtgccaccactgcccagttctgaattgttggggttttttttttgttgttattcttaattttagttcgatgaattaaaatcgaaataacttgtttcttagaaaaataaggtgtaattgggttataaagccaaatttagacattaataccaacagcctgtttaggctcaaaattgttcaatcattttattagtattattattaatcatatcaacttgagacctgtttgggaaagcagaatattttagggatagctatttcagacaagcattaatgtgttagctgtttttttccccctagaatatgattaaaattggctcagggtggggccttctagttctggctctagcgattgggtctgtttctggtgaggtggtagtgataaactgtaacagaagggacaagagattgggcttctgagaacatgtatgatctggtatgtgactttaatcattaaagcatggggattcaaaaatactaatgaataggccttagaactagtcctgagtgttttgtaaaataacagtcttaattctcctagtttctggatttttttctttgtctttggatactaagtttaagcatttatttggacagagtggtgcccactagctgttctattctagtacttgggaggcaagaggcagaacgaacatggccagcttggacaacttaagaaaactgaaaggcttgccatcctagtttttcttttgattaaacacacttatattctaactagttttctctatcctttgggttttgttttgttttttatttgttttgtagctctagctggctggcttcggaattgcctgcctcccaagtgctaggattaaaggcatcacagtcactacccaattgatatcatgattcttaattcaacttctaagaacaaattatcacactctgaatctaacatggaatagcattatccatgttcagatatctttgtctcaaggctcaggtttatcctttgtagctttctttttgctatcccacctctattcagcatccagtcaaggatcactgagttggttatcagtaaattaaacattttaattaatgtctaggagacaggttatggtatagttctcagtgctgggaagttgacatggtaggatctcagttcatggccagccagaactttctggtgagatcctggttcaagcactacagagcttttctagaaaagtaactatatttaggagtaagtttgatataatgacaatcccatcgtaagccttcagtaacctgatgcattggtctctgttttaatatcaggtactcccttgtagcagctgagaatgatggatcaagccagatcagcattctctaacttggtaaggtactttcatctatctgaaaaaatgttcagtaaaaaacaaaacaaaaacatgacttcacccagtgagtacaagatgatacccccaattttttttttttttattatatgtatgtacactgtagctgtcttcagacacaccagaagagggcgtcagatctcatcagatggttgtgagccaccatgtggttgctgggacttgaacactggacctttggaaaagcagttgggtactcttacccactgagctatcacaccagccccaataccccaaattttgaccagccttttttttttttttttttttttttttaaatctgaactaatggtattaaggtgaaacagattgcacaaaagaggtactaggtttttctttgaggcaaggtctttctatattgcctcaggttgaccttaaactccaacttcctttttttccccaagtgctggagattgtagacagatatatacaacaccccaaaacaaatatgtttagtttcgattaagattcattatgtggggctagagggatgacttagaggttaagaacactgcctgctcttccagaggtcctgagttcaattcccagcaaccacgtggtggctcacaaccatctgtaatgggatccgatgccctctttctggtgtgtttgaagacagctacagtgtcctcatacataaaaaaaataaacaaacagatcttttaaaaaaaaaggtacagtgtacttatacattatataaatgaatgattctttaaaaaattaattatgggaaatacttatgaagaatagggtagctttggctgttttggaaacgttatataacaaggtagaactaaaatgtatgccagtaatcccagaggaatcattagccagtcagggctagtctgagcaatgtggcaagataaacccatctctttaaaaaaaaaaaaaagtattataaatagaaatgttataggaaacaggaaatagaaaccctcgaaaggctgaatgaaagagtattagtgggctggaga

SEQ ID NO: 8 - DNA, 2021 bp, Mus musculus
  source 1..2021: mol_type = other DNA
gctggtttgcatttccctaaattagacagtaactttcagaagagatggaacttgttttcttgccagcaaggttgaaaataggtccttagctggaccaatttgtctgtttacaggaataagtaaggccttactggttaaccttggtgttctttatgatgagaccagaagccaaagacctcaagttttctctcctcttggcatctgccctatagtctttagttctttgtttttattttgtttttctttttttcccaacattttctgaaaaagaacaagttttagactcaatttgtcagacttgaaaagaacacactgccaagttttggccaaagtgttagtcttcaggaaagctttctatcattttggcactgagatatttattgtttatttatcagtgacagagttcactataaatagtgttgttttttttttatatatagaagataattatcggaagcagtgccttccataattatgacagttatactgtcgttttcttttaataaaagcagcatctgctaatgagacccacagatactggaagttttgcacttatggtcagcacttgcaactttaggaaggaagaatgccacaatccaaataatatgagctagaagaggattgggttaaataagagattcctaattgagttggggaaaaaaatgatagttttcctaagtgcagtgagttgtggccaagttaaatgtcatttaaaggctatggtagtacttacatctacaaaattttacagctcaatttattcaagatgtaactcaaaatcaattttgcaaaatttccagtacctttgtcacaaacttaactcacattatcgggagcagtgtcttccataatgcataaagaacaaggtagtttttgcctaccacagtgtctatatcggagacagtgatctccatatgttacactaagggtgtacgtaattatcgggaacagtgtttcccataattttcttcatgcaatgacatcttcagagcttgaagatcgttagtatctaacatgaattcctaacttcctgtagctccttagttttagttgcaaaaaacatttgtggtcattgagcatttggtgggtaaaatcaactgctgtaaaatcattacttccaagatccttttttaaaatgtgggtattttgttatctctggtttatggaataaaagcatacgtttataatgtttgctggtgacaaaggaatttaactatctccctttatttgcatgttacatgttatttcctaaatgtagttcttagaagtttgaattcatttttggtttttgggagggtagaaagtagagaataatgggtgttgggaagacaaagcttgcaaaaggatctttatgtacctgctcatcactatggtggctatgaattatgtaggtaatgagcaagatcacattaacaaagactagttaaccatcattacgcatctaatcagttttgccatggggtcagttcaaagctgccacctgagaatgtcactaggctctcagggtcttggcaccactcgcccaagttatatccaccagattattttgagcctcactgagttgttttgctactcagtcccttcagtcttcacagtgtttctcttgtaccagtatgagtatcaccgtgcataatgggattccaagtcttgaatgaaggcatgcattttacatagacgtctctgtgaaataacctttgttgtaagtggtgtttccagtcaaagcagtaagttggtaaggtttagtcttgggtgaaatgtggatctgagtaataagtagcttctgctacttatcaggttattgttggttaaaatgtagattttgaagataaaaatagatcttttgtcatgactcactgctagaatttgcatgacctttactgtgttagcttttgaatgccttttggtttggacttgccaacctatactgtctttgctgaaatgcccttataaatgtatacagctggtatgtaacaatgtgaagattccttacctgacttaataaaatacttcgataatactga

SEQ ID NO: 9 - DNA, 38799 bp, synthetic construct
  source 1..38799: mol_type = other DNA
cagagggccggccggggggtggggccgagctataagctttgggtgggaggcagcgctgccttcagaaggcgtgcggagcgcgggctgctgcattgcggactgtagaggcgcttcctagtgagtgactcccttgtcagcggcacggcccatcgtggtcctcgcgtggcgggcggaccagagcgagacgccagggcctgggtggtgcgggcggggaggcggaggggtgtcgcggagtccggggctgaggagcgcgggttgcaggtgcagcgcggtgggtgtggggagccgctgtaccctgcgcccctcgggtcctccgggccttcgcaggccagtgctaggccgcgggttcgagagtcaccacgctgaggcgcaggcttgttccgccgggagcacgtggtggcggctggaggaagtcgccccagggaacggctgtcggggtacgtgggtgaccttggggcccctcgcaggagggcgtcacagctgaaaaggacaaagctgttttctattcggttactagtgtcacggacatttagaggggcggggggagcttccaataactgcacgttggaacttcggcaccacctggtcggtttttttgccagtctctccctcttggcccagcgtgtggaatctcatttttctagggcagaataggtctgaacgctgcaggtaatactaagaacgtctctagcatctcctaagatgggagaacgtagaaatacgacctctttgtacgagctcttttagaactagctgtagagaaccagcgtgcaccctggtgttggacagctctctaaaatggtgtttgagggtaagaaaactgcatttgcaaatttttcagttagcacactttgtcccgagcgtaaaatgaaatgatctccttatacttaggcagaagactggactggattgctgttcagtttctgtgctatttttttaaaataggatttaagtgggaaatagttgtgttacagaaattctcggctattctgtgctatttttttttaaataggatttaagtgggaaatagttgtgttacagaaattctcggctacctgatacttttattctaagattagatgagttggctctgagctgtgaaatatgacctctttgacaaagacatttaagctgattcaggatgttatctacaaagaaaacgggatttagcttgtgtgggtccacttgcatttattttctgttaaggattataataaactgctttatacaggaatccaatatcagctgttttttatatagagagcataatactttttactttgagagaggatgttgtcaaggaatggtggctgtgaacctggcctgtgttgactggttagatctgtctgcctaaccccaccctaaggctaagtagttatatgcttgtggcaatgtgcttatttataatagggcaagattatgggctaaatttgggttagacaacaatgaaagttaattaaacgaccctcaggccttgggtctactatgtgtaagtgatttccttctctcccagatgagtgctatacaaaataaacttcagtgacctcaagtggttttgaccttttggttgctattcagaaaactatggaaatgaaaacctgctacctatttcctattgccttttcaatttcccaaagaaggtctcccctatgaatccatgggtagccttgaactcagatccacctgcttctgtttggagagtgataagattaaaggcaagtgccaccacacccagcaaagtaggctcttaaaactaaaacctttgcagtcgggcatggtgccacacacctttggtcccagcactggggggtaaggcagaggcaggtggatcactgagtttgaggtcagctagtgctgaagagtaagagcctgtctttaaaacatctcaacagctgggcagtcgtggtccattcctttaatcccagcacttgggaggcagaggcaagtggatttttgagttcgaggccagcctggtctacagagtgagttccaggacagccagggatacacagagaaaccctgtctcaaaaaaccaaaatataaataaataaataaagattgggagagaagtcaaggatctcataggtggtcagggagctacaactgcagtagtaaagaagtaggacttaaaagaacagggccggcactaattttgaggatctagatcgggccctcaattaaggaacttccttttgtgtgaactcagaatttgaaatgaaatgtgcttgtcagaaccattgcatggcttattttttaatgaaaagtctggctagtatctgcttatcttccagcttccagctcaaagttaaggtcatagatcaaaagaactatgtctttatcttagttgtatcttaatttttattagaattgaatggttttcctaatgtttggtaacatcaaaggtgtgtaagtaaaagtgagaaatcaagattaactttctcttggcaaagattgttgacattggtgacatcttggaccaaatgagaattgttttacttttaaatgtcccatcaacagctctcagttaggctgttctatctggtttgtcttgccatgcttgcagagtatagatttgacaatttgaaaattcaaaaagctatataaataggtatgttgctatatgtaagattttaaatgagtcagttaagacttaaagaataactgggtttattttatcttgtcaggttatcactgtgtagaccaggttgaccttgaaaacaaattctctgcttcccaagtgctgggattaaaggcgtgtgtcactagctctgacactggctactttggaactactatggtgttcacaaatgcagagttgagtgttgggattaaatggaaatttcatgtctttttttttaactttcccttctacacagggcttctctgtgtagtcctggctgtccttgtagctctagactaggctgaattcaaactcagatccacccacctaagtgctgagattaaaggcatgtgccaccactgcccagttctgaattgttggggttttttttttgttgttattcttaattttagttcgatgaattaaaatcgaaataacttgtttcttagaaaaataaggtgtaattgggttataaagccaaatttagacattaataccaacagcctgtttaggctcaaaattgttcaatcattttattagtattattattaatcatatcaacttgagacctgtttgggaaagcagaatattttagggatagctatttcagacaagcattaatgtgttagctgtttttttccccctagaatatgattaaaattggctcagggtggggccttctagttctggctctagcgattgggtctgtttctggtgaggtggtagtgataaactgtaacagaagggacaagagattgggcttctgagaacatgtatgatctggtatgtgactttaatcattaaagcatggggattcaaaaatactaatgaataggccttagaactagtcctgagtgttttgtaaaataacagtcttaattctcctagtttctggatttttttctttgtctttggatactaagtttaagcatttatttggacagagtggtgcccactagctgttctattctagtacttgggaggcaagaggcagaacgaacatggccagcttggacaacttaagaaaactgaaaggcttgccatcctagtttttcttttgattaaacacacttatattctaactagttttctctatcctttgggttttgttttgttttttatttgttttgtagctctagctggctggcttcggaattgcctgcctcccaagtgctaggattaaaggcatcacagtcactacccaattgatatcatgattcttaattcaacttctaagaacaaattatcacactctgaatctaacatggaatagcattatccatgttcagatatctttgtctcaaggctcaggtttatcctttgtagctttctttttgctatcccacctctattcagcatccagtcaaggatcactgagttggttatcagtaaattaaacattttaattaatgtctaggagacaggttatggtatagttctcagtgctgggaagttgacatggtaggatctcagttcatggccagccagaactttctggtgagatcctggttcaagcactacagagcttttctagaaaagtaactatatttaggagtaagtttgatataatgacaatcccatcgtaagccttcagtaacctgatgcattggtctctgttttaatatcaggtactcccttgtagcagctgagaatgatggatcaagccagatcagcattctctaacttggtaaggtactttcatctatctgaaaaaatgttcagtaaaaaacaaaacaaaaacatgacttcacccagtgagtacaagatgatacccccaattttttttttttttattatatgtatgtacactgtagctgtcttcagacacaccagaagagggcgtcagatctcatcagatggttgtgagccaccatgtggttgctgggacttgaacactggacctttggaaaagcagttgggtactcttacccactgagctatcacaccagccccaataccccaaattttgaccagccttttttttttttttttttttttttttaaatctgaactaatggtattaaggtgaaacagattgcacaaaagaggtactaggtttttctttgaggcaaggtctttctatattgcctcaggttgaccttaaactccaacttcctttttttccccaagtgctggagattgtagacagatatatacaacaccccaaaacaaatatgtttagtttcgattaagattcattatgtggggctagagggatgacttagaggttaagaacactgcctgctcttccagaggtcctgagttcaattcccagcaaccacgtggtggctcacaaccatctgtaatgggatccgatgccctctttctggtgtgtttgaagacagctacagtgtcctcatacataaaaaaaataaacaaacagatcttttaaaaaaaaaggtacagtgtacttatacattatataaatgaatgattctttaaaaaattaattatgggaaatacttatgaagaatagggtagctttggctgttttggaaacgttatataacaaggtagaactaaaatgtatgccagtaatcccagaggaatcattagccagtcagggctagtctgagcaatgtggcaagataaacccatctctttaaaaaaaaaaaaaagtattataaatagaaatgttataggaaacaggaaatagaaaccctcgaaaggctgaatgaaagagtattagtgggctggagaggagagactgactgagaagaccagtgtgaactagcccttgactgggcccagggaagaatgttggtttgaaaggagaacaccctaaggaatatatagcaagttatagtaatggcccaataacaagcatatacctaaacgagcttttcctaaacattcccgtgcctgttcttcatcatgcctttttccgcaacacagtttggtggagaaccattgtcatatacccggttcagcctggctcggcaagtagatggcgataacagtcatgtggagatgaaacttgctgtagatgaagaagaaaatgctgacaataacacaaaggccaatgtcacaaaaccaaaaaggtgtagtggaagtatctgctatgggactattgctgtgatcgtctttttcttgattggtgagaatgaccattccaaacttcaatgttttctataaccaactctgggaagtctgttatgtcagctcagcatatagttattttgtaccttattttttttttttttttttttggagacagaggcttactctcttgcctaggctagagtgcagtggcaccatgttggctcactgcaacctcagcctcccaggtttaagcgattctctgccttagcctcctgagtagctgggactataggcgcccacgaccacacctggctaatttttttttttttttttttgagttggagtctcactctgtggctcaggctggagtgcagtggcgtgatttcggctcactgcaatctccgcctcccaggttcaagcgattcttctgcctcagcctcctgaggagctgggactacaggcgtgcaccaccacacctggctaatttttgtatttttagtagagacggggtttcaccatgttggtcaggctggtctctaactcctgacctcgtgatctgcccgcctcagcctcccagagtgctgggattacaggcatgagccactttcccgtccactttatgcctttttaagtgccactaggaagacaatcatgagtagattctatacctgtcattgaatttaatctggttggaaagagagacccatgaattatttcaatacgacgtgcaagtgttttctttttatttatgtatttttttgtttgctcccactccaccaggaagcaaatgctacgctagaaaatatccgcaggacactggcggtgcagcagctcagtctcatgtttcaagggggtcttttggaaaaaactacctatgttttgaataggtagctatttgaaataaagctagtatataatttattaagtggtttaaagtatacaaaactgagatgcattatatactttaaaactacttagaatttgatgaaaaccagtattttcaaggcttacctgaaaatattaacagattaacagggatagattacaaatgaaattctgagttcagctctggaactttgtctctttaggatttatgattggctacttgggctattgtaaaggggtagaaccaaaaactgagtgtgagagactggcaggaaccgagtctccagtgagggaggagccaggagaggacttccctgcagcacgtcgcttatattgggatgacctgaagagaaagttgtcggagaaactggacagcacagacttcaccggcaccatcaagtgagtgccagctgctgtgcaagtatctagacaagtaattcaagaattatgataggccacggggaaacaataatggtgacactgtggggaatggcttgttagagaagacaagacttgtcatgtttagctaaagaggggaaggggcctgtaaaaaactgaaactatactggtaggggaaaaggtagaacttctctggggagtctcctttttcttaaaaaattaataggcccttgatgttttccccatctttgtagcttattgcttgctgtcattctctggctctgatttttcttcttaacctcttgtaacgttgtactaagctctgctttgctgacaaggacatcagtagcttttctctgttttaggaagcaacacatgccagaagtagcctttttcatctccttgatcatgggtaactgcctgccttaagtctctagtgcttcttaatttctgtgatactcacccattaacccctaggccagtcagtatccagagacaatgtctccctctggtggaggctggaatacggtggcgtgctctcatctcaccgcagcttcatccccctaccatagcctcctgagtagctgggactataagcacgcagcaccacacccagctaatttttttttttttttttttaagagatggagtctcaccatgtctcccaggccggtctgttcttagatttttatttatttacttatttattttgagatggagtcttacgtgttgcccaggctggagtgcagtggcacgatttcagctcagtgcaacctctgtctcccaggtttaagcaattctcctacctcagtctcctgagtagctgagatcacaggcttgtaccaccatgctctgctaatttttgtgggttttttttttttttttttttttttgcagaaacgggatttcaccatgttggccaggctggtgtctaactcctgacctcagatgatcctcctgtcttggctcccaaagtgctggaattacaggatgaggcactgcacctggccaaatttctattttatatttatttatttattttatttgtaatgggctttctggcaaaaactagaaagcatgctagacaaattctaaaagagctgtaacacttgtccatagagttttactatctttgcacatacatgcctctgtgcaaagaataaatttatcttttcctttttgcaagcttcctacacttacagtggtactataattctttagtcttaagttatggcattttctacttcatctagttgatgcttttgtgtttatttactgtacaccagaccttatggttaaagtgagagtatagtccaccccacaaaaatgtaagaactaatatgctgagtaaatagaaagaagtatgggagatcggaccaaggagcagttctgcttcagggtattcagatgcttcacaagtaggtgagaattgaaactatgcactctgttcagcacagtattactatcttagattttatagtagatctggaagctactcatttcccctaaaactatcctaagcagatacattgtgttatgatgaggctcttgaaggagtgctttgtagtgggtaaagaagctttgaagttacaagatttggggcaaaattttttttcttttgtgatattctggtcagtctagaagttttgggtcacagttcatttatctgaactatgagggataatattttgctttcaaaacaatttgtttaatccttgacttaaatgtttattttaaagtaaacttaagttcatttaaaaggcattttctcctccaggctgctgaatgaaaattcatatgtccctcgtgaggctggatctcaaaaagatgaaaatcttgcgttgtatgttgaaaatcaatttcgtgaatttaaactcagcaaagtctggcgtgatcaacattttgttaagattcaggtcaaagacaggtatgttgaaagatggtaaacttatttttatacaagtagctattttcaggtgtgctaaatatagcaaagactttttgttagtgttgttggcttttttttgaaatggagtctcgctctgttgcccaggctggagtgcagtggcacaatctctgctcactgcagcctttgccttctgggttcaagtgattctcctgccttagcctccagagcagctgggattacaggcgcacgccaccacgcccagctaatttttgtatttttagtagaggtgggatggtctcgaacttttgacctcaggtgatcagcccgcctgggcctcccaaagtgctgggattacaggcgtgagccaccgcatgcggcccaaaggcttttaaaataaaaatcaagcttaagatttagaggtaaatttcctcaagccaaataatgcaagacatactaaatgtaagctattgtgttttttggaaggatgaccttaggcttattttaacttaatctttttaacagcgctcaaaactcggtgatcatagttgataagaacggtagacttgtttacctggtggagaatcctgggggttatgtggcgtatagtaaggctgcaacagttactgtaagtaaagcaaaacagtgcatgagactcttccctattgaatcattcaaaactcatcttttctgttcttaggagttataaatttacctgtaaaatgtaaatgatcatgagatattttggttttcaaccctctaatgacacagtcaacatgcattgtcttctctctctaatcactttccccatgtcctgttttattttttctttatagtactgctagcagctgacattatctatgcctttgtttccattagaatgtgagccagatgaagaatcatgggttagttctatttactgccatgttgcagagctttggagaatgcctgacatacatagtagtatttgctaaacaaatgcatatcctccctgtggggacagatgtaaatatccatcttggctggggcctagtcttagcttgattcgttaatgcttgatctttttctatttttgttttgagacagggtgtcactctgttgccctagctggagtgcagtggcgtgagcttggctcactgcattctccaccaccacccaggctcaagtgagcctcttgagtagctgagatgtccagttaatttttattttttattttattattattattattattattattattattatttggtagagacagggttttgccatgttggccaggctggtcttcaattgaaactcctgggctcaggtgattcaccaccctcagcctcccaaagtgctgggattacaggaggaagctgcttcgcccagcccttgatctttttaaatttaggcaaatatagtcaaattaacacagaaatagagaacagatagatagaataattttcagcttaaaaatcacatttgtggctgggcgtggtggctcacgcctgtaatcccagcactttgggagtccaaggtgggtggatcacctaaggtcagagctaccagcctggccaacatggtgaactctattaaaaatacaaaaattagccaggcgtggtggtgggtgcctgtaatcccagctacttgggaggctggggcaggagaatcgcttgaagccaggaggtggaggttgcagtgagcggatattgcgccattgcactccagcctgagcaacaaaagcgaaattccatctcaaaaaaaaaaaaaaaatcacatttgtaagtcaggcgtatctctagtggatacctttcggggctggtgcagttctgtgttgattccttgcctttctggatacttgtgctttatccctttgcctggctgcctataagccaggagtttagagatgggtaggttgttctgttaaagaacattgaagatttttgttcaacttcaagctttctgtattgagggcttggttgttagtgtcagtttatggctgttcatctggactcttatgagcttaggaacatcatgacacatctaaagttggtgtatgcaagtcactttgttgtaatagatggtgttttaattttgggacagatttattatcagacctttcagttaaaacctatttctatgatgtcctcaaggctaagattgttccaggcctccagtcccatgaccagcctacataggcctctttaatctaaatgcccttcagatgaaaggcagcatgggataaaagcatgaaagacataggaggccaaaaatcttgattttattccaatagctctgacctaaaagttgtaaagttttttagaactcatgtcttttttaggccttggcctccttttatctcttacttgggttgcctgtcttaggtttgatctataacactcttctgtctcaagattttagagctgttccgataccataatgctcattaaatctaatgtctttgttctagggtaaactggtccatgctaattttggtactaaaaaagattttgaggatttatacactcctgtgaatggatctatagtgattgtcagagcagggaaaatcacctttgcagaaaaggtgagtatgagttattataatattaaatacagtacttttggtatcttactcttcaggtaagtgatatttcttgttaatattcttacattctgaaaatcagcaatcttaaactctcataccagataatataatttattaaaatgctcaacaaatgaagtaagttccacttacctaatcagaagtgcttaagtgaaagctatttctctgtataactcagttttacgcatatacagaattcagctaaaagcatgttttataaaaaggaaaattaatagccaaacatacacaaggaactagagtggaacatatcaaaagacagtggtgatttctgcatttttaaaaaaagtggcagggctcacacctgtaatcccaacactttaggaggctgaggtgggaggactgcttgagccctggaggttgtgagatgtgactcgtgccactccagcctgagtgacagtgagactcaaaaaagtggttgtcttagatgggtttttgttatttttcttttcttttttttttttttttttgagacggagtctcttgtcaccctggctggagtgcagtggcgtgatctcagctcactgcaacctccgcctcccaggttcaagtgatgctcctgcctcagccttctgagtagctgggattacaggtgcccaccaccaccatgcctggctaatttttgcatttttagtagagacagggtttcacgacgttggcctggctggtcttgaactcctgacgtcaggtgatccacctgccttggcctcccagattgctgggattacaggtgtgagccaccactcctggcctttgttattctttttttttttttttttttttttttgagagggagttttgctcttttgtccaggctggagtgcagtggcgcaattttggctcactgcaacctctgccttatggtttcaagcgattctcctgcctcagcttcccaaatagctgggaatacaggcgccagccactgcgcccagttaatttttgtatttttagtagagacagggtttcaccatgttggccaggctggtctcgaactcctgagcttgtgatccccctgcctcagccttgcaaggtgttgggattacaggcgtgagccaccactcctggcccgttactcttattttgcaaacaagtgagttaatttttcaggaagcaattaataaattaccagataaacaatttgaaatatttggaagtattataagtttagtcctctgtaaccatttggcctttagcataatgtccaggatgtattctcatttcgtatgatcagatcttcattttctgagctttatatgttttcttttaggttgcaaatgctgaaagcttaaatgcaattggtgtgttgatatacatggaccagactaaatttcccattgttaacgcagaactttcattctttggacatgtgagttatttcttgagtaaatcaccgttttgagttccttgagttgttcttggattcctgtattagcagaaatagcactgtgtcttccttaactgctcttttttctgggagggcgttagcagtaagcaagaagatagattacattgactttgaggctttattatttgttgctaaaagtactttgtcaatagtgccttgaatttgagaatttccatatgccatgaaaacaggagtcacattgtagagccactcagatttttgaggcttcaagttggtaaactaaggttgggctgcagccttataccaaacctgaatcttacagagaagttttgaggaagtatgtgatggagtgaattgctcttttgttttcctaggctcatctggggacaggtgacccttacacacctggattcccttccttcaatcacactcagtttccaccatctcggtcatcaggattgcctaatatacctgtccagacaatctccagagctgctgcagaaaagctgtttgggtaagtttttatttgaaagcggtcttgcatagtgaggttttatgattagggaagaacggtaatatgttgattaaaatgttaaacatgatgataattttccacatttgggaatttggaggatggttagctgttaccttggtacagatataaatgaatttttctctataaagagatcttaataatggactttggacttataaacacaggagaataaggagacttataaacccagtgaattccttgtgcaagattcctggcatagcttctgctcaagtacttctgtgctgaagcacgatgcctatagcagcccattcagttagtagagagaatctgttagaattcatattgggataaaatcctattccccgaaacatccatttgtctggatttttcttctaagcaatacggtatgattccagttaccttgtctatagattgagaagccttgcttcttatgatggtttccagagaaaccatttatggttgtggctcgctcctctggatatcctccctgtaacgttagtgtctctctttaaaaacagagcccagttccattagagtacgtagcattaattttgatttggatgtggacatatttatcatttcctgttattacaggatggagatctagtttcaggcctttatgaaagcatttatctcctgtatgacataggttctctgatcctgtttctttaacataattggatagtgaaaatatattctacttgatagtctcaaatgaagaacatctgtatataaggagtatatgaaataccatgactgttgatcatgctgagaggcctttagcataggggagtgtgtaacataccatgactattgataacgctaagaggcatttagcataggtgatttttttgactcattctacctaccacctacaaatcgtaaccaacctgtgaagccagcgtcccatcttatccttatgtagtaggggagtaataatgtttcaacctatatttatgtaagtaagcccccttaggagcagctattatttgggtaacacagaggaattagataggggaagcacaagattttttttttattttgacagtctcactctgttgcccaggcaggagtgcagtggcatgatcctggctcactgcaacctccacctcctgggttcaaggaattctgcctcggccttccaaagtgctgggattacaggcgtgagccaccgcacccagctgaggaatattttttataactgagctaagaatgtgtactatccttgttagtggtgacagttgggaaacataaaagtgtattaatattcttttatatattagaagaacttcattttgagtccatcttggtatgtattccaaatataaactacgtaagtatgtctggcagaaaggcgcatagttagagaagtgttttaaaatattgcttaattaatggtttccaattggctgcctgcagatcaaagtaagacgcaaatggttcaccactagagagtaagtttttttttgtttttattttgttttgttttgttttgtttttttttgagacaggacttgtgctctgttgcccaggctggagtgcagtggcgtgatctcggctcactgcagcctccgcctcccaggttcaagcgattctcctcctcatcctccttagtagctgggattacaggcgcatgccaccatgcccagttaatttttgtatttttagtagagtcggggtttcaccatgtcggtcaggctggtcttgaactcctgaccttgtgatccacctgcctcagcctcccaaagtgctgggattacaggggtgagccacttcgccgggctgagagtaagttttgtttatatgtcctcttaatctgtaacttcactggacaggagtaaaccctgggcaaggaacaataactcagaacttacgcctgctttctgattctaggaatatggaaggagactgtccctctgactggaaaacagactctacatgtaggatggtaacctcagaaagcaagaatgtgaagctcactgtgagcaatgtgctgaaagagataaaaattcttaacatctttggagttattaaaggctttgtagaaccaggtaaagaccgcccccccccccccccgccccgctttttttttgttttcttttctgttcctaaggatgtggctagagaaggagcgagtgtaggaatgctggcttggcttggttttatgaagtgctcaatcttgtctgtcctaaagttaattgtttatgtgttagtttcttttttttttttttgagaaagagtttcactcttgttgcccaggctggagtgcagtggtgtgatctcggctcactgcaatttccgcctcccaggttcaagccattctcctgcctcagcctccccagtagctgggattacaggcatgcgccaccacgcctggctaattttgtatttttagtagagacagggtttctccatgttggtcaggctccatgaggtcagagctcctgacctcaggtgatctgcctccctcagcctcccaaagtgctgggattacaggcatgagccaccgcgcccagcctatgtgtcagttttattgggaggtaataaggccctaggaaattcttgttaaaaattgaccattatgactagaaaatctattctaagttattgactagaccagtgataagcaaatttcttaagggggcagataataaaccttttgccaggctatacagtctctcttttagctgttcaactctgtgggagcaagaaaacaggcacagactgtgcgtagtgaatgggcatgcgggctgtagtttgccagtccctggagttaactttaaatgcaatcaaacagccaacacttactaagaaataagtccttggacttaagtagtcagtaaatttaattagtagtgtaggaaaaaagtagctctactagtaaggtaaaattaatattctcatgtgaatttttaattcccagagtttttagtcagatttgaagtaaggctcttttatccttaatacatatactgtgcttttacctttctactgatgtgctaattctagaaaagtttagaagctgattatataagttctttcttcctcttttttcttttttaaagatcactatgttgtagttggggcccagagagatgcatggggccctggagctgcaaaatccggtgtaggcacagctctcctattgaaacttgcccagatgttctcagatatggtcttaaaaggtagagtacaaattttgattcttttgaatattggtgcactgcatacagttctagatgttatactgtgctttgctcactttgcctgcattcctgtggttctcatgctagactctagttcttaaatacaaggcagattgtcttttgttgtagctgccttttcctttgaaacagctttccataaagtgtcttaactgtgctagaaatcaccagtttctttgagacagtggagttactgagccctagtgcttagtgtggtggatccagagtgataaggtggatcatagtccctaagcaatctatttgaaagcagtagcatacctctctactcatgtactgaggcctaacagctattgaaattttgttctgtttattcatttattcattttttgagacagagtttcactctgttgcccaggctggagtgcagtggtgtgatctcacttagctcactgcaacctctgcctcccgacttcaagcaattctcctgcctcagcctcctgagtagctgggattacaggcacccacaaccacacccggccaatttttgtatttttactagagacagggtttcgccatgttggccaggctagtctcctgacctcaagtgattcacccgcctcagcctcccaaaatgctgctagtgagccaccgtgccctgcctcttttgtttatttttgagcctttctgtcctactttccactctttcacactcctacccacccacacattcaaaatcacgtcacattcttgaatctttgggtcttagggcaaactgcaatgcttaactttaatacagagtacattaatggagaaaaagatacagtgagggaagtgggagggatggagggcagtgtgatacatgtaacctcaaaaggtgctgtcacaaatatagtttctctgcaaactcttcttgtaacttaaatttgtgaatttttattttattttagaattatttttttaaaaatccaatgtttggatttacctctgaagatttttctcaagatgtgcatcatcatgcttacactgttttcaatgatctctgggtccagaagttaaggaactccaagaatgaaattgtgaaagggtgactactggcccctgctatgtactcaaaatcttatttgcagatctcctatcttgctctttgtaggttactttaaaaaaaattattcctattcttttattcttttacctggggtctcaaaaaaagattttgttagttttttttttcttttttggctcagaaaaaaaagattttggaatctgctatcttggaattagaccttctgattcatcttaagtgggataccttccatccttgtctgtgtataacctttcctcaggtaaagctaacttttttctctttcagatgggtttcagcccagcagaagcattatctttgccagttggagtgctggagactttggatcggttggtgccactgaatggctagaggtattctttatcatcccttcccatattggacacgagcttgtgggcttaggctgttgtccagaagtgatgtttttataggtttgattttaccacttttgcctttgcgtttagtctcagtagagtccagaattgaaaatgaatccctaatgctactgtatgtgataaataaacagatttatacttattagtgttttcccttctcttctagggatacctttcgtccctgcatttaaaggctttcacttatattaatctggataaagcggttcttggtaagtatccctttcattagctgtgtatgaattcaggtaaacttttttgagatggagttttgctcttgtcgcccaggctggagtgcaatggcacgatctcggctcactgcaacctctgtctcccagattcaagcgattctcctgccttagcctcctgagtagctgggattacaggtgctcactaccacacccagctaattttttgtatttttagtagagacagggcttcactatgttggccaggatggtctcgaactcctgacctcaggcgatctgcctgcctcggcctgggattacagatgtgggccactgtgcctggccagataaactactttgaagtggaagaaagcttttttttttttttgagacagggtctcactgtattgcccaggctagagtgcagtggcacaatcttggctcactgcagccttgacctcctgggctcaggtgatcctcccacttcagccacctggctaattttttttgtagagatggggttttgccatgttgcccaggctggtgtcaaactcctgagttcaagcattctatgtcagctgcccaaaatgctgcggttacaggcatgagccattgccctcagcctgtatcttaaccttcctttaaatagtctgtcaagttacacagtgagcacaattgcttgtctagaacagtgggtagttctcagtgtggcccccagatgagtagcattaggaactgttacgaatgcaaactgtcatgtctaccccagacctttgagtcagaaatgggagtgttggtctaaaaactgggctttttgggctgggcgcagtggtgcacgcctgtaatcccagcacttgggaggccaaggcaggcggatcacctgaggtcaggagttcgagaccagcctggccaacatggtgaaaccccgtctctactaaaaataccaaaattagccgggtgtggtggcgagtacctgtaatcccagctactcaggaggctgaggtaggagaatcacttgaacccgggaggcagaggttgcagtgagccaagatggcgccattgccctccagcctgggcgacagagcgagactccatctcaaaaaaacaaacaaaaaaactgggctttttttttggcaacccttggggaataaaaacctattattttcttaaggacaaagtatcctgaagcaaaaaaacccaaacaaagaaacaaaaaactttaacaagcactacaggtaattctgatggactaaagttttaggaccataagtctggactatattgaggtgagaagaaactaaactatgccatatagaatggtacttagagagtaattcacatcctgttacgttgtggcatcactgatagaaatattggataatgaaacttctagaagagtttgaatgttcactggcagcagaaaattgacaaaagggtttgaatgttatttaaagtgcagctgtacattcaacaggaatggggtaaaaaagaaagtgcagatgtagctgatgagctgagaatagtgaaatgtctacatgggggaaaaaaggaaagagttacaattaaacctctctaggttagttatttccctgttgtatgtttgccgcagaatgtgctgagtatagcaagcatactatgtatagctctaacctgggtgaaccagaaagttaaacatgaaattgctaatgggggaggctgcacatgtgtgggggcagtggatttatgggacctctaggtacctttctcttaattttgctgctgaacctaaaactggtctgatttttttttttttttttttttttttttttgagatggagtctcgctttgtcacccaggctggagtgcagtggcgccatcccagctcactgcaagctccgcctcccgggttcatgccattctcctgcctcagcctcccgagtagctgggactacaggtgcccgccaccacacccggctaattttttgtatttttagtagagacggggtttcactgtgccagccaagatggtctcgatctcctgaccttgtgatccacccgtctgggcctcccaaagtcctgggattacaggcgtgagccaccgtgcccggcctaactgctctgatatttttaaaaaaggtgacttggattaaagtatgcaaactcaaggagtagtacgagcccacttgagtgaagttagcttagttgctaaagagcttgataccaaaattacttttgtttacttgatgaatgggatagttgttgcacagggccactgatctagattactgtcttatttgtcaagtacttatagttgtagaagtagcagtgtgaaaattatcggaatggagaaggggcaatgataaaacaatttattttcaggtaccagcaacttcaaggtttctgccagcccactgttgtatacgcttattgagaaaacaatgcaaaatgtgagtatatacctcattacaaaaatgtatgacttaatttttgttgaatcaacctgagataaaaaacactgatatgtaaaccgtagtcagtaacaaaaaataggaattgagaataaattttatagcagcgttatttaagggatacttgcctattgaaccatatgagatgagggctccaatcttaaagaatatgttgtttatttaggaatatataacaaaatgccatgaggcctaagctgtagtgcaggggcccacaggggattgtctatagagtcacaatgctaaggaaggcttaaatcaacttgaactatactttgagaaggccggaggaaatattcattgaataaacaattaccatagttttgaatgagggagagcatgtgaaagaaaatatttgcctatgtggggtggggggaggggagagggatagcattgggagatatacctaacgctagatgacgagttagtgggtgcagtgcaccagcatggcacatgtatacatatgtaactaacctgcacaatgtgcacatgtaccctaaaatttaaagtataattaaaaaaaaaataaataaataaataaaaaagaaaatatttgcctaatgggattagaggctgtatattggagtgagtaaagttagaaacaggtcagatagcagacattggccttgatccaagagatactagggattataggatctcaagcaaggtgatcatgaatttgccagttaggttggtggcataagatgcactagaaggaagaaaccagaaacaagataggcatagtgtgaagcccagagagggcttttacaattgtgggtaagccaccatggttttgtttgtttgtttgtttgtttttgagatggagtcttcctctgtcttcctccagcactcaggctggagtgcaatggtgcgatcttggctcactgcagcctccacctcctgggttcaagcgattctcctgcctcagcctcctgaggagctgggactacaggtgcacaccaccacacctggctaatttttgtatttttagtagagacggggtttcaccatgttggccaggatggtctcaatcccttgacctcatgatctcttgaccttggcctcccaaaatgctgggattacaggtgtgagccaccgcgcccagcttttattcatttttttttccttttttttttttaagagataaagtcttgctgtgttgcccaggctagtctcaaacttctgggctcaagtgatcctcccagctcggcctcccaaagtgttgggattgcagttgtgaactaccccacccggcctaggcccacttttaaaatgttaattaagaagaaaacattttttcctcaccataactaaaggaactgacaacatgagcctagttagtaataacttaaagttgagattgttttaactatcataaataatttcttttcaagaagatataatacaagttttttatttaaaaattgttatagagtaacatgttagtgactagcctgaataaataagttccaaaaggttagttttaatgataatttcttatcttacaggtgaagcatccggttactgggcaatttctatatcaggacagcaactgggccagcaaagtgtaagttgagaaaagtgaatgaacaaactaatagaaaagcagagattctacctactacattaggtagaatctaaatctgtccttgcattgaacttacttacacctaaagatattcagctaaagaatttattttggatggggggactagcacgatagaacagtctattctttaaaacactttctaaagacacattctttgtctttgcagtgagaaactcactttagacaatgctgctttccctttccttgcatattctggaatcccagcagtttctttctgtttttgcgaggtaagtctgttcatttaaatgacaaatggagagaggctctctaaaaggaagtgatctgtatttgggaatagggcattgcaatgggaatgcctgtgctatagtcaactatgtacatattcagaggagtaaaggaaaacaagtttttaaggacaaatgacgaggattacaaaattactttgaggtaattattcttggctaccaagatcaataacaaggatgacaccagtccaaggttgacaggcagttgctaggcagattttctcacgagagaagttttttggtgcaaggttgcagtggcctttgtgtaaggttgtagtttttgtaaaaaggaaaaaaaaaaacccttaatccttgttatcagacatacaagggtgagacccttctcttcagggttgcatttttgttaacactagtgactccattttgattttgacaacttgcacatatcttagttgttgggttttttttgtttgttttttgttttgtttttttttttttttttgacaatggtgacttgctctgtcacccaggctggggtgcagtggcccgatcatggctcactgcagcctcaagcagtcctcccccttcagcctccaaactgtttggattataggcaagagctacttactacacctggccatggatcttcttgtgtcaactttgagaccaaatttactgcagtgcttaaaatgttttaagaagttatacacatgtgctggatgtggtagctcacgcctgtaatcctgtagcaggacaagccgcagacaaatcccctcagacaccgagttaaagaaggaagggctttatttggctgggagctttggcaagactcacgtctccaaaaactgagctccccgagtgagcaattcctgacctttttaagggcttacaactaagggagtctgcgtgagagggtcgtgatcaattgggcaagcagggggtacgtgactgggggttgcatgcaccggtaattagaacagaacagaacaggacgggattttcacagtgcttttctatacaatgtctggaatctatagataacataactggttaggtcagggctcgatctttaaccaggaccagggtgcggcagcgctgggctgtccacctctgccttttagtttttacttcttctttctttggaggcagaaattgggcataagacaatatgaggggtggtctcctcccttaatcccagcactttgggaggccaaggaaggcggattacgaagtcaggagtttgagactagcctgaccaaaatggtgaaaccccgtctctgctaaaaatacaaaaattagtcggacatagtggcgtgtgcctgtaaccccagctactcaggaggctgaggcaggagaatcgcttgaacctgggaggcagaggttgcagtgagctgagatggagccactgcactgcagcttgggtgacagagcaagaccctgtctcaaaaaaaaaagttatacacatgtaattattgcatgttccttcattattagttttaacatagacttgttaatctcaattagcttaattagcatttgagtttattgctaaaacttctttatgagttttaataatgaggcttggccgagtgtggtgggtcatacctgtaatcccagcatttgggaggccaaggcgggtagatcacttgaggtcaggagttcaagaccagcctggccaacatggttaaaccccatctctactaaaaatacaaaaaaattagctgggcatggtggcacatgcctataatcccagctgctcaggaggctgaggtggaagaatcgcttgaacccagggggcagaggttgcagtgagccgagatcgtgccattgcactctagcctgggcaacagagcaagatcgtctaacaacaacaaaaaaaaaaccaaggctgaatttcttgagtgattgagcagtggctatctattggacagtccagctgaacagtattttcctcaggctgggtgcagtaactcacacctataattccggcacattgggtggctgaggtgggcagatcacttgaggccaggagtgagaccagcctgggtaacgtgcctagactatatctctacaaaaaattttttaaattaagtggcccagtggccagctgtagtcccaactccttgggaggctgaggcaggggggtcacttgagctaggagcttaaggctgtggggagccatgattgcatcaccacgctccagcctgggtgacagagtgacctcgtctcaaggctgcagggagccatgattgcatcactgcactccagcctgggtgacagagagaggcctcgtctcaaggctgcggggagctgtgattgcatcactgcactctagcctgggtgacagagtgacctcgtctcaaggctagagggagccatgattgcatcactgcactccagcctgggtgacagagagagacctcgtctcaaggctgcagggagtcatgattgcatcgctgcactctagcctgggtgacagagaccttgtctcaaggctgcggggagtcatgattgcattgctgcactctagcctgggtgacagagagaccttgtctcaaggctgtggggagtcgtgattgcatcactgcactctagcctgggtgacagagagacctcgtctcaaggtatgtttcatgtatcttctcttttttcattgataaaggcccaaacttcccagagagaaaaacatacagccttaaggaatttggctagaagtctattcagggcatatgatacgatagaacagggattctgtagaacctggaagaaagtagtcactctaggagtaggttagcttgagagaagtagcaagaatgtacttaaagcagcagataatgagatagaattggggtaaattgcggtaggaatatgttagaagcaagggtggaactgtcgtcactgttacctcgatggcgaagccagaatgtgaggctcttgctcttagaactcacgtgagtaccatagcctcgcgttgtcttgcaggacacagattatccttatttgggtaccaccatggacacctataaggaactgattgagaggattcctgagttgaacaaagtggcacgagcagctgcagaggtcgctggtcagttcgtgattaaactaacccatgatgttgaattgaacctggactatgagaggtacaacagccaactgctttcatttgtgagggatctgaaccaatacagagcagacataaaggtgagcactgattccaattacgtttttattttgctgaatgtcaagtattttgaaatgtgatgtgttcctgtgtgttcctgttggaagggtgattgtagccatagtacattttaaagtgaactgaggtataattgtatgtagaattggtaacttgtttgagagaagtcgggaggctgtggattagagacctaggacagagctcagcaggtgtttcagaatccagagcagtgtcaggttttctgtcactcatgtctcccaggcagcccgtcagtaggacacggaatatgaagatctcagcaaggagttgggctgtgtgcctctcgggcgtgacccggatggaaagacagcacagctagcaggattccatctcgtagtgatctgcgcatctaaaagtcaaatattctattaaacgaaactgataagcagggtgaggtggtgcatacccgtagccccagctacttctgctgaggcaggaggattgctggagtccagcccaggcaacatagcaaaatcccatctctaaataaattaataaaactaatattaaagtagcttccagattgttttatggtactaggagttgatttttaacagatctcttaattgaagtaaatcactgacaaccgaatctttttatatcttttttatttttatttttattttattttttttgagagatgaagtctcgctcttgtcccccaggctggaatgcaatgacatgatctcggctcactgcaacctccacctcccggctttaagcgattctcctgcctcggctccccaggtagctgggattacaggcgtgtgccaccatgcccagatagtttttgtgtttttagtagaagccggggttttaccatgttggccaggctgaagtgcagtggcgagatctcggctcactgtaagatgcgcctcccgggttcacgccattctcctgcctaagcctcccgagtagctgggactacaggtgccggccaccacgcccggctaattttttgtatttttagtagagccggggttttaccatgttggccaggctggtcttgaactcctgacctcaggtgatccacccacctcggcctcccaaagtgctgggatcacaggcatgagccaccacgcccggcatcttatgtctttcttgaaactaattgtaactgttgaaaatggaacttaccaggtggaaataactaaatcctgaagtaccttgaaccaaaatgttttcccctatagggacattttcccccaaaaggagaattgaactggaaacaatatagtacataggattatataattatgttcaatttcttaatgagaatggttttcttacatgctgggctcaaatatgagtgtatatcacagtccatagagcttggaacccctgtgcaaggtgctttcggagtcttgagcttatctgcgagttccttttatcagaatcttacttaacgcacgttaaattagaaaggcatacaaaagaatgtccttagaaataaaacttctcatagcgaataatgtctgtttcaggaaatgggcctgagtttacagtggctgtattctgctcgtggagacttcttccgtgctacttccagactaacaacagatttcgggaatgctgagaaaacagacagatttgtcatgaagaaactcaatgatcgtgtcatgagagtaagtgaacttttgggaaaggaggaactaaagtatgtgtaaaataaccgataaatcttacacttctgcaaagtggacaaactctaggagtctagaattcctttaagaagggagcattaatggtttagctgtcattttctgtttctgctgtctaattcagaacttagtcaaacctagtctttttggaagagacttgctgtaaaacttccatgtatgctccaatggggaaaagaatctgaacacatttaaagttttcctttgtaaaatgaatcagtttcctttaaaaaaattttttttttttttgagacagagtttcactgttgttgcccaagctggagtgcaatggcacggtctcggctcactgcaccctccacctcccaggttcaagtgattctcctgccttagcctcccgagttgctgtgattacaggtgcccaacaccacgcccggctcattttttgtatttttagtagaaacggagtttcaccatgttagccaggccggtctcgaactcccaacctcaagtgatccacctgccttggcctcccaaagtgctggtattacaagcgtgagccgctgtgcctagcctcctttagaattttaaccttagaagattagcattagcctggttctcagcattcttttttccttactctgctatagaaagtctgatcagctggctgggtacagtggctcatgcctgtaattccagtactttgggaggccgaggcaagcggatcacctgaggtcaggagttcaagaccagcctgaccaacatggagaaaccccatctctactaaaaatacaaaaattagctgggcgtggtggtgcatgcctgtaattccagctgctcaggaggctgaggcaggagaattacttgaacccgggaggtggaggttgcagtgagctgagatcgcgccattgtactccagcctgggcaacaagagcgaaactctgtctcaacaacaacaaaaagccgggcacggtggctcacacctgtaatcccagcatgaattgcttgaactcgggaggtggagggtaccagtgagccgagatagcgctgttgcactccagtctgggcaacaagagcgaaactctgtgtcaaaaaaaaaaaaaaaaaaaaaaagtctgatcggcattcttaaatttgggacattttacatttgaagtgaactgttgttttactacaaaagtcacagggctgtgtaaattgccttgtgtgttgttttcgtaggtggagtatcacttcctctctccctacgtatctccaaaagagtctcctttccgacatgtcttctggggctccggctctcacacgctgccagctttactggagaacttgaaactgcgtaaacaaaataacggtgcttttaatgaaacgctgttcagaaaccagttggctctagctacttggactattcagggagctgcaaatgccctctctggtgacgtttgggacattgacaatgagttttaaatgtgatacccatagcttccatgagaacagcagggtagtctggtttctagacttgtgctgatcgtgctaaattttcagtagggctacaaaacctgatgttaaaattccatcccatcatcttggtactactagatgtctttaggcagcagcttttaatacagggtagataacctgtacttcaagttaaagtgaataaccacttaaaaaatgtccatgatggaatattcccctatctctagaattttaagtgctttgtaatgggaactgcctctttcctgttgttgttaatgaaaatgtcagaaaccagttatgtgaatgatctctctgaatcctaagggctggtctctgctgaaggttgtaagtggtcgcttactttgagtgatcctccaacttcatttgatgctaaataggagataccaggttgaaagaccttctccaaatgagatctaagcctttccataaggaatgtagctggtttcctcattcctgaaagaaacagttaactttcagaagagatgggcttgttttcttgccaatgaggtctgaaatggaggtccttctgctggataaaatgaggttcaactgttgattgcaggaataaggccttaatatgttaacctcagtgtcatttatgaaaagaggggaccagaagccaaagacttagtatattttcttttcctctgtcccttcccccataagcctccatttagttctttgttatttttgtttcttccaaagcacattgaaagagaaccagtttcaggtgtttagttgcagactcagtttgtcagactttaaagaataatatgctgccaaattttggccaaagtgttaatcttaggggagagctttctgtccttttggcactgagatatttattgtttatttatcagtgacagagttcactataaatggtgtttttttaatagaatataattatcggaagcagtgccttccataattatgacagttatactgtcggttttttttaaataaaagcagcatctgctaataaaacccaacagatactggaagttttgcatttatggtcaacacttaagggttttagaaaacagccgtcagccaaatgtaattgaataaagttgaagctaagatttagagatgaattaaatttaattaggggttgctaagaagcgagcactgaccagataagaatgctggttttcctaaatgcagtgaattgtgaccaagttataaatcaatgtcacttaaaggctgtggtagtactcctgcaaaattttatagctcagtttatccaaggtgtaactctaattcccattttgcaaaatttccagtacctttgtcacaatcctaacacattatcgggagcagtgtcttccataatgtataaagaacaaggtagtttttacctaccacagtgtctgtatcggagacagtgatctccatatgttacactaagggtgtaagtaattatcgggaacagtgtttcccataattttcttcatgcaatgacatcttcaaagcttgaagatcgttagtatctaacatgtatcccaactcctataattccctatcttttagttttagttgcagaaacattttgtggtcattaagcattgggtgggtaaattcaaccactgtaaaatgaaattactacaaaatttgaaatttagcttgggtttttgttacctttatggtttctccaggtcctctacttaatgagatagtagcatacatttataatgtttgctattgacaagtcattttaactttatcacattatttgcatgttacctcctataaacttagtgcggacaagttttaatccagaattgaccttttgacttaaagcagagggactttgtatagaaggtttgggggctgtggggaaggagagtcccctgaaggtctgacacgtctgcctacccattcgtggtgatcaattaaatgtaggtatgaataagttcgaagctccgtgagtgaaccatcattataaacgtgatgatcagctgtttgtcatagggcagttggaaacggcctcctagggaaaagttcatagggtctcttcaggttcttagtgtcacttacctagatttacagcctcacttgaatgtgtcactactcacagtctctttaatcttcagttttatctttaatctcctcttttatcttggactgacatttagcgtagctaagtgaaaaggtcatagctgagattcctggttcgggtgttacgcacacgtacttaaatgaaagcatgtggcatgttcatcgtataacacaatatgaatacagggcatgcattttgcagcagtgagtctcttcagaaaacccttttctacagttagggttgagttacttcctatcaagccagtacgtgctaacaggctcaatattcctgaatgaaatatcagactagtgacaagctcctggtcttgagatgtcttctcgttaaggagatgggccttttggaggtaaaggataaaatgaatgagttctgtcatgattcactattctagaacttgcatgacctttactgtgttagctctttgaatgttcttgaaattttagactttctttgtaaacaaatgatatgtccttatcattgtataaaagctgttatgtgcaacagtgtggagattccgtcgagataacttcgtataatgtatgctatacgaagttatatgcatggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtgtccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgattgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagcccctagcccgggcgccagtagcagcacccacgtccaccttctgtctagtaatgtccaacacctccctcagtccaaacactgctctgcatccatgtggctcccatttatacctgaagcacttgatggggcctcaatgttttactagagcccacccccctgcaactctgagaccctctggatttgtctgtcagtgcctcactggggcgttggataatttcttaaaaggtcaagttccctcagcagcattctctgagcagtctgaagatgtgtgcttttcacagttcaaatccatgtggctgtttcacccacctgcctggccttgggttatctatcaggacctagcctagaagcaggtgtgtggcacttaacacctaagctgagtgactaactgaacactcaagtggatgccatctttgtcacttcttgactgtgacacaagcaactcctgatgccaaagccctgcccacccctctcatgcccatatttggacatggtacaggtcctcactggccatggtctgtgaggtcctggtcctctttgacttcataattcctaggggccactagtatctataagaggaagagggtgctggctcccaggccacagcccacaaaattccacctgctcacaggttggctggctcgacccaggtggtgtcccctgctctgagccagctcccggccaagccagcaccatgggtacccccaagaagaagaggaaggtgcgtaccgatttaaattccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagtgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggtaaatataaaatttttaagtgtataatgtgttaaactactgattctaattgtttgtgtattttaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattgatctagataagtaatgatcataatcagccatatcacatctgtagaggttttacttgctttaaaaaacctcccacacctccccctgaacctgaaacataaaatgaatgcaattgttgttgttaaacctgccctagttgcggccaattccagctgagcgtgcctccgcaccattaccagttggtctggtgtcaaaaataataataaccgggcaggggggatctaagctctagataagtaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggaataacttcgtataatgtatgctatacgaagttatgctagtaactataacggtcctaaggtagcgagctagcgctggtttgcatttccctaaattagacagtaactttcagaagagatggaacttgttttcttgccagcaaggttgaaaataggtccttagctggaccaatttgtctgtttacaggaataagtaaggccttactggttaaccttggtgttctttatgatgagaccagaagccaaagacctcaagttttctctcctcttggcatctgccctatagtctttagttctttgtttttattttgtttttctttttttcccaacattttctgaaaaagaacaagttttagactcaatttgtcagacttgaaaagaacacactgccaagttttggccaaagtgttagtcttcaggaaagctttctatcattttggcactgagatatttattgtttatttatcagtgacagagttcactataaatagtgttgttttttttttatatatagaagataattatcggaagcagtgccttccataattatgacagttatactgtcgttttcttttaataaaagcagcatctgctaatgagacccacagatactggaagttttgcacttatggtcagcacttgcaactttaggaaggaagaatgccacaatccaaataatatgagctagaagaggattgggttaaataagagattcctaattgagttggggaaaaaaatgatagttttcctaagtgcagtgagttgtggccaagttaaatgtcatttaaaggctatggtagtacttacatctacaaaattttacagctcaatttattcaagatgtaactcaaaatcaattttgcaaaatttccagtacctttgtcacaaacttaactcacattatcgggagcagtgtcttccataatgcataaagaacaaggtagtttttgcctaccacagtgtctatatcggagacagtgatctccatatgttacactaagggtgtacgtaattatcgggaacagtgtttcccataattttcttcatgcaatgacatcttcagagcttgaagatcgttagtatctaacatgaattcctaacttcctgtagctccttagttttagttgcaaaaaacatttgtggtcattgagcatttggtgggtaaaatcaactgctgtaaaatcattacttccaagatccttttttaaaatgtgggtattttgttatctctggtttatggaataaaagcatacgtttataatgtttgctggtgacaaaggaatttaactatctccctttatttgcatgttacatgttatttcctaaatgtagttcttagaagtttgaattcatttttggtttttgggagggtagaaagtagagaataatgggtgttgggaagacaaagcttgcaaaaggatctttatgtacctgctcatcactatggtggctatgaattatgtaggtaatgagcaagatcacattaacaaagactagttaaccatcattacgcatctaatcagttttgccatggggtcagttcaaagctgccacctgagaatgtcactaggctctcagggtcttggcaccactcgcccaagttatatccaccagattattttgagcctcactgagttgttttgctactcagtcccttcagtcttcacagtgtttctcttgtaccagtatgagtatcaccgtgcataatgggattccaagtcttgaatgaaggcatgcattttacatagacgtctctgtgaaataacctttgttgtaagtggtgtttccagtcaaagcagtaagttggtaaggtttagtcttgggtgaaatgtggatctgagtaataagtagcttctgctacttatcaggttattgttggttaaaatgtagattttgaagataaaaatagatcttttgtcatgactcactgctagaatttgcatgacctttactgtgttagcttttgaatgccttttggtttggacttgccaacctatactgtctttgctgaaatgcccttataaatgtatacagctggtatgtaacaatgtgaagattccttacctgacttaataaaatacttcgataatactga

SEQ ID NO: 10 - DNA, 32523 bp, synthetic construct
  source 1..32523: mol_type = other DNA
cagagggccggccggggggtggggccgagctataagctttgggtgggaggcagcgctgccttcagaaggcgtgcggagcgcgggctgctgcattgcggactgtagaggcgcttcctagtgagtgactcccttgtcagcggcacggcccatcgtggtcctcgcgtggcgggcggaccagagcgagacgccagggcctgggtggtgcgggcggggaggcggaggggtgtcgcggagtccggggctgaggagcgcgggttgcaggtgcagcgcggtgggtgtggggagccgctgtaccctgcgcccctcgggtcctccgggccttcgcaggccagtgctaggccgcgggttcgagagtcaccacgctgaggcgcaggcttgttccgccgggagcacgtggtggcggctggaggaagtcgccccagggaacggctgtcggggtacgtgggtgaccttggggcccctcgcaggagggcgtcacagctgaaaaggacaaagctgttttctattcggttactagtgtcacggacatttagaggggcggggggagcttccaataactgcacgttggaacttcggcaccacctggtcggtttttttgccagtctctccctcttggcccagcgtgtggaatctcatttttctagggcagaataggtctgaacgctgcaggtaatactaagaacgtctctagcatctcctaagatgggagaacgtagaaatacgacctctttgtacgagctcttttagaactagctgtagagaaccagcgtgcaccctggtgttggacagctctctaaaatggtgtttgagggtaagaaaactgcatttgcaaatttttcagttagcacactttgtcccgagcgtaaaatgaaatgatctccttatacttaggcagaagactggactggattgctgttcagtttctgtgctatttttttaaaataggatttaagtgggaaatagttgtgttacagaaattctcggctattctgtgctatttttttttaaataggatttaagtgggaaatagttgtgttacagaaattctcggctacctgatacttttattctaagattagatgagttggctctgagctgtgaaatatgacctctttgacaaagacatttaagctgattcaggatgttatctacaaagaaaacgggatttagcttgtgtgggtccacttgcatttattttctgttaaggattataataaactgctttatacaggaatccaatatcagctgttttttatatagagagcataatactttttactttgagagaggatgttgtcaaggaatggtggctgtgaacctggcctgtgttgactggttagatctgtctgcctaaccccaccctaaggctaagtagttatatgcttgtggcaatgtgcttatttataatagggcaagattatgggctaaatttgggttagacaacaatgaaagttaattaaacgaccctcaggccttgggtctactatgtgtaagtgatttccttctctcccagatgagtgctatacaaaataaacttcagtgacctcaagtggttttgaccttttggttgctattcagaaaactatggaaatgaaaacctgctacctatttcctattgccttttcaatttcccaaagaaggtctcccctatgaatccatgggtagccttgaactcagatccacctgcttctgtttggagagtgataagattaaaggcaagtgccaccacacccagcaaagtaggctcttaaaactaaaacctttgcagtcgggcatggtgccacacacctttggtcccagcactggggggtaaggcagaggcaggtggatcactgagtttgaggtcagctagtgctgaagagtaagagcctgtctttaaaacatctcaacagctgggcagtcgtggtccattcctttaatcccagcacttgggaggcagaggcaagtggatttttgagttcgaggccagcctggtctacagagtgagttccaggacagccagggatacacagagaaaccctgtctcaaaaaaccaaaatataaataaataaataaagattgggagagaagtcaaggatctcataggtggtcagggagctacaactgcagtagtaaagaagtaggacttaaaagaacagggccggcactaattttgaggatctagatcgggccctcaattaaggaacttccttttgtgtgaactcagaatttgaaatgaaatgtgcttgtcagaaccattgcatggcttattttttaatgaaaagtctggctagtatctgcttatcttccagcttccagctcaaagttaaggtcatagatcaaaagaactatgtctttatcttagttgtatcttaatttttattagaattgaatggttttcctaatgtttggtaacatcaaaggtgtgtaagtaaaagtgagaaatcaagattaactttctcttggcaaagattgttgacattggtgacatcttggaccaaatgagaattgttttacttttaaatgtcccatcaacagctctcagttaggctgttctatctggtttgtcttgccatgcttgcagagtatagatttgacaatttgaaaattcaaaaagctatataaataggtatgttgctatatgtaagattttaaatgagtcagttaagacttaaagaataactgggtttattttatcttgtcaggttatcactgtgtagaccaggttgaccttgaaaacaaattctctgcttcccaagtgctgggattaaaggcgtgtgtcactagctctgacactggctactttggaactactatggtgttcacaaatgcagagttgagtgttgggattaaatggaaatttcatgtctttttttttaactttcccttctacacagggcttctctgtgtagtcctggctgtccttgtagctctagactaggctgaattcaaactcagatccacccacctaagtgctgagattaaaggcatgtgccaccactgcccagttctgaattgttggggttttttttttgttgttattcttaattttagttcgatgaattaaaatcgaaataacttgtttcttagaaaaataaggtgtaattgggttataaagccaaatttagacattaataccaacagcctgtttaggctcaaaattgttcaatcattttattagtattattattaatcatatcaacttgagacctgtttgggaaagcagaatattttagggatagctatttcagacaagcattaatgtgttagctgtttttttccccctagaatatgattaaaattggctcagggtggggccttctagttctggctctagcgattgggtctgtttctggtgaggtggtagtgataaactgtaacagaagggacaagagattgggcttctgagaacatgtatgatctggtatgtgactttaatcattaaagcatggggattcaaaaatactaatgaataggccttagaactagtcctgagtgttttgtaaaataacagtcttaattctcctagtttctggatttttttctttgtctttggatactaagtttaagcatttatttggacagagtggtgcccactagctgttctattctagtacttgggaggcaagaggcagaacgaacatggccagcttggacaacttaagaaaactgaaaggcttgccatcctagtttttcttttgattaaacacacttatattctaactagttttctctatcctttgggttttgttttgttttttatttgttttgtagctctagctggctggcttcggaattgcctgcctcccaagtgctaggattaaaggcatcacagtcactacccaattgatatcatgattcttaattcaacttctaagaacaaattatcacactctgaatctaacatggaatagcattatccatgttcagatatctttgtctcaaggctcaggtttatcctttgtagctttctttttgctatcccacctctattcagcatccagtcaaggatcactgagttggttatcagtaaattaaacattttaattaatgtctaggagacaggttatggtatagttctcagtgctgggaagttgacatggtaggatctcagttcatggccagccagaactttctggtgagatcctggttcaagcactacagagcttttctagaaaagtaactatatttaggagtaagtttgatataatgacaatcccatcgtaagccttcagtaacctgatgcattggtctctgttttaatatcaggtactcccttgtagcagctgagaatgatggatcaagccagatcagcattctctaacttggtaaggtactttcatctatctgaaaaaatgttcagtaaaaaacaaaacaaaaacatgacttcacccagtgagtacaagatgatacccccaattttttttttttttattatatgtatgtacactgtagctgtcttcagacacaccagaagagggcgtcagatctcatcagatggttgtgagccaccatgtggttgctgggacttgaacactggacctttggaaaagcagttgggtactcttacccactgagctatcacaccagccccaataccccaaattttgaccagccttttttttttttttttttttttttttaaatctgaactaatggtattaaggtgaaacagattgcacaaaagaggtactaggtttttctttgaggcaaggtctttctatattgcctcaggttgaccttaaactccaacttcctttttttccccaagtgctggagattgtagacagatatatacaacaccccaaaacaaatatgtttagtttcgattaagattcattatgtggggctagagggatgacttagaggttaagaacactgcctgctcttccagaggtcctgagttcaattcccagcaaccacgtggtggctcacaaccatctgtaatgggatccgatgccctctttctggtgtgtttgaagacagctacagtgtcctcatacataaaaaaaataaacaaacagatcttttaaaaaaaaaggtacagtgtacttatacattatataaatgaatgattctttaaaaaattaattatgggaaatacttatgaagaatagggtagctttggctgttttggaaacgttatataacaaggtagaactaaaatgtatgccagtaatcccagaggaatcattagccagtcagggctagtctgagcaatgtggcaagataaacccatctctttaaaaaaaaaaaaaagtattataaatagaaatgttataggaaacaggaaatagaaaccctcgaaaggctgaatgaaagagtattagtgggctggagaggagagactgactgagaagaccagtgtgaactagcccttgactgggcccagggaagaatgttggtttgaaaggagaacaccctaaggaatatatagcaagttatagtaatggcccaataacaagcatatacctaaacgagcttttcctaaacattcccgtgcctgttcttcatcatgcctttttccgcaacacagtttggtggagaaccattgtcatatacccggttcagcctggctcggcaagtagatggcgataacagtcatgtggagatgaaacttgctgtagatgaagaagaaaatgctgacaataacacaaaggccaatgtcacaaaaccaaaaaggtgtagtggaagtatctgctatgggactattgctgtgatcgtctttttcttgattggtgagaatgaccattccaaacttcaatgttttctataaccaactctgggaagtctgttatgtcagctcagcatatagttattttgtaccttattttttttttttttttttttggagacagaggcttactctcttgcctaggctagagtgcagtggcaccatgttggctcactgcaacctcagcctcccaggtttaagcgattctctgccttagcctcctgagtagctgggactataggcgcccacgaccacacctggctaatttttttttttttttttttgagttggagtctcactctgtggctcaggctggagtgcagtggcgtgatttcggctcactgcaatctccgcctcccaggttcaagcgattcttctgcctcagcctcctgaggagctgggactacaggcgtgcaccaccacacctggctaatttttgtatttttagtagagacggggtttcaccatgttggtcaggctggtctctaactcctgacctcgtgatctgcccgcctcagcctcccagagtgctgggattacaggcatgagccactttcccgtccactttatgcctttttaagtgccactaggaagacaatcatgagtagattctatacctgtcattgaatttaatctggttggaaagagagacccatgaattatttcaatacgacgtgcaagtgttttctttttatttatgtatttttttgtttgctcccactccaccaggaagcaaatgctacgctagaaaatatccgcaggacactggcggtgcagcagctcagtctcatgtttcaagggggtcttttggaaaaaactacctatgttttgaataggtagctatttgaaataaagctagtatataatttattaagtggtttaaagtatacaaaactgagatgcattatatactttaaaactacttagaatttgatgaaaaccagtattttcaaggcttacctgaaaatattaacagattaacagggatagattacaaatgaaattctgagttcagctctggaactttgtctctttaggatttatgattggctacttgggctattgtaaaggggtagaaccaaaaactgagtgtgagagactggcaggaaccgagtctccagtgagggaggagccaggagaggacttccctgcagcacgtcgcttatattgggatgacctgaagagaaagttgtcggagaaactggacagcacagacttcaccggcaccatcaagtgagtgccagctgctgtgcaagtatctagacaagtaattcaagaattatgataggccacggggaaacaataatggtgacactgtggggaatggcttgttagagaagacaagacttgtcatgtttagctaaagaggggaaggggcctgtaaaaaactgaaactatactggtaggggaaaaggtagaacttctctggggagtctcctttttcttaaaaaattaataggcccttgatgttttccccatctttgtagcttattgcttgctgtcattctctggctctgatttttcttcttaacctcttgtaacgttgtactaagctctgctttgctgacaaggacatcagtagcttttctctgttttaggaagcaacacatgccagaagtagcctttttcatctccttgatcatgggtaactgcctgccttaagtctctagtgcttcttaatttctgtgatactcacccattaacccctaggccagtcagtatccagagacaatgtctccctctggtggaggctggaatacggtggcgtgctctcatctcaccgcagcttcatccccctaccatagcctcctgagtagctgggactataagcacgcagcaccacacccagctaatttttttttttttttttttaagagatggagtctcaccatgtctcccaggccggtctgttcttagatttttatttatttacttatttattttgagatggagtcttacgtgttgcccaggctggagtgcagtggcacgatttcagctcagtgcaacctctgtctcccaggtttaagcaattctcctacctcagtctcctgagtagctgagatcacaggcttgtaccaccatgctctgctaatttttgtgggttttttttttttttttttttttttgcagaaacgggatttcaccatgttggccaggctggtgtctaactcctgacctcagatgatcctcctgtcttggctcccaaagtgctggaattacaggatgaggcactgcacctggccaaatttctattttatatttatttatttattttatttgtaatgggctttctggcaaaaactagaaagcatgctagacaaattctaaaagagctgtaacacttgtccatagagttttactatctttgcacatacatgcctctgtgcaaagaataaatttatcttttcctttttgcaagcttcctacacttacagtggtactataattctttagtcttaagttatggcattttctacttcatctagttgatgcttttgtgtttatttactgtacaccagaccttatggttaaagtgagagtatagtccaccccacaaaaatgtaagaactaatatgctgagtaaatagaaagaagtatgggagatcggaccaaggagcagttctgcttcagggtattcagatgcttcacaagtaggtgagaattgaaactatgcactctgttcagcacagtattactatcttagattttatagtagatctggaagctactcatttcccctaaaactatcctaagcagatacattgtgttatgatgaggctcttgaaggagtgctttgtagtgggtaaagaagctttgaagttacaagatttggggcaaaattttttttcttttgtgatattctggtcagtctagaagttttgggtcacagttcatttatctgaactatgagggataatattttgctttcaaaacaatttgtttaatccttgacttaaatgtttattttaaagtaaacttaagttcatttaaaaggcattttctcctccaggctgctgaatgaaaattcatatgtccctcgtgaggctggatctcaaaaagatgaaaatcttgcgttgtatgttgaaaatcaatttcgtgaatttaaactcagcaaagtctggcgtgatcaacattttgttaagattcaggtcaaagacaggtatgttgaaagatggtaaacttatttttatacaagtagctattttcaggtgtgctaaatatagcaaagactttttgttagtgttgttggcttttttttgaaatggagtctcgctctgttgcccaggctggagtgcagtggcacaatctctgctcactgcagcctttgccttctgggttcaagtgattctcctgccttagcctccagagcagctgggattacaggcgcacgccaccacgcccagctaatttttgtatttttagtagaggtgggatggtctcgaacttttgacctcaggtgatcagcccgcctgggcctcccaaagtgctgggattacaggcgtgagccaccgcatgcggcccaaaggcttttaaaataaaaatcaagcttaagatttagaggtaaatttcctcaagccaaataatgcaagacatactaaatgtaagctattgtgttttttggaaggatgaccttaggcttattttaacttaatctttttaacagcgctcaaaactcggtgatcatagttgataagaacggtagacttgtttacctggtggagaatcctgggggttatgtggcgtatagtaaggctgcaacagttactgtaagtaaagcaaaacagtgcatgagactcttccctattgaatcattcaaaactcatcttttctgttcttaggagttataaatttacctgtaaaatgtaaatgatcatgagatattttggttttcaaccctctaatgacacagtcaacatgcattgtcttctctctctaatcactttccccatgtcctgttttattttttctttatagtactgctagcagctgacattatctatgcctttgtttccattagaatgtgagccagatgaagaatcatgggttagttctatttactgccatgttgcagagctttggagaatgcctgacatacatagtagtatttgctaaacaaatgcatatcctccctgtggggacagatgtaaatatccatcttggctggggcctagtcttagcttgattcgttaatgcttgatctttttctatttttgttttgagacagggtgtcactctgttgccctagctggagtgcagtggcgtgagcttggctcactgcattctccaccaccacccaggctcaagtgagcctcttgagtagctgagatgtccagttaatttttattttttattttattattattattattattattattattattatttggtagagacagggttttgccatgttggccaggctggtcttcaattgaaactcctgggctcaggtgattcaccaccctcagcctcccaaagtgctgggattacaggaggaagctgcttcgcccagcccttgatctttttaaatttaggcaaatatagtcaaattaacacagaaatagagaacagatagatagaataattttcagcttaaaaatcacatttgtggctgggcgtggtggctcacgcctgtaatcccagcactttgggagtccaaggtgggtggatcacctaaggtcagagctaccagcctggccaacatggtgaactctattaaaaatacaaaaattagccaggcgtggtggtgggtgcctgtaatcccagctacttgggaggctggggcaggagaatcgcttgaagccaggaggtggaggttgcagtgagcggatattgcgccattgcactccagcctgagcaacaaaagcgaaattccatctcaaaaaaaaaaaaaaaatcacatttgtaagtcaggcgtatctctagtggatacctttcggggctggtgcagttctgtgttgattccttgcctttctggatacttgtgctttatccctttgcctggctgcctataagccaggagtttagagatgggtaggttgttctgttaaagaacattgaagatttttgttcaacttcaagctttctgtattgagggcttggttgttagtgtcagtttatggctgttcatctggactcttatgagcttaggaacatcatgacacatctaaagttggtgtatgcaagtcactttgttgtaatagatggtgttttaattttgggacagatttattatcagacctttcagttaaaacctatttctatgatgtcctcaaggctaagattgttccaggcctccagtcccatgaccagcctacataggcctctttaatctaaatgcccttcagatgaaaggcagcatgggataaaagcatgaaagacataggaggccaaaaatcttgattttattccaatagctctgacctaaaagttgtaaagttttttagaactcatgtcttttttaggccttggcctccttttatctcttacttgggttgcctgtcttaggtttgatctataacactcttctgtctcaagattttagagctgttccgataccataatgctcattaaatctaatgtctttgttctagggtaaactggtccatgctaattttggtactaaaaaagattttgaggatttatacactcctgtgaatggatctatagtgattgtcagagcagggaaaatcacctttgcagaaaaggtgagtatgagttattataatattaaatacagtacttttggtatcttactcttcaggtaagtgatatttcttgttaatattcttacattctgaaaatcagcaatcttaaactctcataccagataatataatttattaaaatgctcaacaaatgaagtaagttccacttacctaatcagaagtgcttaagtgaaagctatttctctgtataactcagttttacgcatatacagaattcagctaaaagcatgttttataaaaaggaaaattaatagccaaacatacacaaggaactagagtggaacatatcaaaagacagtggtgatttctgcatttttaaaaaaagtggcagggctcacacctgtaatcccaacactttaggaggctgaggtgggaggactgcttgagccctggaggttgtgagatgtgactcgtgccactccagcctgagtgacagtgagactcaaaaaagtggttgtcttagatgggtttttgttatttttcttttcttttttttttttttttttgagacggagtctcttgtcaccctggctggagtgcagtggcgtgatctcagctcactgcaacctccgcctcccaggttcaagtgatgctcctgcctcagccttctgagtagctgggattacaggtgcccaccaccaccatgcctggctaatttttgcatttttagtagagacagggtttcacgacgttggcctggctggtcttgaactcctgacgtcaggtgatccacctgccttggcctcccagattgctgggattacaggtgtgagccaccactcctggcctttgttattctttttttttttttttttttttttttgagagggagttttgctcttttgtccaggctggagtgcagtggcgcaattttggctcactgcaacctctgccttatggtttcaagcgattctcctgcctcagcttcccaaatagctgggaatacaggcgccagccactgcgcccagttaatttttgtatttttagtagagacagggtttcaccatgttggccaggctggtctcgaactcctgagcttgtgatccccctgcctcagccttgcaaggtgttgggattacaggcgtgagccaccactcctggcccgttactcttattttgcaaacaagtgagttaatttttcaggaagcaattaataaattaccagataaacaatttgaaatatttggaagtattataagtttagtcctctgtaaccatttggcctttagcataatgtccaggatgtattctcatttcgtatgatcagatcttcattttctgagctttatatgttttcttttaggttgcaaatgctgaaagcttaaatgcaattggtgtgttgatatacatggaccagactaaatttcccattgttaacgcagaactttcattctttggacatgtgagttatttcttgagtaaatcaccgttttgagttccttgagttgttcttggattcctgtattagcagaaatagcactgtgtcttccttaactgctcttttttctgggagggcgttagcagtaagcaagaagatagattacattgactttgaggctttattatttgttgctaaaagtactttgtcaatagtgccttgaatttgagaatttccatatgccatgaaaacaggagtcacattgtagagccactcagatttttgaggcttcaagttggtaaactaaggttgggctgcagccttataccaaacctgaatcttacagagaagttttgaggaagtatgtgatggagtgaattgctcttttgttttcctaggctcatctggggacaggtgacccttacacacctggattcccttccttcaatcacactcagtttccaccatctcggtcatcaggattgcctaatatacctgtccagacaatctccagagctgctgcagaaaagctgtttgggtaagtttttatttgaaagcggtcttgcatagtgaggttttatgattagggaagaacggtaatatgttgattaaaatgttaaacatgatgataattttccacatttgggaatttggaggatggttagctgttaccttggtacagatataaatgaatttttctctataaagagatcttaataatggactttggacttataaacacaggagaataaggagacttataaacccagtgaattccttgtgcaagattcctggcatagcttctgctcaagtacttctgtgctgaagcacgatgcctatagcagcccattcagttagtagagagaatctgttagaattcatattgggataaaatcctattccccgaaacatccatttgtctggatttttcttctaagcaatacggtatgattccagttaccttgtctatagattgagaagccttgcttcttatgatggtttccagagaaaccatttatggttgtggctcgctcctctggatatcctccctgtaacgttagtgtctctctttaaaaacagagcccagttccattagagtacgtagcattaattttgatttggatgtggacatatttatcatttcctgttattacaggatggagatctagtttcaggcctttatgaaagcatttatctcctgtatgacataggttctctgatcctgtttctttaacataattggatagtgaaaatatattctacttgatagtctcaaatgaagaacatctgtatataaggagtatatgaaataccatgactgttgatcatgctgagaggcctttagcataggggagtgtgtaacataccatgactattgataacgctaagaggcatttagcataggtgatttttttgactcattctacctaccacctacaaatcgtaaccaacctgtgaagccagcgtcccatcttatccttatgtagtaggggagtaataatgtttcaacctatatttatgtaagtaagcccccttaggagcagctattatttgggtaacacagaggaattagataggggaagcacaagattttttttttattttgacagtctcactctgttgcccaggcaggagtgcagtggcatgatcctggctcactgcaacctccacctcctgggttcaaggaattctgcctcggccttccaaagtgctgggattacaggcgtgagccaccgcacccagctgaggaatattttttataactgagctaagaatgtgtactatccttgttagtggtgacagttgggaaacataaaagtgtattaatattcttttatatattagaagaacttcattttgagtccatcttggtatgtattccaaatataaactacgtaagtatgtctggcagaaaggcgcatagttagagaagtgttttaaaatattgcttaattaatggtttccaattggctgcctgcagatcaaagtaagacgcaaatggttcaccactagagagtaagtttttttttgtttttattttgttttgttttgttttgtttttttttgagacaggacttgtgctctgttgcccaggctggagtgcagtggcgtgatctcggctcactgcagcctccgcctcccaggttcaagcgattctcctcctcatcctccttagtagctgggattacaggcgcatgccaccatgcccagttaatttttgtatttttagtagagtcggggtttcaccatgtcggtcaggctggtcttgaactcctgaccttgtgatccacctgcctcagcctcccaaagtgctgggattacaggggtgagccacttcgccgggctgagagtaagttttgtttatatgtcctcttaatctgtaacttcactggacaggagtaaaccctgggcaaggaacaataactcagaacttacgcctgctttctgattctaggaatatggaaggagactgtccctctgactggaaaacagactctacatgtaggatggtaacctcagaaagcaagaatgtgaagctcactgtgagcaatgtgctgaaagagataaaaattcttaacatctttggagttattaaaggctttgtagaaccaggtaaagaccgcccccccccccccccgccccgctttttttttgttttcttttctgttcctaaggatgtggctagagaaggagcgagtgtaggaatgctggcttggcttggttttatgaagtgctcaatcttgtctgtcctaaagttaattgtttatgtgttagtttcttttttttttttttgagaaagagtttcactcttgttgcccaggctggagtgcagtggtgtgatctcggctcactgcaatttccgcctcccaggttcaagccattctcctgcctcagcctccccagtagctgggattacaggcatgcgccaccacgcctggctaattttgtatttttagtagagacagggtttctccatgttggtcaggctccatgaggtcagagctcctgacctcaggtgatctgcctccctcagcctcccaaagtgctgggattacaggcatgagccaccgcgcccagcctatgtgtcagttttattgggaggtaataaggccctaggaaattcttgttaaaaattgaccattatgactagaaaatctattctaagttattgactagaccagtgataagcaaatttcttaagggggcagataataaaccttttgccaggctatacagtctctcttttagctgttcaactctgtgggagcaagaaaacaggcacagactgtgcgtagtgaatgggcatgcgggctgtagtttgccagtccctggagttaactttaaatgcaatcaaacagccaacacttactaagaaataagtccttggacttaagtagtcagtaaatttaattagtagtgtaggaaaaaagtagctctactagtaaggtaaaattaatattctcatgtgaatttttaattcccagagtttttagtcagatttgaagtaaggctcttttatccttaatacatatactgtgcttttacctttctactgatgtgctaattctagaaaagtttagaagctgattatataagttctttcttcctcttttttcttttttaaagatcactatgttgtagttggggcccagagagatgcatggggccctggagctgcaaaatccggtgtaggcacagctctcctattgaaacttgcccagatgttctcagatatggtcttaaaaggtagagtacaaattttgattcttttgaatattggtgcactgcatacagttctagatgttatactgtgctttgctcactttgcctgcattcctgtggttctcatgctagactctagttcttaaatacaaggcagattgtcttttgttgtagctgccttttcctttgaaacagctttccataaagtgtcttaactgtgctagaaatcaccagtttctttgagacagtggagttactgagccctagtgcttagtgtggtggatccagagtgataaggtggatcatagtccctaagcaatctatttgaaagcagtagcatacctctctactcatgtactgaggcctaacagctattgaaattttgttctgtttattcatttattcattttttgagacagagtttcactctgttgcccaggctggagtgcagtggtgtgatctcacttagctcactgcaacctctgcctcccgacttcaagcaattctcctgcctcagcctcctgagtagctgggattacaggcacccacaaccacacccggccaatttttgtatttttactagagacagggtttcgccatgttggccaggctagtctcctgacctcaagtgattcacccgcctcagcctcccaaaatgctgctagtgagccaccgtgccctgcctcttttgtttatttttgagcctttctgtcctactttccactctttcacactcctacccacccacacattcaaaatcacgtcacattcttgaatctttgggtcttagggcaaactgcaatgcttaactttaatacagagtacattaatggagaaaaagatacagtgagggaagtgggagggatggagggcagtgtgatacatgtaacctcaaaaggtgctgtcacaaatatagtttctctgcaaactcttcttgtaacttaaatttgtgaatttttattttattttagaattatttttttaaaaatccaatgtttggatttacctctgaagatttttctcaagatgtgcatcatcatgcttacactgttttcaatgatctctgggtccagaagttaaggaactccaagaatgaaattgtgaaagggtgactactggcccctgctatgtactcaaaatcttatttgcagatctcctatcttgctctttgtaggttactttaaaaaaaattattcctattcttttattcttttacctggggtctcaaaaaaagattttgttagttttttttttcttttttggctcagaaaaaaaagattttggaatctgctatcttggaattagaccttctgattcatcttaagtgggataccttccatccttgtctgtgtataacctttcctcaggtaaagctaacttttttctctttcagatgggtttcagcccagcagaagcattatctttgccagttggagtgctggagactttggatcggttggtgccactgaatggctagaggtattctttatcatcccttcccatattggacacgagcttgtgggcttaggctgttgtccagaagtgatgtttttataggtttgattttaccacttttgcctttgcgtttagtctcagtagagtccagaattgaaaatgaatccctaatgctactgtatgtgataaataaacagatttatacttattagtgttttcccttctcttctagggatacctttcgtccctgcatttaaaggctttcacttatattaatctggataaagcggttcttggtaagtatccctttcattagctgtgtatgaattcaggtaaacttttttgagatggagttttgctcttgtcgcccaggctggagtgcaatggcacgatctcggctcactgcaacctctgtctcccagattcaagcgattctcctgccttagcctcctgagtagctgggattacaggtgctcactaccacacccagctaattttttgtatttttagtagagacagggcttcactatgttggccaggatggtctcgaactcctgacctcaggcgatctgcctgcctcggcctgggattacagatgtgggccactgtgcctggccagataaactactttgaagtggaagaaagcttttttttttttttgagacagggtctcactgtattgcccaggctagagtgcagtggcacaatcttggctcactgcagccttgacctcctgggctcaggtgatcctcccacttcagccacctggctaattttttttgtagagatggggttttgccatgttgcccaggctggtgtcaaactcctgagttcaagcattctatgtcagctgcccaaaatgctgcggttacaggcatgagccattgccctcagcctgtatcttaaccttcctttaaatagtctgtcaagttacacagtgagcacaattgcttgtctagaacagtgggtagttctcagtgtggcccccagatgagtagcattaggaactgttacgaatgcaaactgtcatgtctaccccagacctttgagtcagaaatgggagtgttggtctaaaaactgggctttttgggctgggcgcagtggtgcacgcctgtaatcccagcacttgggaggccaaggcaggcggatcacctgaggtcaggagttcgagaccagcctggccaacatggtgaaaccccgtctctactaaaaataccaaaattagccgggtgtggtggcgagtacctgtaatcccagctactcaggaggctgaggtaggagaatcacttgaacccgggaggcagaggttgcagtgagccaagatggcgccattgccctccagcctgggcgacagagcgagactccatctcaaaaaaacaaacaaaaaaactgggctttttttttggcaacccttggggaataaaaacctattattttcttaaggacaaagtatcctgaagcaaaaaaacccaaacaaagaaacaaaaaactttaacaagcactacaggtaattctgatggactaaagttttaggaccataagtctggactatattgaggtgagaagaaactaaactatgccatatagaatggtacttagagagtaattcacatcctgttacgttgtggcatcactgatagaaatattggataatgaaacttctagaagagtttgaatgttcactggcagcagaaaattgacaaaagggtttgaatgttatttaaagtgcagctgtacattcaacaggaatggggtaaaaaagaaagtgcagatgtagctgatgagctgagaatagtgaaatgtctacatgggggaaaaaaggaaagagttacaattaaacctctctaggttagttatttccctgttgtatgtttgccgcagaatgtgctgagtatagcaagcatactatgtatagctctaacctgggtgaaccagaaagttaaacatgaaattgctaatgggggaggctgcacatgtgtgggggcagtggatttatgggacctctaggtacctttctcttaattttgctgctgaacctaaaactggtctgatttttttttttttttttttttttttttttgagatggagtctcgctttgtcacccaggctggagtgcagtggcgccatcccagctcactgcaagctccgcctcccgggttcatgccattctcctgcctcagcctcccgagtagctgggactacaggtgcccgccaccacacccggctaattttttgtatttttagtagagacggggtttcactgtgccagccaagatggtctcgatctcctgaccttgtgatccacccgtctgggcctcccaaagtcctgggattacaggcgtgagccaccgtgcccggcctaactgctctgatatttttaaaaaaggtgacttggattaaagtatgcaaactcaaggagtagtacgagcccacttgagtgaagttagcttagttgctaaagagcttgataccaaaattacttttgtttacttgatgaatgggatagttgttgcacagggccactgatctagattactgtcttatttgtcaagtacttatagttgtagaagtagcagtgtgaaaattatcggaatggagaaggggcaatgataaaacaatttattttcaggtaccagcaacttcaaggtttctgccagcccactgttgtatacgcttattgagaaaacaatgcaaaatgtgagtatatacctcattacaaaaatgtatgacttaatttttgttgaatcaacctgagataaaaaacactgatatgtaaaccgtagtcagtaacaaaaaataggaattgagaataaattttatagcagcgttatttaagggatacttgcctattgaaccatatgagatgagggctccaatcttaaagaatatgttgtttatttaggaatatataacaaaatgccatgaggcctaagctgtagtgcaggggcccacaggggattgtctatagagtcacaatgctaaggaaggcttaaatcaacttgaactatactttgagaaggccggaggaaatattcattgaataaacaattaccatagttttgaatgagggagagcatgtgaaagaaaatatttgcctatgtggggtggggggaggggagagggatagcattgggagatatacctaacgctagatgacgagttagtgggtgcagtgcaccagcatggcacatgtatacatatgtaactaacctgcacaatgtgcacatgtaccctaaaatttaaagtataattaaaaaaaaaataaataaataaataaaaaagaaaatatttgcctaatgggattagaggctgtatattggagtgagtaaagttagaaacaggtcagatagcagacattggccttgatccaagagatactagggattataggatctcaagcaaggtgatcatgaatttgccagttaggttggtggcataagatgcactagaaggaagaaaccagaaacaagataggcatagtgtgaagcccagagagggcttttacaattgtgggtaagccaccatggttttgtttgtttgtttgtttgtttttgagatggagtcttcctctgtcttcctccagcactcaggctggagtgcaatggtgcgatcttggctcactgcagcctccacctcctgggttcaagcgattctcctgcctcagcctcctgaggagctgggactacaggtgcacaccaccacacctggctaatttttgtatttttagtagagacggggtttcaccatgttggccaggatggtctcaatcccttgacctcatgatctcttgaccttggcctcccaaaatgctgggattacaggtgtgagccaccgcgcccagcttttattcatttttttttccttttttttttttaagagataaagtcttgctgtgttgcccaggctagtctcaaacttctgggctcaagtgatcctcccagctcggcctcccaaagtgttgggattgcagttgtgaactaccccacccggcctaggcccacttttaaaatgttaattaagaagaaaacattttttcctcaccataactaaaggaactgacaacatgagcctagttagtaataacttaaagttgagattgttttaactatcataaataatttcttttcaagaagatataatacaagttttttatttaaaaattgttatagagtaacatgttagtgactagcctgaataaataagttccaaaaggttagttttaatgataatttcttatcttacaggtgaagcatccggttactgggcaatttctatatcaggacagcaactgggccagcaaagtgtaagttgagaaaagtgaatgaacaaactaatagaaaagcagagattctacctactacattaggtagaatctaaatctgtccttgcattgaacttacttacacctaaagatattcagctaaagaatttattttggatggggggactagcacgatagaacagtctattctttaaaacactttctaaagacacattctttgtctttgcagtgagaaactcactttagacaatgctgctttccctttccttgcatattctggaatcccagcagtttctttctgtttttgcgaggtaagtctgttcatttaaatgacaaatggagagaggctctctaaaaggaagtgatctgtatttgggaatagggcattgcaatgggaatgcctgtgctatagtcaactatgtacatattcagaggagtaaaggaaaacaagtttttaaggacaaatgacgaggattacaaaattactttgaggtaattattcttggctaccaagatcaataacaaggatgacaccagtccaaggttgacaggcagttgctaggcagattttctcacgagagaagttttttggtgcaaggttgcagtggcctttgtgtaaggttgtagtttttgtaaaaaggaaaaaaaaaaacccttaatccttgttatcagacatacaagggtgagacccttctcttcagggttgcatttttgttaacactagtgactccattttgattttgacaacttgcacatatcttagttgttgggttttttttgtttgttttttgttttgtttttttttttttttttgacaatggtgacttgctctgtcacccaggctggggtgcagtggcccgatcatggctcactgcagcctcaagcagtcctcccccttcagcctccaaactgtttggattataggcaagagctacttactacacctggccatggatcttcttgtgtcaactttgagaccaaatttactgcagtgcttaaaatgttttaagaagttatacacatgtgctggatgtggtagctcacgcctgtaatcctgtagcaggacaagccgcagacaaatcccctcagacaccgagttaaagaaggaagggctttatttggctgggagctttggcaagactcacgtctccaaaaactgagctccccgagtgagcaattcctgacctttttaagggcttacaactaagggagtctgcgtgagagggtcgtgatcaattgggcaagcagggggtacgtgactgggggttgcatgcaccggtaattagaacagaacagaacaggacgggattttcacagtgcttttctatacaatgtctggaatctatagataacataactggttaggtcagggctcgatctttaaccaggaccagggtgcggcagcgctgggctgtccacctctgccttttagtttttacttcttctttctttggaggcagaaattgggcataagacaatatgaggggtggtctcctcccttaatcccagcactttgggaggccaaggaaggcggattacgaagtcaggagtttgagactagcctgaccaaaatggtgaaaccccgtctctgctaaaaatacaaaaattagtcggacatagtggcgtgtgcctgtaaccccagctactcaggaggctgaggcaggagaatcgcttgaacctgggaggcagaggttgcagtgagctgagatggagccactgcactgcagcttgggtgacagagcaagaccctgtctcaaaaaaaaaagttatacacatgtaattattgcatgttccttcattattagttttaacatagacttgttaatctcaattagcttaattagcatttgagtttattgctaaaacttctttatgagttttaataatgaggcttggccgagtgtggtgggtcatacctgtaatcccagcatttgggaggccaaggcgggtagatcacttgaggtcaggagttcaagaccagcctggccaacatggttaaaccccatctctactaaaaatacaaaaaaattagctgggcatggtggcacatgcctataatcccagctgctcaggaggctgaggtggaagaatcgcttgaacccagggggcagaggttgcagtgagccgagatcgtgccattgcactctagcctgggcaacagagcaagatcgtctaacaacaacaaaaaaaaaaccaaggctgaatttcttgagtgattgagcagtggctatctattggacagtccagctgaacagtattttcctcaggctgggtgcagtaactcacacctataattccggcacattgggtggctgaggtgggcagatcacttgaggccaggagtgagaccagcctgggtaacgtgcctagactatatctctacaaaaaattttttaaattaagtggcccagtggccagctgtagtcccaactccttgggaggctgaggcaggggggtcacttgagctaggagcttaaggctgtggggagccatgattgcatcaccacgctccagcctgggtgacagagtgacctcgtctcaaggctgcagggagccatgattgcatcactgcactccagcctgggtgacagagagaggcctcgtctcaaggctgcggggagctgtgattgcatcactgcactctagcctgggtgacagagtgacctcgtctcaaggctagagggagccatgattgcatcactgcactccagcctgggtgacagagagagacctcgtctcaaggctgcagggagtcatgattgcatcgctgcactctagcctgggtgacagagaccttgtctcaaggctgcggggagtcatgattgcattgctgcactctagcctgggtgacagagagaccttgtctcaaggctgtggggagtcgtgattgcatcactgcactctagcctgggtgacagagagacctcgtctcaaggtatgtttcatgtatcttctcttttttcattgataaaggcccaaacttcccagagagaaaaacatacagccttaaggaatttggctagaagtctattcagggcatatgatacgatagaacagggattctgtagaacctggaagaaagtagtcactctaggagtaggttagcttgagagaagtagcaagaatgtacttaaagcagcagataatgagatagaattggggtaaattgcggtaggaatatgttagaagcaagggtggaactgtcgtcactgttacctcgatggcgaagccagaatgtgaggctcttgctcttagaactcacgtgagtaccatagcctcgcgttgtcttgcaggacacagattatccttatttgggtaccaccatggacacctataaggaactgattgagaggattcctgagttgaacaaagtggcacgagcagctgcagaggtcgctggtcagttcgtgattaaactaacccatgatgttgaattgaacctggactatgagaggtacaacagccaactgctttcatttgtgagggatctgaaccaatacagagcagacataaaggtgagcactgattccaattacgtttttattttgctgaatgtcaagtattttgaaatgtgatgtgttcctgtgtgttcctgttggaagggtgattgtagccatagtacattttaaagtgaactgaggtataattgtatgtagaattggtaacttgtttgagagaagtcgggaggctgtggattagagacctaggacagagctcagcaggtgtttcagaatccagagcagtgtcaggttttctgtcactcatgtctcccaggcagcccgtcagtaggacacggaatatgaagatctcagcaaggagttgggctgtgtgcctctcgggcgtgacccggatggaaagacagcacagctagcaggattccatctcgtagtgatctgcgcatctaaaagtcaaatattctattaaacgaaactgataagcagggtgaggtggtgcatacccgtagccccagctacttctgctgaggcaggaggattgctggagtccagcccaggcaacatagcaaaatcccatctctaaataaattaataaaactaatattaaagtagcttccagattgttttatggtactaggagttgatttttaacagatctcttaattgaagtaaatcactgacaaccgaatctttttatatcttttttatttttatttttattttattttttttgagagatgaagtctcgctcttgtcccccaggctggaatgcaatgacatgatctcggctcactgcaacctccacctcccggctttaagcgattctcctgcctcggctccccaggtagctgggattacaggcgtgtgccaccatgcccagatagtttttgtgtttttagtagaagccggggttttaccatgttggccaggctgaagtgcagtggcgagatctcggctcactgtaagatgcgcctcccgggttcacgccattctcctgcctaagcctcccgagtagctgggactacaggtgccggccaccacgcccggctaattttttgtatttttagtagagccggggttttaccatgttggccaggctggtcttgaactcctgacctcaggtgatccacccacctcggcctcccaaagtgctgggatcacaggcatgagccaccacgcccggcatcttatgtctttcttgaaactaattgtaactgttgaaaatggaacttaccaggtggaaataactaaatcctgaagtaccttgaaccaaaatgttttcccctatagggacattttcccccaaaaggagaattgaactggaaacaatatagtacataggattatataattatgttcaatttcttaatgagaatggttttcttacatgctgggctcaaatatgagtgtatatcacagtccatagagcttggaacccctgtgcaaggtgctttcggagtcttgagcttatctgcgagttccttttatcagaatcttacttaacgcacgttaaattagaaaggcatacaaaagaatgtccttagaaataaaacttctcatagcgaataatgtctgtttcaggaaatgggcctgagtttacagtggctgtattctgctcgtggagacttcttccgtgctacttccagactaacaacagatttcgggaatgctgagaaaacagacagatttgtcatgaagaaactcaatgatcgtgtcatgagagtaagtgaacttttgggaaaggaggaactaaagtatgtgtaaaataaccgataaatcttacacttctgcaaagtggacaaactctaggagtctagaattcctttaagaagggagcattaatggtttagctgtcattttctgtttctgctgtctaattcagaacttagtcaaacctagtctttttggaagagacttgctgtaaaacttccatgtatgctccaatggggaaaagaatctgaacacatttaaagttttcctttgtaaaatgaatcagtttcctttaaaaaaattttttttttttttgagacagagtttcactgttgttgcccaagctggagtgcaatggcacggtctcggctcactgcaccctccacctcccaggttcaagtgattctcctgccttagcctcccgagttgctgtgattacaggtgcccaacaccacgcccggctcattttttgtatttttagtagaaacggagtttcaccatgttagccaggccggtctcgaactcccaacctcaagtgatccacctgccttggcctcccaaagtgctggtattacaagcgtgagccgctgtgcctagcctcctttagaattttaaccttagaagattagcattagcctggttctcagcattcttttttccttactctgctatagaaagtctgatcagctggctgggtacagtggctcatgcctgtaattccagtactttgggaggccgaggcaagcggatcacctgaggtcaggagttcaagaccagcctgaccaacatggagaaaccccatctctactaaaaatacaaaaattagctgggcgtggtggtgcatgcctgtaattccagctgctcaggaggctgaggcaggagaattacttgaacccgggaggtggaggttgcagtgagctgagatcgcgccattgtactccagcctgggcaacaagagcgaaactctgtctcaacaacaacaaaaagccgggcacggtggctcacacctgtaatcccagcatgaattgcttgaactcgggaggtggagggtaccagtgagccgagatagcgctgttgcactccagtctgggcaacaagagcgaaactctgtgtcaaaaaaaaaaaaaaaaaaaaaaagtctgatcggcattcttaaatttgggacattttacatttgaagtgaactgttgttttactacaaaagtcacagggctgtgtaaattgccttgtgtgttgttttcgtaggtggagtatcacttcctctctccctacgtatctccaaaagagtctcctttccgacatgtcttctggggctccggctctcacacgctgccagctttactggagaacttgaaactgcgtaaacaaaataacggtgcttttaatgaaacgctgttcagaaaccagttggctctagctacttggactattcagggagctgcaaatgccctctctggtgacgtttgggacattgacaatgagttttaaatgtgatacccatagcttccatgagaacagcagggtagtctggtttctagacttgtgctgatcgtgctaaattttcagtagggctacaaaacctgatgttaaaattccatcccatcatcttggtactactagatgtctttaggcagcagcttttaatacagggtagataacctgtacttcaagttaaagtgaataaccacttaaaaaatgtccatgatggaatattcccctatctctagaattttaagtgctttgtaatgggaactgcctctttcctgttgttgttaatgaaaatgtcagaaaccagttatgtgaatgatctctctgaatcctaagggctggtctctgctgaaggttgtaagtggtcgcttactttgagtgatcctccaacttcatttgatgctaaataggagataccaggttgaaagaccttctccaaatgagatctaagcctttccataaggaatgtagctggtttcctcattcctgaaagaaacagttaactttcagaagagatgggcttgttttcttgccaatgaggtctgaaatggaggtccttctgctggataaaatgaggttcaactgttgattgcaggaataaggccttaatatgttaacctcagtgtcatttatgaaaagaggggaccagaagccaaagacttagtatattttcttttcctctgtcccttcccccataagcctccatttagttctttgttatttttgtttcttccaaagcacattgaaagagaaccagtttcaggtgtttagttgcagactcagtttgtcagactttaaagaataatatgctgccaaattttggccaaagtgttaatcttaggggagagctttctgtccttttggcactgagatatttattgtttatttatcagtgacagagttcactataaatggtgtttttttaatagaatataattatcggaagcagtgccttccataattatgacagttatactgtcggttttttttaaataaaagcagcatctgctaataaaacccaacagatactggaagttttgcatttatggtcaacacttaagggttttagaaaacagccgtcagccaaatgtaattgaataaagttgaagctaagatttagagatgaattaaatttaattaggggttgctaagaagcgagcactgaccagataagaatgctggttttcctaaatgcagtgaattgtgaccaagttataaatcaatgtcacttaaaggctgtggtagtactcctgcaaaattttatagctcagtttatccaaggtgtaactctaattcccattttgcaaaatttccagtacctttgtcacaatcctaacacattatcgggagcagtgtcttccataatgtataaagaacaaggtagtttttacctaccacagtgtctgtatcggagacagtgatctccatatgttacactaagggtgtaagtaattatcgggaacagtgtttcccataattttcttcatgcaatgacatcttcaaagcttgaagatcgttagtatctaacatgtatcccaactcctataattccctatcttttagttttagttgcagaaacattttgtggtcattaagcattgggtgggtaaattcaaccactgtaaaatgaaattactacaaaatttgaaatttagcttgggtttttgttacctttatggtttctccaggtcctctacttaatgagatagtagcatacatttataatgtttgctattgacaagtcattttaactttatcacattatttgcatgttacctcctataaacttagtgcggacaagttttaatccagaattgaccttttgacttaaagcagagggactttgtatagaaggtttgggggctgtggggaaggagagtcccctgaaggtctgacacgtctgcctacccattcgtggtgatcaattaaatgtaggtatgaataagttcgaagctccgtgagtgaaccatcattataaacgtgatgatcagctgtttgtcatagggcagttggaaacggcctcctagggaaaagttcatagggtctcttcaggttcttagtgtcacttacctagatttacagcctcacttgaatgtgtcactactcacagtctctttaatcttcagttttatctttaatctcctcttttatcttggactgacatttagcgtagctaagtgaaaaggtcatagctgagattcctggttcgggtgttacgcacacgtacttaaatgaaagcatgtggcatgttcatcgtataacacaatatgaatacagggcatgcattttgcagcagtgagtctcttcagaaaacccttttctacagttagggttgagttacttcctatcaagccagtacgtgctaacaggctcaatattcctgaatgaaatatcagactagtgacaagctcctggtcttgagatgtcttctcgttaaggagatgggccttttggaggtaaaggataaaatgaatgagttctgtcatgattcactattctagaacttgcatgacctttactgtgttagctctttgaatgttcttgaaattttagactttctttgtaaacaaatgatatgtccttatcattgtataaaagctgttatgtgcaacagtgtggagattccgtcgagataacttcgtataatgtatgctatacgaagttatgctagtaactataacggtcctaaggtagcgagctagcgctggtttgcatttccctaaattagacagtaactttcagaagagatggaacttgttttcttgccagcaaggttgaaaataggtccttagctggaccaatttgtctgtttacaggaataagtaaggccttactggttaaccttggtgttctttatgatgagaccagaagccaaagacctcaagttttctctcctcttggcatctgccctatagtctttagttctttgtttttattttgtttttctttttttcccaacattttctgaaaaagaacaagttttagactcaatttgtcagacttgaaaagaacacactgccaagttttggccaaagtgttagtcttcaggaaagctttctatcattttggcactgagatatttattgtttatttatcagtgacagagttcactataaatagtgttgttttttttttatatatagaagataattatcggaagcagtgccttccataattatgacagttatactgtcgttttcttttaataaaagcagcatctgctaatgagacccacagatactggaagttttgcacttatggtcagcacttgcaactttaggaaggaagaatgccacaatccaaataatatgagctagaagaggattgggttaaataagagattcctaattgagttggggaaaaaaatgatagttttcctaagtgcagtgagttgtggccaagttaaatgtcatttaaaggctatggtagtacttacatctacaaaattttacagctcaatttattcaagatgtaactcaaaatcaattttgcaaaatttccagtacctttgtcacaaacttaactcacattatcgggagcagtgtcttccataatgcataaagaacaaggtagtttttgcctaccacag

SEQ ID NO: 11 - DNA, 24 bp, synthetic construct
  source 1..24: mol_type = other DNA
cccaagaggtttaatggaagactc

SEQ ID NO: 12 - DNA, 24 bp, synthetic construct
  source 1..24: mol_type = other DNA
ggccactcattcttaccaatcaag

SEQ ID NO: 13 - DNA, 25 bp, synthetic construct
  source 1..25: mol_type = other DNA
tgctttgcagctattgcactagtca

SEQ ID NO: 14 - DNA, 22 bp, synthetic construct
  source 1..22: mol_type = other DNA
ctgactgagaagaccagtgtga

SEQ ID NO: 15 - DNA, 23 bp, synthetic construct
  source 1..23: mol_type = other DNA
agggtgttctcctttcaaaccaa

SEQ ID NO: 16 - DNA, 20 bp, synthetic construct
  source 1..20: mol_type = other DNA
ctagcccttgactgggccca

SEQ ID NO: 17 - DNA, 21 bp, synthetic construct
  source 1..21: mol_type = other DNA
ggagggtcaacgtggtagttg

SEQ ID NO: 18 - DNA, 21 bp, synthetic construct
  source 1..21: mol_type = other DNA
aagggcatacagctcaatgga

SEQ ID NO: 19 - DNA, 23 bp, synthetic construct
  source 1..23: mol_type = other DNA
tagttggagattgccccgtctcc

SEQ ID NO: 20 - DNA, 21 bp, synthetic construct
  source 1..21: mol_type = other DNA
gcatgtggcatgttcatcgta

SEQ ID NO: 21 - DNA, 21 bp, synthetic construct
  source 1..21: mol_type = other DNA
agcacgtactggcttgatagg

SEQ ID NO: 22 - DNA, 27 bp, synthetic construct
  source 1..27: mol_type = other DNA
atgaatacagggcatgcattttgcagc

SEQ ID NO: 23 - DNA, 20 bp, synthetic construct
  source 1..20: mol_type = other DNA
gctgcattgcggactgtaga

SEQ ID NO: 24 - DNA, 23 bp, synthetic construct
  source 1..23: mol_type = other DNA
tccatcattctcagctgctacaa

SEQ ID NO: 25 - protein, 760 aa, synthetic construct; identical to SEQ ID NO: 4

SEQ ID NO: 26 - protein, 67 aa, Homo sapiens
MMDQARSAFSNLFGGEPLSYTRFSLARQVDGDNSHVEMKLAVDEEENADNNTKANVTKPKRCSGSIC

SEQ ID NO: 27 - protein, 21 aa, Homo sapiens
YGTIAVIVFFLIGFMIGYLGY

SEQ ID NO: 28 - protein, 672 aa, Homo sapiens
CKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 29 - DNA, 20 bp, synthetic construct
  source 1..20: mol_type = other DNA
ggcagcatgaatacttctga

SEQ ID NO: 30 - DNA, 20 bp, synthetic construct
  source 1..20: mol_type = other DNA
accacggagttcgaacagag

SEQ ID NO: 31 - DNA, 20 bp, synthetic construct
  source 1..20: mol_type = other DNA
aatataccctacctgtggca

SEQ ID NO: 32 - DNA, 20 bp, synthetic construct
  source 1..20: mol_type = other DNA
ttggtggaggagacacgcca

SEQ ID NO: 33 - DNA, 23 bp, synthetic construct
  source 1..23: mol_type = other DNA
ctcatgcttcgggagttaatgct

SEQ ID NO: 34 - DNA, 23 bp, synthetic construct
  source 1..23: mol_type = other DNA
ctcatgcttcgggagttaatgct

SEQ ID NO: 35 - DNA, 21 bp, synthetic construct
  source 1..21: mol_type = other DNA
ggtcggtacgtcttccacagt

SEQ ID NO: 36 - DNA, 19 bp, synthetic construct
  source 1..19: mol_type = other DNA
gtgtgtctggtgaccctga

SEQ ID NO: 37 - DNA, 22 bp, synthetic construct
  source 1..22: mol_type = other DNA
aaccgtcctctgtgctgagccc

SEQ ID NO: 38 - DNA, 19 bp, synthetic construct
  source 1..19: mol_type = other DNA
gtgggccttctggaaatgg

SEQ ID NO: 39 - DNA, 22 bp, synthetic construct
  source 1..22: mol_type = other DNA
gggaccaacctagctcacatct

SEQ ID NO: 40 - DNA, 23 bp, synthetic construct
  source 1..23: mol_type = other DNA
tgcctcttccaggacatgaacga

SEQ ID NO: 41 - DNA, 20 bp, synthetic construct
  source 1..20: mol_type = other DNA
ccctgctgagagcctctaac

SEQ ID NO: 42 - DNA, 18 bp, synthetic construct
  source 1..18: mol_type = other DNA
gcgatggagccgtggatt

SEQ ID NO: 43 - DNA, 22 bp, synthetic construct
  source 1..22: mol_type = other DNA
acctcaggctgttacagggctg

SEQ ID NO: 44 - DNA, 20 bp, synthetic construct
  source 1..20: mol_type = other DNA
ccctcccagttcaggtctca

SEQ ID NO: 45 - DNA, 18 bp, synthetic construct
  source 1..18: mol_type = other DNA
gcctggccatccctgtct

SEQ ID NO: 46 - DNA, 25 bp, synthetic construct
  source 1..25: mol_type = other DNA
cactgctgatgggagagctgtttca

SEQ ID NO: 47 - DNA, 22 bp, synthetic construct
  source 1..22: mol_type = other DNA
gcctctgtaaacgacggactct

SEQ ID NO: 48 - protein, 953 aa, Mus musculus
MNIRKPLCSNSVVGACTLISLTTAVILGHLMLRELMLLPQDLHESSSGLWKTYRPHHQEGYKPGPLHIQEQTEQPKEAPTQCDVPPSSRFDCAPDKGISQEQCEARGCCYVPAGQVLKEPQIGQPWCFFPPSYPSYRLENLSSTESGYTATLTRTSPTFFPKDVLTLQLEVLMETDSRLHFKIKDPASKRYEVPLETPRVLSQAPSPLYSVEFSEEPFGVIVRRKLGGRVLLNTTVAPLFFADQFLQLSTSLPSQHITGLGEHLSPLMLSTDWARITLWNRDTPPSQGTNLYGSHPFYLALEDGGLAHGVFLLNSNAMDVILQPSPALTWRSTGGILDVYVFLGPEPKSVVQQYLDVVGYPFMPPYWGLGFHLCRWGYSSTAIVRQVVENMTRTHFPLDVQWNDLDYMDARRDFTFNQDSFADFPDMVRELHQDGRRYMMIVDPAISSAGPAGSYRPYDEGLRRGVFITNETGQPLIGKVWPGTTAFPDFTNPETLDWWQDMVSEFHAQVPFDGMWLDMNEPSNFVRGSQQGCPNNELENPPYVPGVVGGILQAATICASSHQFLSTHYNLHNLYGLTEAIASSRALVKTRGTRPFVISRSTFSGHGRYAGHWTGDVRSSWEHLAYSVPDILQFNLLGVPLVGADICGFIGDTSEELCVRWTQLGAFYPFMRNHNDLNSVPQEPYRFSETAQQAMRKAFALRYALLPYLYTLFHRAHVRGDTVARPLFLEFPEDPSTWSVDRQLLWGPALLITPVLEPGKTEVTGYFPKGTWYNMQMVSVDSLGTLPSPSSASSFRSAVQSKGQWLTLEAPLDTINVHLREGYIIPLQGPSLTTTESRKQPMALAVALTASGEADGELFWDDGESLAVLERGAYTLVTFSAKNNTIVNKLVRVTKEGAELQLREVTVLGVATAPTQVLSNGIPVSNFTYSPDNKSLAIPVSLLMGELFQISWS

SEQ ID NO: 49 - DNA, 2862 bp, Mus musculus
  source 1..2862: mol_type = other DNA
atgaatatacggaagcccctctgttcgaactccgtggttggggcctgcacccttatctctctgactacagcggtcatcctgggtcatctcatgcttcgggagttaatgctgcttccccaagaccttcatgagtcctcttcaggactgtggaagacgtaccgacctcaccaccaggaaggttacaagccagggcctctgcacatccaggagcagactgaacagcccaaagaagcacccacacagtgtgatgtgccccccagcagccgctttgactgtgcccccgacaaaggcatctcacaggagcaatgcgaggcccgcggctgctgctatgtcccagcagggcaggtgctgaaggagccgcagatagggcagccctggtgtttcttccctcccagctacccaagctaccgtctagagaacctgagctctacagagtcggggtacacagccaccctgacccgtaccagcccgaccttcttcccaaaggatgtgctgaccttacagctggaggtgctgatggagacagacagccgcctccacttcaagatcaaagatcctgctagtaagcgctacgaagtgcccctggagaccccacgtgtgctgagccaggcaccatccccactttacagcgtggaattctcagaggaaccctttggagtgatcgttcgtaggaagcttggtggccgagtgttgctgaacacaaccgtggcccccctgttcttcgctgaccagttcctgcagctgtccacttccctgccctcccagcacatcacaggcctgggggaacacctcagcccactcatgctcagcaccgactgggctcgtatcaccctctggaaccgggacacaccaccctcgcaaggtaccaacctctacgggtcacatcctttctacctggcactggaggacggtggcttggctcacggtgtcttcttgctaaacagcaatgccatggatgtcatcctgcaacccagcccagccctaacctggaggtcaacgggcgggatcctggatgtgtatgtgttcctaggcccagagcccaagagcgttgtgcaacaatacctggatgttgtgggataccccttcatgcctccatactggggcctcggcttccacctctgccgctggggctactcctcgaccgccattgtccgccaggtagtggagaacatgaccaggacacacttcccgctggacgtgcaatggaatgacctggactacatggacgcccgaagagacttcaccttcaaccaggacagctttgccgacttcccagacatggtgcgggagctgcaccaggatggccggcgctacatgatgatcgtggaccctgccatcagcagcgcaggccctgctgggagttacaggccctacgacgagggtctgcggaggggcgtgttcatcaccaacgagactgggcagccgctgattgggaaggtttggcctggaaccaccgccttccctgatttcaccaaccctgagacccttgactggtggcaggacatggtgtctgagttccacgcccaggtgcccttcgatggcatgtggctcgacatgaacgaaccgtccaacttcgttagaggctctcagcagggctgccccaacaatgaactggagaaccccccctatgtgcccggggtggttggcgggatcttgcaggcagccaccatctgtgcctccagccaccaattcctctccacacactacaacctccacaacctgtacggcctcactgaagctatcgcctccagcagggccctggtcaagactcggggaacacgaccctttgtgatctcccgctcaaccttctcgggccacggccggtacgctggtcactggacaggggatgtgcggagctcttgggagcatcttgcatactctgtgccagacatcctgcagttcaacctgctgggcgtgcccctggtcggggcggacatctgcggcttcataggagacacgtcagaagagctgtgtgtgcgctggacccagttgggggccttctaccccttcatgcggaaccacaatgacctgaatagcgtgcctcaggagccgtacaggttcagcgagacggcgcagcaggccatgaggaaggccttcgccttacgctatgcccttctgccctacctgtacactctcttccaccgcgcccacgtcagaggagacacggtggcccggcccctcttcctggagttccctgaggatcccagcacctggtctgtggaccgccagctcttgtgggggccggccctgctcatcacacctgtgcttgagcctgggaaaactgaagtgacgggctacttccccaagggcacgtggtacaacatgcagatggtgtcagtggattccctcggtactctcccttctccatcatcggcttcatccttcagatctgctgtccagagcaaggggcagtggctgacactggaagccccactggataccatcaacgtgcacctgagggaggggtacatcataccgctgcagggtcccagcctcacaaccacggagtcccgaaagcagcccatggctctggctgtggcattaacagcaagcggcgaggccgatggggagctgttctgggacgacggggagagcctcgcggttctggagcgtggggcctacacactggtcaccttctcagccaagaacaataccattgtgaacaagttagtgcgtgtgaccaaggagggagctgagctacaactgagggaggtgaccgtcttgggagtggccacagctcctacccaggtcctttccaacggcatccctgtctccaatttcacctacagccctgacaacaagagcctggccatccctgtctcactgctgatgggagagctgtttcaaatcagctggtcctag

SEQ ID NO: 50 - DNA, 425 bp, synthetic construct
  source 1..425: mol_type = other DNA
ctcttccaccttcccactcacccttttctcttacacagaaggccctgcgataaagtcctggctccctcacatctgtctgtacctgtgttgtgggcccatgtctgcagagacaccctctgcctctgaggagtctcccattgtctcctgtacctggcttctgtcttcccttccgttgctctggggaagatgctctggctgcctccttccacctcctggcagtgtgtgtgggtgttgtgtgagaaaccccacccacactgatactggctcctcctctcctcaggccagggcatcaagaacaaccctaggatgggacttctggaatgggccccgagttaggcccaggcagaaatctgtcctggcaagcccagagaaaagttgtatctgtcaaataccaaatgtgtcaccagagttgatgggctgagaga

SEQ ID NO: 51 - DNA, 473 bp, synthetic construct
  source 1..473: mol_type = other DNA
ctcttccaccttcccactcacccttttctcttacacagaaggccctgcgataaagtcctggctccctcacatctgtctgtacctgtgttgtgggcccatgtctgcagagacaccctctgcctctgaggagtctcccattgtctcctgtacctggcttctgtcttcccttccgttgctctggggaagatgctctggctgcctccttccacctcctggcagtgtgtgtgggtgttgtgtgagaaaccccacccacactgatactggctcctcctctcctcatgctgccccgaaccaacaggctttcaccatgaatatacggaagcccctctccagggcatcaagaacaaccctaggatgggacttctggaatgggccccgagttaggcccaggcagaaatctgtcctggcaagcccagagaaaagttgtatctgtcaaataccaaatgtgtcaccagagttgatgggctgagaga

SEQ ID NO: 52 - protein, 883 aa, synthetic construct
AHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC